(19) United States
(12) Patent Application Publication
(10) Pub. No.: US 2023/0391714 A1
(43) Pub. Date: Dec. 7, 2023

(54) INHIBITORS OF THE INTERACTION BETWEEN TRIP8B AND HCN CHANNELS AND USES THEREOF FOR TREATING NEUROLOGICAL DISEASES AND DISORDERS

(71) Applicant: Northwestern University, Evanston, IL (US)

(72) Inventors: Gary E. Schiltz, Naperville, IL (US); Iredia David Iyamu, Evanston, IL (US); Ye Han, Evanston, IL (US); Dane M. Chetkovich, Evanston, IL (US); Kyle A. Lyman, Evanston, IL (US)

(21) Appl. No.: 18/330,874

(22) Filed: Jun. 7, 2023

Related U.S. Application Data
(60) Provisional application No. 63/349,886, filed on Jun. 7, 2022.

(51) Int. Cl. C07C 235/34; C07D 309/04; C07D 239/52; C07D 211/26; C07D 213/58; C07D 333/28; C07D 231/12; C07D 209/30; C07D 215/04; C07D 305/08; C07D 401/12

(52) U.S. Cl. CPC C07C 235/34; C07D 309/04; C07D 239/52; C07D 211/26; C07D 213/58; C07D 333/28; C07D 231/12; C07D 209/30; C07D 215/04; C07D 305/08; C07D 401/12

(57) ABSTRACT

Disclosed herein are substituted phenyl compounds of formula I as defined herein that may be utilized as inhibitors of the interaction between the subunits of hyperpolarization-activated cyclic-nucleotide gated (HCN) channels, such as HCN1, and an auxiliary subunit of HCN channels which is the tetratricopeptide repeat-containing Rab8b-interacting protein (TRIP8b). The disclosed compounds may be used in pharmaceutical compositions and methods for treating neurological diseases and disorders such as depression, and in particular Major Depressive Disorder (MDD).

### CROSS-REFERENCE TO RELATED APPLICATIONS

This application claims priority to U.S. Provisional Application No. 63/349,886 filed on Jun. 7, 2022, the contents of which are incorporated by reference in their entireties.

### STATEMENT REGARDING FEDERALLY SPONSORED RESEARCH OR DEVELOPMENT

This invention was made with government support under grant numbers CA060553, MH106511, and MH128747 awarded by the National Institutes of Health. The government has certain rights in the invention.

### BACKGROUND

The field of the invention relates to compounds which act as inhibitors of the interaction between the subunits of hyperpolarization-activated cyclic-nucleotide gated (HCN) channels and an auxiliary subunit of HCN channels which is the tetratricopeptide repeat-containing Rab8b-interacting protein (TRIP8b). The disclosed compounds may be utilized in methods for treating neurological diseases and disorders such as Major Depressive Disorder (MDD).

Major Depressive Disorder (MDD) is a critical public health problem with a lifetime prevalence of nearly 17% in the United States. Many patients remain symptomatic despite treatment for MDD, which highlights a need to develop mechanistically distinct antidepressant therapies. One target that has gained attention is the interaction between the subunits of hyperpolarization-activated cyclic-nucleotide gated (HCN) channels and an auxiliary subunit of HCN channels which is the tetratricopeptide repeat-containing Rab8b-interacting protein (TRIP8b).

HCN channels belong to the superfamily of voltage-gated K+ (Kv) and cyclic nucleotide-gated (CNG) channels and consist of four either identical or non-identical subunits that are integrally embedded in the cell membrane to create an ion-conducting pore. HCN channels are encoded by four genes (HCN1, 2, 4, and 4) which are expressed in the central nervous system (CNS) and heart. The subunits of HCN channels may bind auxiliary subunits, one of which is tetratricopeptide repeat-containing Rab8b-interacting protein (TRIP8b). TRIP8b is known to bind to the subunits of HCN channels, such as HCN1, and regulate HCN channel activity.

HCN channels that are expressed in the CNS are known to modulate neurons' response to synaptic input. In particular, HCN channels are key regulators of neuronal excitability in the mammalian hippocampus, and recent work by the inventors and others has established that antagonizing either HCN or TRIP8 promotes antidepressant-like effects on behavior. Accordingly, the inventors previously developed a high-throughput screening assay to identify small-molecules that are capable of disrupting the TRIP8b-HCN interaction. (See Han et al., “Method for Identifying Small Molecule Inhibitor of the Protein-Protein Interaction Between HCN1 and TRIP8b,” J. Vis. Exp. Nov 11; (117):54540, the content of which is incorporated herein by reference in its entirety).

Here, the inventors disclose the results of a virtual high-throughput virtual screen which was used to identify chemical scaffolds that are capable of disrupting the TRIP8b-HCN interaction. In particular, the inventors' screen identified compound NUCC-0200590 as a compound that is capable of disrupting the TRIP8b-HCN interaction. The activity of compound NUCC-0200590 subsequently was characterized using biochemical and biophysical assays. The inventors found that compound NUCC-0200590 is capable of disrupting the TRIP8b-HCN interaction in a series of cell-based assays as well as in vitro experiments in the mouse hippocampus. The inventors results provide a template for the development of small molecules that are capable of disrupting the TRIP8b-HCN interaction and could have utility as a new class of drugs for treating neurological diseases and disorders such as depression and in particular Major Depressive Disorder (MDD).

### SUMMARY

Disclosed herein are compounds which may be utilized as inhibitors of the interaction between the subunits of hyperpolarization-activated cyclic-nucleotide gated (HCN) channels, such as HCN1, and an auxiliary subunit of HCN channels which is the tetratricopeptide repeat-containing Rab8b-interacting protein (TRIP8b). The disclosed compounds may be used in pharmaceutical compositions and methods for treating neurological diseases and disorders such as depression, and in particular Major Depressive Disorder (MDD).

The disclosed compounds may include compounds having a formula I:

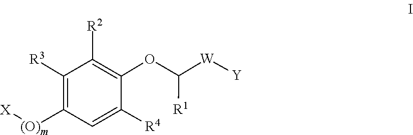

wherein:
X is phenyl optionally substituted at one or more positions with halogen, alkyl, alkoxy, hydroxyl, carboxamido, hydroxyl, cyano, nitro, haloalkyl, alkylthio, alkenyl, amino, alkylsulfonyl, hydroxyalkyl, or phenyl optionally substituted at one or more positions with halogen;
or X is pyrimidinyl optionally substituted at one or more positions with alkoxy;
or X is pyridinyl optionally substituted at one or more positions with halogen or haloalkyl;
or X is pyrazolyl optionally substituted at one or more positions with positions with alkyl or haloalkyl;
or X is thiophenyl optionally substituted at one or more positions with halogen
or X is cycloalkyl;
or X is halogen or haloalkyl;
m is 0 or 1;
R¹ is H or alkyl (e.g., methyl or ethyl);
W is —CH₂— or —C(O)—;
Y is —NH-(Alk)ₙ-Z or —N(R⁵)—(Alk)ₙ-Z, wherein Alk is —CH₂— or —CH(CH₃)—, R⁵ is methyl or together with R⁴ forms a hetrocycle, and n is 0-2;
or Y is

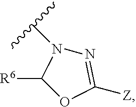

wherein R⁶ together with R⁴ forms a hetrocycle;
or Y is —NH—S(O)₂—Z;
or Y is carboxyl, amino, alkylamino, dialkylamino, indolinyl optionally substituted at one or more positions with halogen; indanyl, 1,2,3,4-tetrahydroisoquinolinyl, or isoindolinyl;
Z is phenyl optionally substituted at one or more positions with halogen, alkyl, haloalkyl, aminosulfonyl, alkoxy, pyrazolyl, imidazolyl, alkylsufonyl, alkylaminocarbonyl, hydroxyl, cyano, nitro, alkenyl, aminoalkyl, or hydroxyalkyl;
or Z is 1,3-benzodioxole; piperidinyl; pyridinyl optionally substituted with alkoxy, benzothiazole; indolinyl optionally substituted with halogen or alkyl; cycloalkyl, or a cyclic ether;
R² is —CH₂—Het, wherein Het is a saturated heterocycle comprising 5, 6, or 7 atoms wherein at least one of the atoms is a nitrogen atom (e.g., piperazinyl, 1,4-diazepanyl, morpholinyl, piperidinyl, pyrrolidinyl), and the heterocycle is optionally substituted at one or more positions with alkyl, amino, alkylamino, dialkylamino, alkoxycarbonyl, or alkylsulfonyl;
or R² is H, —CH₂—NH₂, —C(O)—NH₂, —C(O)—OH, —C(NH₂)═N—OH, —CH₂—NH—CH₂—CH₂—N═C(NH₂)₂, —CH₂—NH—CH₂—CH₂—NH₂, —CH₂—NH—CH₂—CH₂—N(CH₃)₂, —CH₂—NH—CH₂—C(O)—NH₂, —CH₂—NH—CH₂—CH₂—NH—CH₃, —CH₂—NH—CH₂—CH₂—N═C(NH₂)₂, or —CH₂—NH—CH(NH₂)—N═NH; or R₂ is

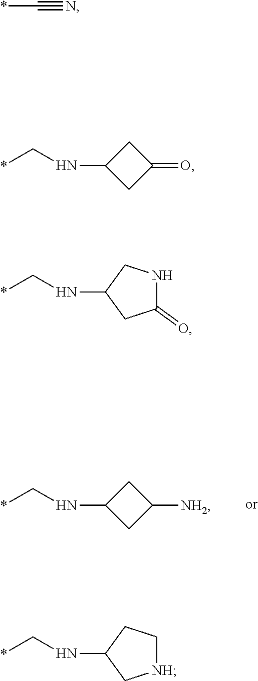

R³ is H or —CH₂—NH—CH₂—CH₂—N═C(NH₂)₂; and
R⁴ is H, —CH₂—NH₂, —C(O)—NH₂, or —C(NH₂)═N—OH.

The disclosed compounds may exhibit one or more biological activities. The disclosed compounds may inhibit an interaction between TRIP8b and one or more subunits of the HCN, such as an interaction between TRIP8b and HCN1. The disclosed compounds may inhibit the interaction between TRIP8b and one or more subunits of the HCN, such as an interaction between TRIP8b and HCN1, in vitro, ex vivo, or in vivo. Determination of the compounds ability to inhibitor the interaction between TRIP8b and the one or more subunits of HCN may be accomplished by the assay methods described herein.

Also disclosed are pharmaceutical compositions comprising the disclosed compounds and a suitable pharmaceutical carrier, excipient, or diluent. The disclosed pharmaceutical compositions may comprise an effective amount of the compound for inhibiting an interaction between TRIP8b and one or more subunits of the HCN, such as an interaction between TRIP8b and HCN1.

Also disclosed are methods for treating neurological diseases and disorders such as depression and particularly Major Depressive Disorder (MDD). The methods may include administering the disclosed compounds or pharmaceutical compositions comprising the disclosed compounds to a subject in need thereof, for example, to a subject having a neurological disease or disorder. The disclosed compounds or pharmaceutical compositions comprising the disclosed compounds may be administered with additional therapeutic agents, optionally in combination, in order to treat neurological diseases and disorders. Neurological diseases and disorders treated by the disclosed methods may include, but are not limited to, mood disorders such as major depression, dysthymia, bipolar disorder, mood disorders related to another health condition, and substance-induced mood disorders.

### BRIEF DESCRIPTION OF THE FIGURES

FIG. 1. (A)¹H-NMR reference spectrum of a 2 mM solution of NUCC-0200590 (HCl salt) in deuterated PBS; (B) STD-NMR of 2 mM solution of NUCC-0200590 (HCl salt) and 20 μM of TRIP8b in deuterated PBS; (C) STD-NMR of 2 mM solution of NUCC-0200590 (HCl salt) in deuterated PBS without TRIP8b.

FIG. 2. Epitope Mapping of inhibitor NUCC-0200590.

FIG. 3. HEK293T cells were incubated with varying concentrations of NUCC-0200590 (0-50 μM) for 24 hours and then the viability assayed with a commercially available assay (see Examples). Data are expressed as a percentage of the viability in the 0 μM NUCC-0200590 control condition and error bars denote SEM. One-way ANOVA showed a main effect of concentration (F(6,20)=138.84, p<0.01). * denotes p<0.05 on Tukey's post-hoc tests for pairwise comparisons of 25 μM condition with 0.8 μM-12.5 μM conditions and pairwise comparisons of 50 μM condition with 0.8 μM-12.5 μM conditions.

FIG. 4. NanoBiT assay confirms that NUCC-0200590-HCl disrupts the interaction between fragments of TRIP8b and HCN1 in live cells. HEK293T cells were transfected and 24 hours later were incubated with NUCC-0200590 for 30 min. As a control (black dots), fragments of control proteins PRKACA and PRKAR2A were incubated with NUCC-0200590 for 30 min.

FIG. 5. NUCC-0200590 disrupts TRIP8b mediated HCN channel regulation in HEK cells. A-D) Representative whole cell recordings were made from HEK cells stably expressing HCN2 and transiently transfected with an empty plasmid (A/B) or TRIP8b (C/D). Cells were also incubated for 24 hours with NUCC-0200590 (B/D). E) The V50 of Ih was measured in the presence of increasing concentrations of NUCC-0200590 after incubation for 24 hours. Note that the Y axis is negative such that more hyperpolarized values are higher up on the plot compared with less hyperpolarized values. At 0 μM NUCC-0200590, TRIP8b transfection hyperpolarized the V50 of Ih as has previously been shown. In the presence of NUCC-0200590, there was no effect of TRIP8b on the V50 of Ih. 2-way ANOVA of V50 with factors transfection (empty plasmid, TRIP8b) and drug concentration (0, 2.5, 5, 7.5, 10 μM) showed no effect of transfection (F(1,147)=1.27, p=0.26) but an effect of drug (F(4,147)=6.78, p=0.0000485) and an interaction between the two conditions (F(4,147)=5.34, p=0.00047). Planned post-hoc comparisons with Bonferroni correction for multiple comparisons (p<0.01 significant) revealed a difference between the two transfection conditions only at 0 μM (t(78)=−4.9361, p=0.000004) but not at other drug concentrations 2.5 μM (t(17)=0.50, p=0.62), 5 μM (t(18)=1.31, p=0.20), 7.5 μM (t(17)=1.10, p=0.28), or 10 μM (t(17)=0.96, p=0.34). For the HCN2 alone condition, n=33, 6, 6, 6, 6 cells for the 0, 2.5, 5, 7.5, 10 μM condition while for the HCN2+TRIP8b condition n=61, 13, 14, 13, 13 cells. F) Transfection with TRIP8b Increased the surface expression of HCN channels and led to a higher density of Ih, consistent with prior reports. However, increasing concentrations of NUCC-0200590 led to less current density of Ih, indicating disruption of the TRIP8b-HCN interaction. 2-way ANOVA of current density with factors transfection (empty plasmid, TRIP8b(1a-4)) and drug concentration (0, 2.5, 5, 7.5, 10 μM) showed an effect of transfection (F(1,148)=90.76, p=4.4712E-17), an effect of drug (F(4,148)=7.74, p=0.00001), and an interaction between the two (F(4,148)=7.06, p=0.00003). Planned post-hoc comparisons with Bonferroni correction (p<0.01 significant) revealed a difference between the two transfection conditions at 0-5 M drug concentrations 0 μM (t(78)=−13.41, p=5.9243E-22), 2.5 μM (t(17)=−4.29, p=0.0004), 5 μM (t(18)=−4.12, p=0.0006), 7.5 μM (t(17)=−2.541, p=0.021), or 10 μM (t(17)=−2.56, p=0.019). For the HCN alone condition n=26, 6, 6, 6, 6 cells were used for the 0, 2.5, 5, 7.5, 10 μM concentrations while for the HCN+TRIP8b condition n=54, 13, 14, 13, 14 cells were used. G) Flow cytometry confirms that NUCC-0200590 disrupts TRIP8b mediated HCN1 surface trafficking. 2-way ANOVA of surface HCN1 expression with factors transfection (empty plasmid, TRIP8b) and drug concentration (0, 3, 6, 9, 12, 18 μM) showed an effect of transfection (F(1,98)=7.73, p=8.1086E-21), an effect of drug (F(5,98)=64.668, p=1.6692E-29), and an interaction between the two (F(5,98)=36.37, p=6.6418E-21). Planned post-hoc comparisons with Bonferroni correction (p<0.0083 significant) revealed a difference between the two transfection conditions at 0-9 μM drug concentrations 0 μM (t(20)=−15.8, p=9.09E-13), 3 μM (t(17)=17.68, p=2.21E-12), 6 μM (t(17)=−4.26, p=5.26E-4), 9 μM (t(20)=−5.93, p=8.3E-6), 12 μM (t(14)=0.99, p=0.33), 18 μM (t(10)=1.30, p=0.22). For the HCN1 conditions, n=12,11,8,11,8,7 for 0,3,6,9,12,18 μM conditions while for the HCN1+TRIP8b condition n=10,8,11,11,8,5. H) Current density of Ih was investigated in HEK293 cells stably expressing HCN2 and transiently transfected with TRIP8b. Cells were recorded after varying amounts of time in 5 μM NUCC-0200590. One way ANOVA revealed an effect of time (F(3,52)=4.46, p=0.007) and post-hoc analysis revealed significant differences only when comparing less than 1 hour with greater than 24 hours in 5 μM NUCC-0200590. I) Using the same cells collected for H), we also analyzed the half activation potential (V50) of Ih. One way ANOVA revealed an effect of time (F(3,52)=13.50, p=1.24e-6) and post-hoc analysis revealed differences between less than 1 hour and 1-4 hours (p=0.013), between less than 1 hour and 8 to 10 hours (p=4.38e-5), and between less than 1 hour and greater than 24 hours (p=2.11e-6). For the <1 hour condition 16 cells were used, 1-4 hour condition was 12 cells, 8-10 hour condition was 14 cells, and over 24 hours was 14 cells.

FIG. 6. NUCC-0200590 limits the current mediated by HCN channels in CA1 pyramidal neurons. A) Whole cell recordings were performed from CA1 pyramidal neurons in hippocampal slices made from Trip8b+/+ mice. A subset of slices were incubated with 15 μM NUCC-0200590 (in red). Representative current clamp traces are shown in response to a is long −200 pA somatic current injection from a membrane potential of −70 mV. Note that traces are scaled so that the maximum deflection is 100% to facilitate comparison of the sag ratio. B) Quantification of sag ratio in cells from Trip8b+/+ mice (0 μM/black: 1.22±0.02, 15 μM/red: 1.14±0.01, 2 tail T test yields t=2.97, p=0.0127, n=7,6). C) Whole cell recordings performed from CA1 pyramidal neurons from Trip8b−/− mice incubated with (green) or without (blue) 15 μM NUCC-0200590. D) No differences were noted on 2 tail t-test comparing the sag ratio recorded from Trip8b−/− cells (0 μM/blue: 1.07±0.007, 15 μM/green: 1.05±0.02, 2 tail T test yields t=1.21, p=0.25, n=6,4). E) Excitability of Trip8b+/+ CA1 pyramidal neurons was examined by measuring the number of action potentials fired in response to a is current injection of varying magnitude (see x axis). No difference between the two conditions was noted using repeated measures ANOVA (F(5,55)=0.78, p=0.56, n=7,6). F) The excitability of Trip8b−/− CA1 pyramidal neurons was examined but no difference between the two conditions was noted using repeated measures ANOVA (F(5,40)=0.39, p=0.85, n=6,4).

FIG. 7. Immunohistochemistry reveals that NUCC-0200590 causes a reduction in TRIP8b mediated HCN channel trafficking. Panels A-D) show Trip8b^{+/+} (A/B) and Trip8b^{-/-} (C/D) mice were injected unilaterally with 10 μM NUCC-0200590 and then sacrificed for immunohistochemistry using an anti-HCN1 antibody 48 hours later. For quantification, shown in panel (E), regions of interest were drawn over the injected side (A/C) and scaled by the intensity of the staining on the uninjected side (B/D)) so that a value of ‘1’ reflects similar staining in the two hemispheres while values lower than 1 indicated less HCN1 expression. We observed a significant reduction in HCN1 staining in the injected hemisphere of Trip8b^{+/+} animals by repeated measures ANOVA (F(11,110)=2.76, p=0.003, n=9 Trip8b⁺⁺, n=3 Trip8b^{-/-}). SO: Stratum oriens, SP: Stratum pyramidale, SR: Stratum radiatum, SLM: Stratum lacunosum moleculare.

FIG. 8. Schematic highlighting proposed effect of NUCC-0200590 on TRIP8b mediated HCN channel trafficking. A) Model showing baseline levels of TRIP8b (green) bound to HCN (blue) with relative hyperpolarization of the V50 because of TRIP8b binding. B) Summary of results from our prior report showing that after chronic stress, there is an increase in TRIP8b mediated HCN channel surface expression in the dendrites of CA1 pyramidal neurons (17). The increase in surface expression of HCN channels causes the CA1 pyramidal neurons to be less excitable and ultimately influences cognitive impairment. C-E) Interpretation of our results from HEK cell electrophysiology. C) Overexpression of TRIP8b leads to high levels of surface HCN channel expression with hyperpolarized V50. D) After a brief incubation with NUCC-0200590 (purple), TRIP8b dissociates from HCN channels such that the V50 of HCN depolarizes. Given the short incubation period, the HCN channels remain at the surface of the cell (high current density) but are unbound by TRIP8b. This period is represented in our data by the 1-4 hour incubations in FIG. 4H/4I. E) After 8 or more hours, the HCN channels at the cell surface that are no longer bound by TRIP8b are internalized and there is a reduction in Ih current density. This time point is represented in our data by the >24 hours incubations in FIG. 4H/4I.

### DETAILED DESCRIPTION

The present invention is described herein using several definitions, as set forth below and throughout the application.

Unless otherwise specified or indicated by context, the terms “a” “an”, and “the” mean “one or more.” For example, “a compound” should be interpreted to mean “one or more compounds.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, a “subject” may be interchangeable with “patient” or “individual” and means an animal, which may be a human or non-human animal, in need of treatment.

A “subject in need of treatment” may include a subject having a disease, disorder, or condition that is responsive to therapy with an inhibitor of the binding between TRIP8b and one or more subunits of the HCN channel, such as the presently disclosed compounds. For example, a “subject in need of treatment” may include a subject having a neurological disease, disorder, or condition such as mood disorders (e.g., major depression, dysthymia, bipolar disorder, mood disorders related to another health condition, and substance-induced mood disorders). A “subject in need of treatment” may include a subject having a neurological disease, disorder, or condition such as Major Depressive Disorder that is associated with the binding of TRIP8b to one or more subunits of the HCN channel and/or that may be treated by administering an effective amount of an agent that modulates the interaction between TRIP8b and one or more subunits of the HCN channel.

As used herein, the phrase “effective amount” shall mean that drug dosage that provides the specific pharmacological response for which the drug is administered in a significant number of subjects in need of such treatment. An effective amount of a drug that is administered to a particular subject in a particular instance will not always be effective in treating the conditions/diseases described herein, even though such dosage is deemed to be a therapeutically effective amount by those of skill in the art.

As used herein, the term “modulate” means decreasing or inhibiting activity and/or increasing or augmenting activity. For example, modulating the interaction between TRIP8b and one or more subunits of the HCN channel may mean decreasing or inhibiting the interaction and/or increasing or augmenting the interaction.

Chemical Entities

New chemical entities and uses for chemical entities are disclosed herein. The chemical entities may be described using terminology known in the art and further discussed below.

As used herein, an asterisk “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of an alkyl group (e.g., —(CH₂)ₙ— where n is an integer such as an integer between 1 and 20). An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxy” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively.

The term “cycloalkyl” refers to a monovalent saturated cyclic, bicyclic, or bridged cyclic (e.g., 11damantlyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloheteroalkyl” refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “cycloalkylene” refers to a cycloalkyl group that is unsaturated at one or more ring bonds.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3-to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “oxo” refers to a divalent oxygen atom —O—.

The term “carboxamido” as used herein refers to the radical —C(O)NRR′, where R and R′ may be the same or different. R and R′, for example, may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” or “amidyl” as used herein refers to a radical of the form —R¹C(O)N(R²)—, —R¹C(O)N(R²)R³—, —C(O)NR²R³, or —C(O)NH₂, wherein R¹, R² and R³, for example, are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers, or racemic mixtures. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. These compounds may be designated by the terms or by the terms ““tran”” or ““cis”” The present invention encompasses various stereoisomers of these compounds and mixtures thereof such as racemic mixtures. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

Inhibitors of TRIP8b and Uses Thereof

Disclosed herein are compounds which may be utilized as inhibitors of the interaction between the subunits of hyperpolarization-activated cyclic-nucleotide gated (HCN) channels, such as HCN1, and an auxiliary subunit of HCN channels which is the tetratricopeptide repeat-containing Rab8b-interacting protein (TRIP8b). The disclosed compounds may be used in pharmaceutical compositions and methods for treating neurological diseases and disorders such as depression, and in particular Major Depressive Disorder (MDD).

In some embodiments, the disclosed compounds have a formula I.

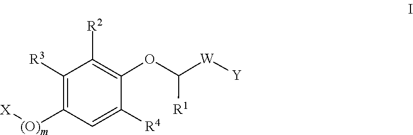

wherein:
X is phenyl optionally substituted at one or more positions with halogen, alkyl, alkoxy, hydroxyl, carboxamido, hydroxyl, cyano, nitro, haloalkyl, alkylthio, alkenyl, amino, alkylsulfonyl, hydroxyalkyl, or phenyl optionally substituted at one or more positions with halogen;
or X is pyrimidinyl optionally substituted at one or more positions with alkoxy;
or X is pyridinyl optionally substituted at one or more positions with halogen or haloalkyl;
or X is pyrazolyl optionally substituted at one or more positions with positions with alkyl or haloalkyl;
or X is thiophenyl optionally substituted at one or more positions with halogen;
or X is cycloalkyl;
or X is 1,4-benzodioxane;
or X is halogen or haloalkyl;
m is 0 or 1;
R¹ is H or alkyl (e.g., methyl or ethyl);
W is —CH₂— or —C(O)—;
Y is —NH-(Alk)ₙ-Z or —N(R⁵)—(Alk)ₙ-Z, wherein Alk is —CH₂— or —CH(CH₃)—, R⁵ is methyl or together with R⁴ forms a heterocycle, and n is 0-2;
or Y is

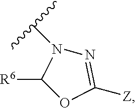

wherein R⁶ together with R⁴ forms a heterocycle;
or Y is —NH—S(O)₂—Z;
or Y is carboxyl, amino, alkylamino, dialkylamino, indolinyl optionally substituted at one or more positions with halogen; indanyl, 1,2,3,4-tetrahydroisoquinolinyl, or isoindolinyl;
Z is phenyl optionally substituted at one or more positions with halogen, alkyl, haloalkyl, aminosulfonyl, alkoxy, pyrazolyl, imidazolyl, alkylsufonyl, alkylaminocarbonyl, hydroxyl, cyano, nitro, alkenyl, aminoalkyl, or hydroxyalkyl;
or Z is 1,3-benzodioxole; piperidinyl; pyridinyl optionally substituted with alkoxy or benzothiazole; indolinyl optionally substituted with halogen or alkyl; cycloalkyl, or a cyclic ether;
R² is —CH₂—Het, wherein Het is a saturated heterocycle comprising 5, 6, or 7 atoms wherein at least one of the atoms is a nitrogen atom (e.g., piperazinyl, 1,4-diazepanyl, morpholinyl, piperidinyl, pyrrolidinyl), and the heterocycle is optionally substituted at one or more positions with alkyl, amino, alkylamino, dialkylamino, alkoxycarbonyl, or alkylsulfonyl;
or R² is H, —CH₂—NH₂, —C(O)—NH₂, —C(O)—OH, —C(NH₂)═N—OH, —CH₂—NH—CH₂—CH₂—N═C(NH₂)₂, —CH₂—NH—CH₂—CH₂—NH₂, —CH₂—NH—CH₂—CH₂—N(CH₃)₂, —CH₂—NH—CH₂—C(O)—NH₂, —CH₂—NH—CH₂—CH₂—NH—CH₃, —CH₂—NH—CH₂—CH₂—N═C(NH₂)₂, or —CH₂—NH—CH(NH₂)—N═NH;
or R₂ is

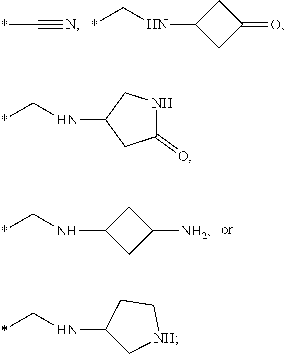

R³ is H or —CH₂—NH—CH₂—CH₂—N═C(NH₂)₂; and
R⁴ is H, —CH₂—NH₂, —C(O)—NH₂, or —C(NH₂)═N—OH.

In some embodiments of the disclosed compounds of formula I, X is phenyl optionally substituted at one or more positions with halogen, alkyl, alkoxy, hydroxyl, carboxamido, hydroxyl, cyano, nitro, haloalkyl, alkylthio, alkenyl, amino, alkylsulfonyl, hydroxyalkyl, or phenyl optionally substituted at one or more positions with halogen. In some embodiments, X is phenyl substituted at one or more positions with halogen. In further embodiments of formula I, X is phenyl substituted at the 4-position with halogen, alkyl, alkoxy, hydroxyl, carboxamido, hydroxyl, cyano, nitro, haloalkyl, alkylthio, alkenyl, amino, alkylsulfonyl, or hydroxyalkyl.

In some embodiments of the disclosed compounds of formula I, X is phenyl substituted at the 4-carbon position with F or Cl. In further embodiments of the disclosed compounds of formula I, X is 3,4-dichlorophenyl.

In the disclosed compounds of formula I, m is 0 or 1. In some embodiments of the disclosed compounds of formula I, m is 0.

In the disclosed compounds of formula I, R¹ is H or alkyl such as methyl or ethyl. In some embodiments of the disclosed compounds of formula I, R¹ is H.

In the disclosed compounds of formula I, W is —CH2- or —C(O)—. In some embodiments of the disclosed compounds, W is —C(O)—.

In some embodiments of the disclosed compounds of formula I, Y is —NH-(Alk)ₙ-Z or —N(CH₃)—(Alk)ₙ-Z, wherein Alk is —CH₂— or —CH(CH₃)—, and n is 0-2. In further embodiments of the compounds of formula I, Y is —NH—CH₂—Z or —NH—CH(CH₃)—Z.

In some embodiments of the disclosed compounds of formula I, Z is phenyl optionally substituted at one or more positions with halogen, alkyl, haloalkyl, aminosulfonyl, alkoxy, pyrazolyl, imidazolyl, alkylsufonyl, alkylaminocarbonyl, hydroxyl, cyano, nitro, alkenyl, aminoalkyl, or hydroxyalkyl. In further embodiments of the disclosed compounds of formula I, Z is phenyl substituted at one or more positions with halogen.

In some embodiments of the disclosed compounds of formula I, Z is phenyl substituted at the 4-carbon position with Cl or F. In further embodiments of the disclosed compounds of formula I, Z is 4-chlorophenyl.

In some embodiments of the disclosed compounds of formula I, R² is —CH₂—Het, wherein Het is a saturated heterocycle comprising 5, 6, or 7 atoms wherein at least one of the atoms is a nitrogen atom (e.g., piperazinyl, 1,4-diazepanyl, morpholinyl, piperidinyl, pyrrolidinyl), and the heterocycle is optionally substituted at one or more positions with alkyl, amino, alkylamino, dialkylamino, alkoxycarbonyl, or alkylsulfonyl. In further embodiments of the disclosed compounds of formula I, R² is —CH₂—Het, wherein Het is piperazinyl or piperidinyl, optionally substituted at one or more positions with alkyl, amino, alkylamino, dialkylamino, alkoxycarbonyl, or alkylsulfonyl. In even further embodiments of the disclosed compounds of formula I, R² is —CH₂-4-methylpiperazinyl or —CH₂-4-methylpiperidinyl.

In some embodiments of the disclosed compounds of formula I, R² is H, —CH₂—NH₂, —C(O)—NH₂, —C(O)—OH, —C(NH₂)═N—OH, —CH₂—NH—CH₂—CH₂—N═C(NH₂)₂, —CH₂—NH—CH₂—CH₂—NH₂, —CH₂—NH—CH₂—CH₂—N(CH₃)₂, —CH₂—NH—CH₂—C(O)—NH₂, —CH₂—NH—CH₂—CH₂—NH—CH₃, —CH₂—NH—CH₂—CH₂—N═C(NH₂)₂, or —CH₂—NH—CH(NH₂)—N═NH. In even further embodiments of the disclosed compounds of formula I, R² is —CH₂—NH₂, —C(O)—NH₂, —C(O)—OH, —C(NH₂)═N—OH, —CH₂—NH—CH₂—CH₂—N═C(NH₂)₂, —CH₂—NH—CH₂—CH₂—NH₂, —CH₂—NH—CH₂—CH₂—N(CH₃)₂, —CH₂—NH—CH₂—C(O)—NH₂, —CH₂—NH—CH₂—CH₂—NH—CH₃, —CH₂—NH—CH₂—CH₂—N═C(NH₂)₂, or —CH₂—NH—CH(NH₂)—N═NH.

In the disclosed compounds of formula I, R³ is H or —CH₂—NH—CH₂—CH₂—N═C(NH₂)₂. In some embodiments of the disclosed compound of formula I, R³ is H.

In the disclosed compounds of formula I, R⁴ is H, —CH₂—NH₂, —C(O)—NH₂, or —C(NH₂)═N—OH. In some embodiments of the disclosed compound of formula I, R⁴ is H.

Also disclosed herein are pharmaceutical composition comprising a compound of formula I, and a suitable pharmaceutical carrier, diluent, or excipient. The disclosed pharmaceutical compositions may comprise an effective amount of a compound of formula I for inhibiting TRIP8b activity in a subject to which the pharmaceutical composition is administered. The disclosed pharmaceutical compositions may comprise an effective amount of a compound of formula I for treating a disease or disorder associated with TRIP8b activity in a subject to which the pharmaceutical composition is administered. The disclosed pharmaceutical compositions may be formulated for use in treating diseases and disorders which include neurological diseases and disorders such as depression and particularly Major Depressive Disorder (MDD).\

Also disclosed herein are methods for treating diseases and disorders associated with TRIP8b activity in a subject in need thereof. The methods may comprise administering an effective amount of a compound of formula I for inhibiting TRIP8b activity in the subject. Diseases and disorders treated by the disclosed methods may include neurological diseases or such as depression and particularly Major Depressive Disorder (MDD).

The disclosed compounds may exhibit one or more biological activities. In particular, the disclosed compounds may modulate the interaction between TRIP8b and one or more subunits of the HCN channel. Screening assays to identify small-molecules that are capable of disrupting the TRIP8b-HCN1 interaction have been disclosed in the art. (See Han et al., “Method for Identifying Small Molecule Inhibitor of the Protein-Protein Interaction Between HCN1 and TRIP8b,” J. Vis. Exp. Nov 11; (117):54540, the content of which is incorporated herein by reference in its entirety). In some embodiments, the disclosed compounds may disrupt the TRIP8b-HCN1 at a concentration of less than about 100 μM, 50 μM, 10 μM, 1 μM, 0.1 μM, 0.05 μM, 0.01 μM, 0.005 μM, 0.001 μM, or less. Concentration ranges also are contemplated herein, for example, a concentration range bounded by end-point concentrations selected from 100 μM, 50 μM, 10 μM, 1 μM, 0.1 μM, 0.05 μM, 0.01 μM, 0.005 μM, 0.001 μM.

The disclosed compounds preferably are not toxic to cells. In some embodiments, the disclosed compounds art not toxic to cells at a concentration of greater than about 0.001 μM, 0.005 μM, 0.01 μM, 0.5 μM, 0.1 μM, 1.0 μM, 10 μM, and 100 μM or higher. Concentration ranges also are contemplated herein, for example, a concentration range bounded by end-point concentrations selected from 0.001 μM, 0.005 μM, 0.01 μM, 0.5 μM, 0.1 μM, 1.0 μM, 10 μM, and 100 μM.

The compounds utilized in the methods disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The disclosed compounds and pharmaceutical compositions comprising the disclosed compounds may be administered in methods of treating a subject in need thereof. For example, in the methods of treatment a subject in need thereof may include a subject having neurological disease, disorder, or condition such as depression (e.g., mood disorders such as major depression, dysthymia, bipolar disorder, mood disorders related to another health condition, and substance-induced mood disorders).

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as end-points any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as end-points any of these disclosed dose levels (e.g., 500-2000 ng/kg body weight of the subject).

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™) Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules. In some embodiments, the compounds are formulated as a composition for administration orally (e.g., in a solvent such as 5% DMSO in oil such as vegetable oil).

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are preferably applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Disclosed herein are compounds which exhibit TRIP8b-HCN1c40 Alphascreen IC50 activity less than 1 micromolar, including NUCC-0201976, NUCC-0201977, NUCC-0201978, NUCC-0201979, NUCC-0201981, NUCC-0201983, NUCC-0201990, NUCC-0201991, NUCC-0201992, NUCC-0201995, NUCC-0201997, NUCC-0201998, NUCC-0201999, NUCC-0202000, NUCC-0202001, NUCC-0202002, NUCC-0202003, NUCC-0202004, NUCC-0202014, NUCC-0202016, NUCC-0202017, NUCC-0202026, NUCC-0202027, NUCC-0202028, NUCC-0202033, NUCC-0202046, NUCC-0202047, NUCC-0202049, NUCC-0202050, and NUCC-0202054.

Further disclosed herein are compounds which exhibit TRIP8b-HCN1c40 Alphascreen IC50 activity between 1 micromolar to 2 micromolar, including NUCC-0201621, NUCC-0201969, NUCC-0201973, NUCC-0201974, NUCC-0201975, NUCC-0201980, NUCC-0201986, NUCC-0201988, NUCC-0201989, NUCC-0201993, NUCC-0201996, NUCC-0202008, NUCC-0202009, NUCC-0202011, NUCC-0202013, NUCC-0202015, NUCC-0202018, NUCC-0202019, NUCC-0202020, NUCC-0202021, NUCC-0202023, NUCC-0202025, and NUCC-0202048.

Further disclosed herein are compounds which exhibit TRIP8b-HCN1c40 Alphascreen IC50 activity between 2 micromolar to 5 micromolar, including NUCC-0201610, NUCC-0201629, NUCC-0201709, NUCC-0201710, NUCC-0201711, NUCC-0201712, NUCC-0201713, NUCC-0201972, NUCC-0201982, NUCC-0201985, NUCC-0201987, NUCC-0201994, NUCC-0202007, NUCC-0202012, NUCC-0202022, NUCC-0202024, NUCC-0202029, NUCC-0202031, NUCC-0202032, and NUCC-0202034.

Further disclosed herein are compounds which exhibit TRIP8b-HCN1c40 Alphascreen IC50 activity between 5 micromolar to 10 micromolar, including NUCC-0200590, NUCC-0201188, NUCC-0201229, NUCC-0201600, NUCC-0201604, NUCC-0201607, NUCC-0201608, NUCC-0201614, NUCC-0201615, NUCC-0201616, NUCC-0201617, NUCC-0201618, NUCC-0201625, NUCC-0201628, NUCC-0201971, NUCC-0202005, NUCC-0202006, and NUCC-0202010.

Further disclosed herein are compounds which exhibit TRIP8b-HCN1c40 Alphascreen IC50 activity between 10 micromolar to 20 micromolar, including NUCC-0201103, NUCC-0201187, NUCC-0201598, NUCC-0201599, NUCC-0201601, NUCC-0201602, NUCC-0201609, NUCC-0201620, NUCC-0201622, NUCC-0201624, NUCC-0202030, and NUCC-0202053.

Further disclosed herein are compounds which exhibit TRIP8b-HCN1c40 Alphascreen IC50 activity between 20 micromolar to 40 micromolar, including NUCC-0201186, NUCC-0201605, NUCC-0201606, NUCC-0201619, and NUCC-0201623.

### Examples

The following Examples are illustrative and should not be interpreted to limit the scope of the claimed subject matter.

In silico screening. The ZINC database (Irwin and Shoichet, 2005) was subjected to a panel of PAINS substructures filters with Smiles ARbitrary Target Specifications (SMARTS) strings to eliminate promiscuous and non-drug-like molecules that interfere with functionality of the target proteins (Baell and Holloway, 2010). Filtering resulted in a set of approximately 11.2 million commercially available compounds for further screening. The curated data set was then subjected to the LigPrep module of Schrödinger in OPLS3 force field at pH 7.4±1 retaining the specific chirality to produce a low energetic 3D structure for each molecule.

The protein preparation engine implemented in the Schrödinger software suite was utilized to prepare the protein for small molecule docking simulations. Analysis of the atomic structure of TRIP8b (pdb code 4EQF) which is complexed with an HCN2 carboxy-terminus peptide showed that the critical interactions between the peptide and TRIP8b included E67, N99, R236, N205, R206, N213, and N240 residues of TRIP8b. The Prime protein energy minimization module implemented in the Schrödinger suite was used to correct irrelevant side chains, add missing atoms, eliminate partial occupant rotamers, fix the undesired orientation of Asn, Gln and His residues, and to replace the “b” values by the optimized potential for liquid simulations (OPLS3) charges. The Glide (Halgren et al., n.d.) docking engine is a built using a grid-based algorithm and hence, a 12×12×12 Å grid cube was generated which contained the above-mentioned critical residues.

For the virtual screening, we used the curated library of approximately 11.2 million drug-like compounds described above and the OPLS3 force field. The ligand van der Waals radii was scaled to 0.80 Å with partial atomic charges <0.15 esu and the three-tier Glide docking algorithm was executed (Anon, n.d.). The output of this three-tier docking engine was analyzed using the XP-visualization tools by considering the interactions of the compounds with the critical residues reported above. We selected the 77 compounds having a Glide docking score <−6.0 (Anon, n.d.). We then used an orthogonal docking engine (Surflex) implemented in the Sybyl interface of Tripos (Pham and Jain, n.d.) to obtain consensus binding poses and to enrich the hit rate using alternative flexible ligand docking tools (Cross et al., n.d.). We found 52 compounds that showed similar binding poses with comparable binding scores by comparing the binding poses and scores obtained from both docking experiments. On the basis of commercial availability and synthetic tractability, we purchased 30 of these compounds for testing.

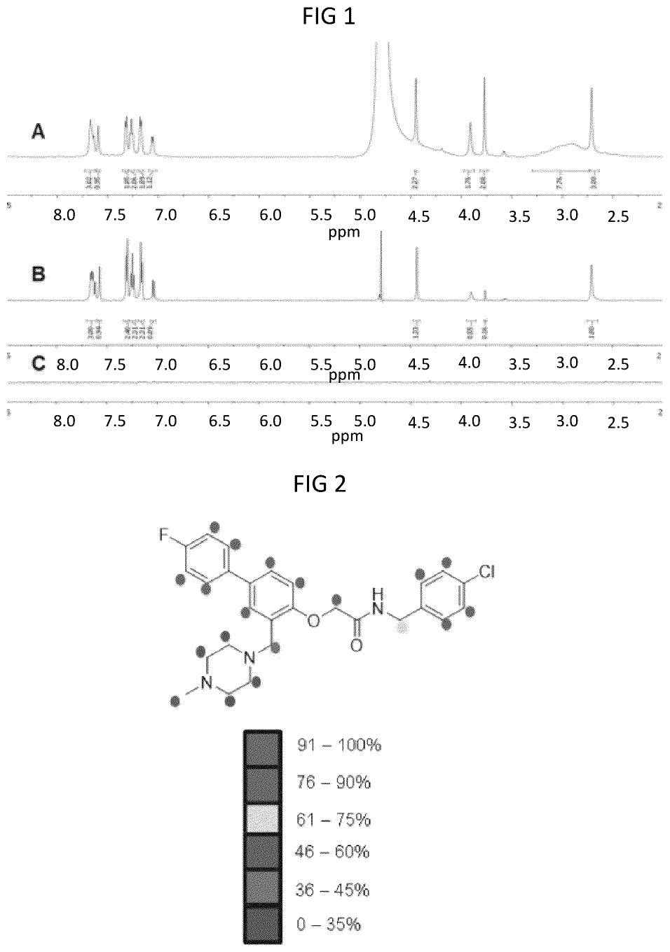

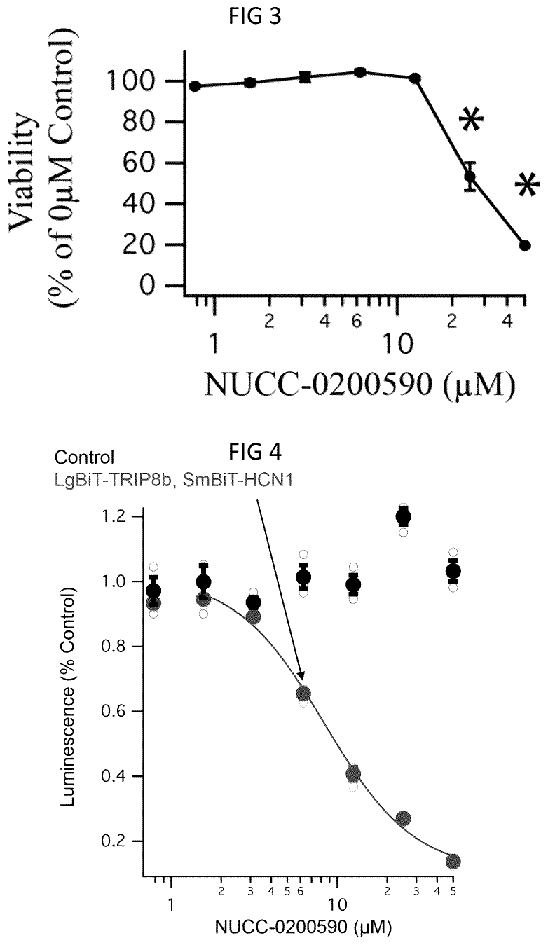

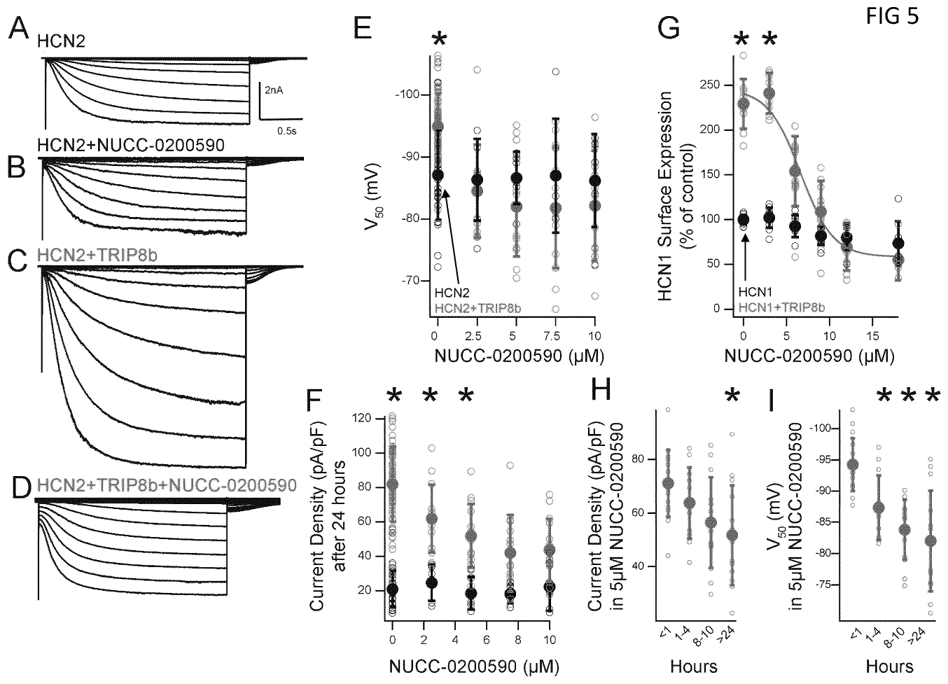

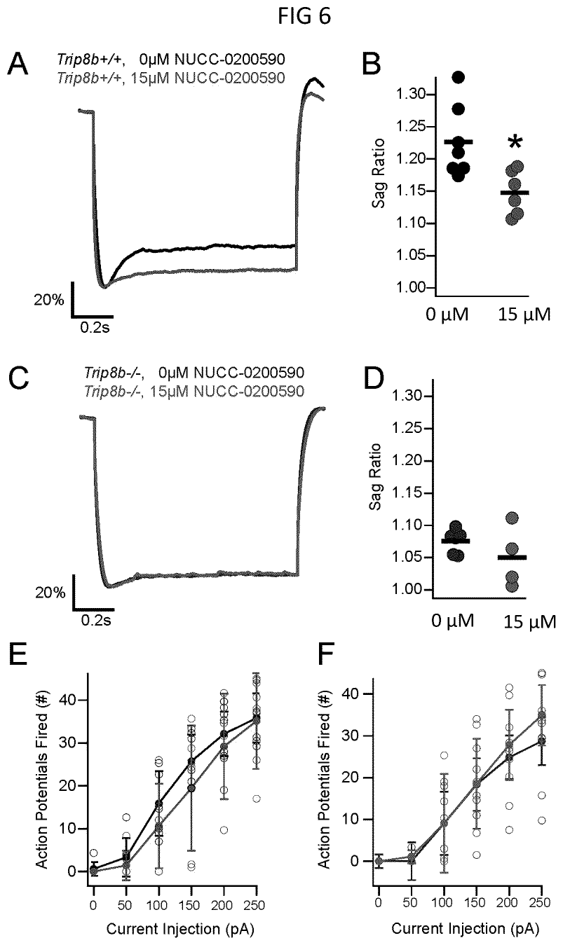

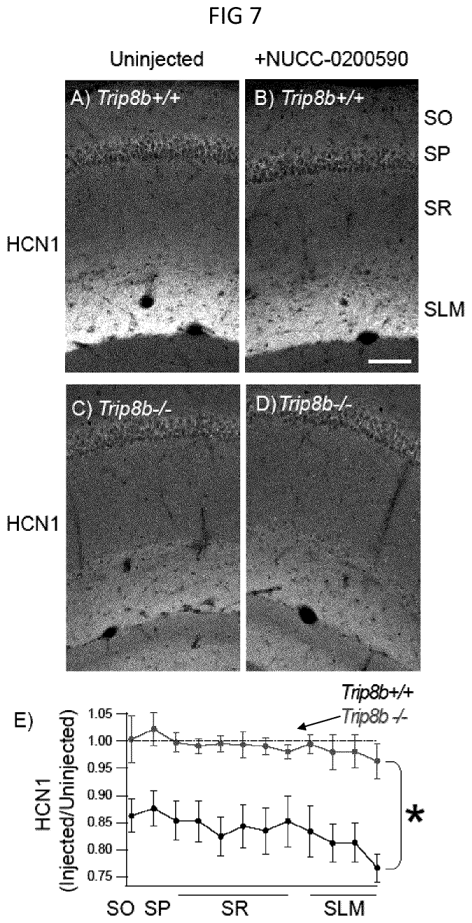

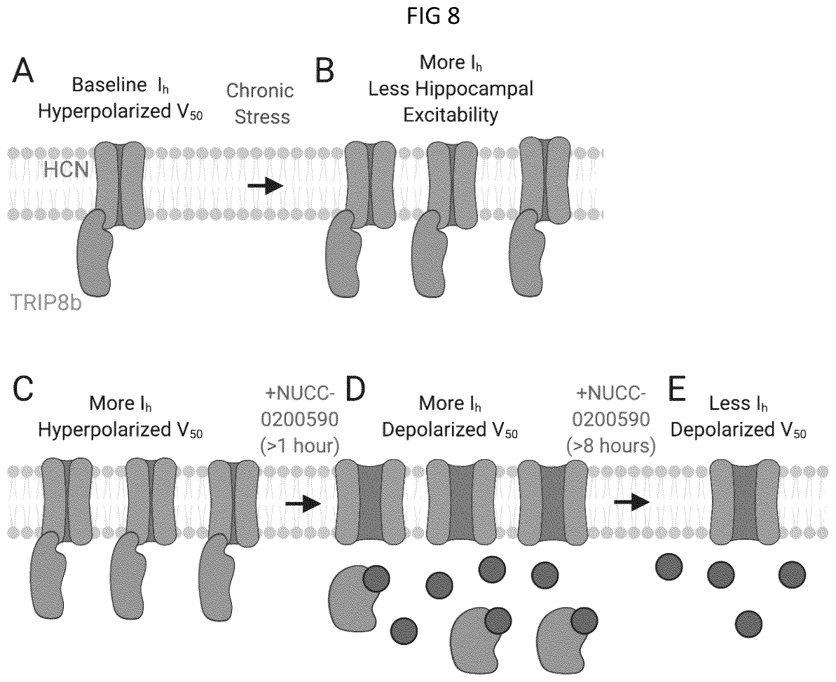

Biochemical screening. Potential hits obtained from the above-described in silico screen were tested in biochemical assays to characterize biological activity. The molecular mechanism of binding of HCN to TRIP8b has been elucidated by determination of the co-crystal structure of the carboxyl-terminal SNL sequence (SRLSSNL) of HCN2 bound to TRIP8b (Bankston et al., 2012). In the AlphaScreen assay, the most potent hit compound (NUCC-0200590) had an IC50 value of 19.9±0.9 μM (FIG. 1).

Saturation Transfer Difference (STD) NMR. In order to validate and gain structural understanding of the binding mode of NUCC-0200590 to TRIP8b, we performed STD-NMR experiments on the compound in the presence of TRIP8b. STD-NMR is a popular ligand-observed NMR technique used to study protein-ligand interactions of weak affinity ligands (high nM to low mM) range. It is a robust technique because it relies on the measurement of relatively simple 1H-NMR signals of the ligand instead of the complex spectrum of the protein. This technique is based on the transfer of saturation from the protein to the bound ligand when the protein is selectively irradiated at a frequency where only resonances from the protein are located. The fast exchange of ligands with the binding sites allows for identification of the epitope of the protein most closely bound by the ligand. The STD-NMR of NUCC-0200590 bound to TRIP8b was carried out using a 20-fold ligand excess (LE) with respect to the protein at on-resonance frequency of −1 ppm and off-resonance frequency of 40 ppm. In order to better define the binding epitope, a 100-fold LE was used (20 μM protein:2 mM ligand) in the STD-NMR experiment (FIG. 2). The 1H-NMR of 2 mM ligand in deuterated PBS at pH 7.4 showed the entire proton signals (FIG. 1a). On irradiating a solution of NUCC-0200590 (2 mM) and TRIP8b (20 μM), there was clear evidence of saturation transfer. A control STD-NMR experiment of the ligand without the protein showed no signal as there was no saturation transfer occurring due to the absence of TRIP8b (FIG. 1c). Analysis of the saturation transfer effects of the ligand showed that the aromatic protons produced the highest signal. Interestingly, the aliphatic protons of the piperazine showed no STD effect which is in agreement with the docked pose of NUCC-0200590 which shows this portion oriented towards the solvent. However, the methyl group on the piperazine showed a significant STD signal, suggesting that the piperazine might not be entirely solvent exposed (FIG. 2).

Solubility and Mouse Microsomal Stability. Attempts to use the free base of NUCC-0200590 directly in saturation transfer difference NMR (STD-NMR) and surface plasmon resonance (SPR) were complicated due to the compound's poor aqueous solubility, requiring 35% DMSO in PBS (pH 7.4) to see protons signal in 1H-NMR (above). In order to improve the aqueous solubility to support additional biochemical assays we made and characterized several salt forms. The formic acid (FA) salt was recovered after reverse-phase preparative HPLC while using formic acid as a modifier. Though this had better aqueous solubility than the free base, it still required 15% DMSO in PBS to see the proton signals in 1H-NMR. On stirring the free amine in 4M HCl in dioxane, the compound was converted to the HCl salt as a white solid (Scheme 1). Comparison of the thermodynamic solubility of the 3 forms (free amine, FA salt and HCl salt) of NUCC-0200590 performed with an in-house protocol (Iyamu et al., 2019) showed that the free amine was completely insoluble in PBS at pH 7.4 and room temperature after stirring for 16 hours. The FA salt showed much improved solubility (45 μM) in PBS at pH 7.4 although the HCl-salt showed greater thermodynamic solubility (252 μM). As a result, the HCl-salt of NUCC-0200590 was used for biophysical assays. The inhibitor was also screened for in vitro stability towards mouse liver microsomes to evaluate its potential metabolism and suitability for future in vivo experiments. The microsomal stability was measured after a 60-minute incubation at 37° C. with mouse liver microsomes. The compound was almost completely consumed by mouse liver microsomes after a 60-minute incubation as only 3.7% of the parent compound remained.

Compounds of the present disclosure may be prepared according to Scheme 1.

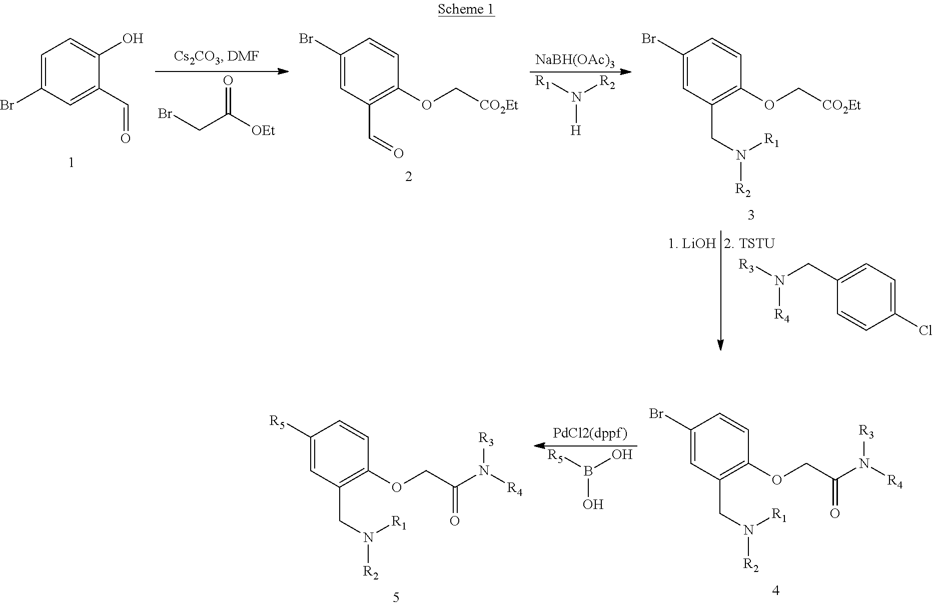

Starting with 5-bromo-2-hydroxybenzaldehyde (1) can be O-alkylated with an inorganic base such as Cs₂CO₃ and an alkyl halide such as ethyl 2-bromoacetate to provide the phenoxyacetate (2). The aldehyde can then be subjected to reductive amination with primary or secondary amines using NaBH(OAc)₃ or other reducing agents. This provides the aminomethyl phenyl derivatives (3). Hydrolysis of the ester using a base such as LiOH followed by amide coupling with primary or secondary amines and peptide coupling reagents, e.g., TSTU, EDCI, HBTU gives the secondary or tertiary amide (4). Finally, Suzuki coupling with a metal catalyst such as PdCl₂(dppf) and an appropriate boronic acid or ester gives the final compounds (5).

An exemplary synthetic route for compound NUCC-0200590 is provided in Scheme 2. Those of ordinary skill will recognize that compounds of the present disclosure may be prepared by similar methods by choosing appropriate starting material.

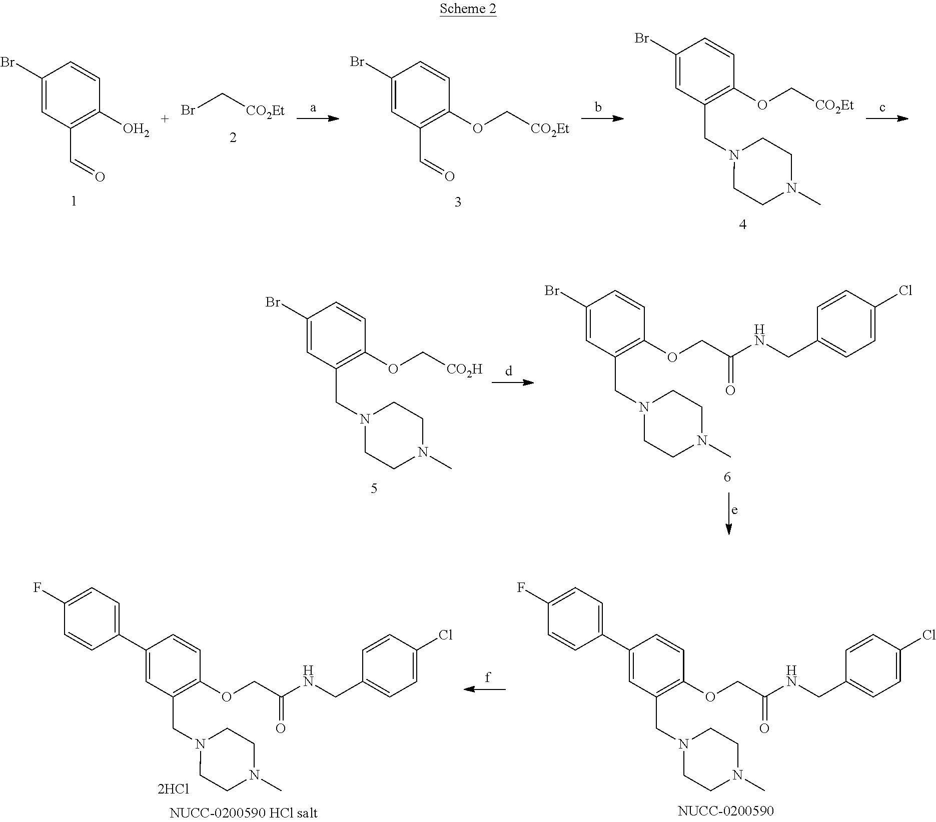

Alkylation of commercially available 5-bromosalicylaldehyde (1) with ethyl bromoacetate (2) afforded acetate 3 in quantitative yield. Reductive amination with N-methyl piperazine afforded 4 which was hydrolyzed under basic condition to the carboxylic acid 5. Amide coupling of acid 5 with (4-chlorophenyl)methanamine using HOBt and EDCI was not successful so alternative approaches were explored. Though a number of early transition-metal complexes, especially from zirconium and titanium, have been used as catalysts in direct coupling of deactivated acids in high yields, the conversion was very low in our case. Conversion of the acid to an acid chloride before amidation was successful albeit with low yield. A larger set of coupling reagents were screened and O—(N-Succinimidyl)-N,N,N′,N′-tetramethyluronium tetrafluoroborate (TSTU) was identified as the best coupling reagent and produced the acetamide 6 in 66% yield. Suzuki coupling with (4-fluorophenyl)boronic acid afforded the final compound NUCC-0200590 in excellent yield. The final compound was converted to the formic acid (FA) salt by exposure to the FA modifier in prep HPLC purification or to the hydrochloric acid (HCl) salt by treating the free base with HCl in dioxane.

Unless otherwise noted, all materials for the synthetic chemistry portion were obtained from commercial suppliers (Combi-Blocks, CombiPhos, Fisher Scientific, Sigma-Aldrich, or VWR) and used without further purification. The 1H-and 13C-NMR spectra were recorded on a Bruker AVANCE 500 MHz spectrometer using CDCl3, CD3OD or D2O as the solvent. Chemical shifts are expressed in ppm (δ scale) and referenced to residual protonated solvent. When peak multiplicities are reported, the following abbreviations are used: s (singlet), d (doublet), t (triplet), q (quartet), m (multiplet), or br s (broad singlet). Thin layer chromatography (TLC) was performed on glass backed Merck silica gel 60 F254 plates, column chromatography was performed using KP-SIL silica gel (Biotage, USA), and flash column chromatography was performed on Biotage prepacked columns using the automated flash chromatography system Biotage Isolera One. Low resolution liquid chromatography/mass spectrometry (LCMS) was performed on a Waters Acquity-H UPLC/MS system with a 2.1 mm×50 mm, 1.7 μm, reversed phase BEH C18 column and LCMS grade solvents. A gradient elution from 95% water+0.1% formic acid/5% acetonitrile+0.1% formic acid to 95% acetonitrile+0.1% formic acid/5% water +0.1% formic acid over 2 min plus a further minute continuing this mixture at a flow rate of 0.85 mL/min was used as the eluent. Total ion current traces were obtained for electrospray positive and negative ionization (ESI+/ESI−). The purities of all the final compounds were of >95% as determined by UPLC analysis unless otherwise indicated. High Resolution Mass analysis was performed on Agilent 6210A LC-TOF. Preparative HPLC Purification was carried out using a Gilson GX-271 preparative scale reverse phase HPLC system with Gilson model 159 UV-VIS detector and Phenonemex Kinetex 5 m, C18, 100 A, 50 mm×30 mm column. Compounds were eluted using a gradient elution of A:B (90:10 to 0:100) over 5 min at a flow rate of 50.0 mL/min, where solvent A was H2O (with 0.1% formic acid) and solvent B was CH3CN (with 0.1% formic acid). The product fractions were combined and dried using a Genevac EZ-2 Centrifugal Evaporator.

ethyl 2-(4-bromo-2-formylphenoxy)acetate (3). Was synthesized according to Org. Lett., 2015, 17 (23), pp 5824-5827.

ethyl 2-(4-bromo-2-((4-methylpiperazin-1-yl)methyl)phenoxy)acetate (4). A mixture of ethyl 2-(4-bromo-2-formylphenoxy)acetate (0.70 g, 2.44 mmol), 1-methylpiperazine (0.295 ml, 2.68 mmol) and acetic acid (0.140 ml, 2.44 mmol) in 1,2-dichloroethane (10 ml) was stirred at rt for 30 mins, then sodium triacetoxyhydroborate (0.62 g, 2.93 mmol) was added and stirring was continued for 5 h. The reaction was diluted with DCM, quenched with NaBHCO3, extracted with DCM (10 mL×3). The organic layer was dried with Na2SO4, concentrated and was purified by Biotage eluting with 6% MeOH in DCM to afford 4 (0.73 g, 81%) as a colorless oil. 1H NMR (500 MHz, CDCl3) δ 7.26 (s, 1H), 7.01 (dd, J=8.7, 2.6 Hz, 1H), 6.37 (d, J=8.7 Hz, 1H), 4.35 (s, 2H), 3.98 (q, J=7.1 Hz, 2H), 3.34 (s, 2H), 2.58-2.26 (m, 4H), 2.26-2.08 (m, 4H), 2.03 (s, 3H), 1.02 (t, J=7.1 Hz, 3H). 13C NMR (126 MHz, CDCl3) δ 168.41, 155.15, 132.98, 130.37, 129.73, 114.07, 113.50, 65.92, 61.21, 55.40, 55.09, 52.89, 45.95, 14.10. LCMS (ESI) m/z: [M+H]+ 371.3.

2-(4-bromo-2-((4-methylpiperazin-1-yl)methyl)phenoxy)acetic acid (5). To a solution of ethyl 2-(4-bromo-2-((4-methylpiperazin-1-yl)methyl)phenoxy)acetate (0.62 g, 1.67 mmol) in THE (6 mL) and water (2 mL) was added lithium hydroxide monohydrate (0.21 g, 5.01 mmol) and the reaction was stirred at rt until complete while monitoring by TLC. The solvent was removed in vacuo to yield a crude solid which contained the lithium salt. The crude solid was dissolved in DCM, filtered and the filtrate was purified by prep HPLC and concentrated to afford 5 (0.57 g, 100%) as a white fluffy solid. 1H NMR (500 MHz, CDCl3) δ 10.56 (s, 2H, COOH), 8.29 (s, 2H, FA), 7.33 (d, J=8.7 Hz, 1H), 7.22 (s, 1H), 6.74 (d, J=8.6 Hz, 1H), 4.50 (s, 2H), 3.50 (s, 2H), 3.23-2.77 (m, 5H), 2.79-2.64 (m, 4H), 2.61 (s, 3H). 13C NMR (126 MHz, CDCl3) δ 172.28, 166.94, 155.23, 134.55, 132.42, 127.20, 114.04, 113.95, 67.19, 57.15, 52.86, 50.25, 43.26. LCMS (ESI) m/z: [M+H]+ 343.3.

2-(4-bromo-2-((4-methylpiperazin-1-yl)methyl)phenoxy)-N-(4-chlorobenzyl)acetamide (6). To a solution of 2-(4-bromo-2-((4-methylpiperazin-1-yl)methyl)phenoxy)acetic acid (0.30 g, 0.87 mmol) and (4-chlorophenyl)methanamine (0.13 ml, 1.05 mmol) in DMF (4 mL) was added 2-(2,5-dioxopyrrolidin-1-yl)-1,1,3,3-tetramethyluronium tetrafluoroborate (0.53 g, 1.75 mmol) and N-ethyl-N-isopropylpropan-2-amine (0.46 mL, 2.62 mmol). The reaction was stirred at rt overnight. The reaction was quenched with water and extracted with EA (20 mL×3). The organic layers were combined, washed with brine, dried with Na2SO4 and concentrated in vacuo. The crude was purified by prep HPLC and concentrated to afford 6 (0.27 g, 66%) as a yellow solid. 1H NMR (500 MHz, CDCl3) δ 8.24 (s, 1H, FA), 7.46 (d, J=8.7 Hz, 1H), 7.36 (s, 1H), 7.33-7.26 (m, 2H), 6.99 (dd, J=8.4, 3.6 Hz, 2H), 6.88-6.81 (m, 1H), 4.73 (d, J=3.3 Hz, 2H), 4.62-4.53 (m, 2H), 3.49 (d, J=3.5 Hz, 2H), 2.65-2.41 (m, 4H), 2.41-2.04 (m, 7H). 13C NMR (126 MHz, Chloroform-d) δ 168.89, 155.09, 136.79, 134.55, 133.13, 132.01, 128.81, 128.22, 113.92, 113.45, 67.19, 58.53, 54.80, 53.47, 45.93, 41.95. LCMS (ESI) m/z: [M+H]+ 457.3.

N-(4-chlorobenzyl)-2-((4′-fluoro-3-((4-methylpiperazin-1-yl)methyl)-[1,1′-biphenyl]-4-yl)oxy)acetamide (NUCC-0200590). To a solution of 2-(4-bromo-2-((4-methylpiperazin-1-yl)methyl)phenoxy)-N-(4-chlorobenzyl)acetamide (0.18 g, 0.38 mmol), (4-fluorophenyl)boronic acid (0.08 g, 0.57 mmol) and K2CO3 (0.16 g, 1.13 mmol) in dioxane (2 mL) and water (1 mL) was added PdCl2(dppf) (0.03 g, 0.04 mmol) and degassed with N2 for 5 mins. The reaction vial was sealed and heated to 100° C. for 1 h. The reaction was cooled to rt and extracted with EA (5 mL×3). The organic layers were combined, washed with brine, dried with Na2SO4 and concentrated in vacuo. The crude was purified with Biotage eluting with 50% EA in hexanes to afford NUCC-0200590 (0.13 g, 73%) as a thick yellow oil. 1H NMR (500 MHz, CD3OD) δ 7.63-7.54 (m, 4H), 7.29-7.25 (m, 2H), 7.21-7.17 (m, 2H), 7.15 (d, J=8.7 Hz, 2H), 7.10 (d, J=9.2 Hz, 1H), 4.78 (s, 2H), 4.47 (s, 2H), 3.89 (s, 2H), 2.98-2.77 (m, 4H), 2.77-2.58 (m, 4H), 2.45 (s, 3H). 13C NMR (126 MHz, CD3OD) δ 171.45, 163.67 (d, J=249.89 Hz), 157.23, 138.50, 137.66 (d, J=3.21 Hz), 135.29, 134.05, 131.54, 130.01, 129.64, 129.51 (d, J=7.23 Hz), 129.34, 116.57 (d, J=23.46 Hz), 114.27, 68.53, 57.72, 54.42, 52.61, 44.88, 43.01. HRMS m/z calcd for C22H29F3N40 [M+H]+: 481.1940; found: 481.1937.

N-(4-chlorobenzyl)-2-((4′-fluoro-3-((4-methylpiperazin-1-yl)methyl)-[1,1′-biphenyl]-4-yl)oxy)acetamide formate (NUCC-0200590 formic acid salt). The crude of the free NH2 of NUCC-0200590 was subjected to purification by prep HPLC and concentrated to give the FA salt. 1H NMR (500 MHz, CD3OD) δ 8.52 (s, 1H, FA), 7.61-7.53 (m, 4H), 7.26 (d, J=8.4 Hz, 2H), 7.18 (d, J=8.5 Hz, 2H), 7.14 (d, J=8.7 Hz, 2H), 7.10-7.06 (m, 1H), 4.77 (s, 2H), 4.47 (s, 2H), 3.86 (s, 2H), 2.92-2.76 (m, 4H), 2.76-2.60 (m, 4H), 2.44 (s, 3H). 13C NMR (126 MHz, CD3OD) δ 171.42, 163.74 (d, J=248.18 Hz), 157.21, 138.51, 137.68 (d, J=3.17 Hz), 135.25, 134.04, 131.51, 129.98, 129.64, 129.50 (d, J=8.22 Hz), 129.26, 125.87, 116.56 (d, J=21.85 Hz), 114.22, 68.54, 57.72, 54.44, 52.57, 44.82, 42.98.

N-(4-chlorobenzyl)-2-((4′-fluoro-3-((4-methylpiperazin-1-yl)methyl)-[1,1′-biphenyl]-4-yl)oxy)acetamide hydrochloride (NUCC-0200590 HCl Salt). To a solution of NUCC-0200590 free base (0.10 g, 0.21 mmol) in dioxane was added 4M HCl in dioxane (0.2 mL, 0.62 mmol). The solution was stirred at room temperature for 1 h. The solution was concentrated and triturated with ether to afford NUCC-0200590 (HCl Salt) (0.11 g, 94%) as a white solid. 1H NMR (500 MHz, 126 MHz, D2O) δ 7.76 (dd, J=8.6, 2.4 Hz, 1H), 7.70-7.63 (m, 3H), 7.33-7.29 (m, 2H), 7.29-7.24 (m, 2H), 7.19 (dd, J=8.7, 3.2 Hz, 3H), 4.96 (s, 2H), 4.48 (s, 2H), 4.43 (s, 2H), 3.78-3.29 (m, 8H), 2.96 (s, 3H). 13C NMR (126 MHz, D2O) δ 170.53, 162.97, 161.02, 155.36, 136.58, 134.77 (d, J=2.95 Hz), 133.64, 132.14, 131.21, 129.89, 128.60, 128.21, 128.08 (d, J=8.09 Hz), 117.74, 115.49 (d, J=23.39 Hz), 113.02, 67.09, 55.79, 50.09, 48.50, 42.84, 42.04.

STD-NMR. STD NMR Spectrum was recorded using a Bruker Advanced 500 spectrometer equipped with a Cryogenic probe and processed by Bruker® Topspin software. An experiment to confirm TRIP8b binding of the inhibitor consisted of compound NUCC-0200590 (100 μM) and TRIP8b(1a-4) (5 μM) in 5% DMSO-d6 and deuterated PBS, pH 7.4 buffer (600 μl total volume). The prepared solution was vortexed for 30 secs and submitted for NMR. The epitope mapping experiment was performed the same way, except using 2 mM NUCC-0200590 and 20 μM TRIP8b to increase signal to noise. STD NMR experiments were performed with a train of 50 ms Gaussian-shaped saturating pulses at 200 Hz power for 2 s with “on” resonance saturation at −1 ppm and “off” resonance saturation at 40 ppm. The relaxation delay was 2 s before the saturating pulses. The number of scans was 2048 and the spectral width was 10 ppm.

Cellular Assays. The ability of NUCC-0200590 to disrupt the TRIP8b-HCN interaction in cell-based assays was tested using the FA version of the compound for the remainder of the paper. There are many isoforms of TRIP8b, which differ in terms of the N terminal exons that are included (with each isoform named by which exons are included, see (Lewis et al., 2009; Han et al., 2020). As an exemplary TRIP8b isoform, we exclusively used the 1a-4 isoform, which is thought to be the most commonly occurring isoform in the brain (Lewis et al., 2009; Han et al., 2020). All experiments described below use this isoform so that ‘TRIP8b’ specifically refers to ‘TRIP8b(1a-4)’. Similarly, although HCN1 and HCN2 are thought to bind to TRIP8b in precisely the same manner, HCN1 and HCN2 have slightly different electrophysiological properties (Robinson and Siegelbaum, 2003). As such, we alternate between the two HCN isoforms in order to confirm our results for both subunits in the experiments below (as outlined).

Cell Viability Assays. HEK293T Cells (ATCC, Manassas, Virginia) were maintained as previously described (Lyman et al., 2017b). These cells were plated at 30-50% confluence and incubated for 24 hours with the concentration of NUCC-0200590 specified in the main text. The cells were then incubated with alamar Blue assay reagent per the Manufacturer's instructions (ThermoFisher, Rockford, IL) prior to reading the plates on a plate reader.

Flow cytometry. The surface expression evaluation by flow cytometry was performed as described previously (Han et al., 2011). HEK293 cells were transfected with HA-HCN1 and TRIP8b(1a-4) or pEGFP (as a control). Cells were incubated with varying concentrations (in uM: 3, 6, 9, 12, 18) of NUCC-0200590 or DMSO (as a control) for overnight. Nonpemeabilized cells were stained with mouse anti-HA primary antibody to label surface HA and then were stained with secondary antibody conjugated to Alexa-647 (Invitrogen). Cells were run in a Cyan ADP flow cytometer (Dakocytomation) at the Vanderbilt University Medical Center Flow Cytometry Core, and data were analyzed by FloJo software. The presented fluorescence index was calculated as the integral of the Alexa-647 flu-orescence with respect to cell number, which was normalized by eGFP-positive cells in each transfection condition. The fluorescence indices for each condition were normalized to the control transfection condition (HCN1 plus eGFP). Statistical analyses were performed using 1-way ANOVA with Tukey's post hoc-test.

HEK Electrophysiology. HEK cells stably expressing HCN2 (generated in our previous report (Foote et al., 2019)) were plated on autoclaved 12 mm diameter glass coverslips coated with poly-L-lysine (0.1 mg/mL), washed with standard Hank's Balanced Salt medium (without calcium or magnesium). Cells were transiently transfected 24 hours later with EGFP plus a control vector or TRIP8b(1a-4) using lipofectamine 2000 according to the manufacturer's instructions. Lipofectamine exposure was limited to 60 mins, followed by a rinse and replacement of pre-conditioned culture medium. Different doses of NUCC-0200590 were added in the medium following transfection. For time course studies, 5 uM of NUCC-0200590 was added in the medium. Whole cell recordings were performed 24-48 hours post transfection with pipettes made from borosilicate glass using a vertical puller (Narishige) with a final resistance of ˜3 MΩ. Cells were held at −40 mV in voltage clamp and stepped from −40 to −120 mV in 10 mV increments. Extracellular solution consisted of (in mM): 145 NaCl, 10 KCl, 10 glucose, 10 HEPES, 2 CaCl₂), 1 MgCl2, buffered to pH 7.4 and an osmolarity of 312-315 mOsm. Intracellular pipette solution contained 135 K-gluconate, 10 MgCl2, 0.1 CaCl₂), 1 EGTA, 10 HEPES, and 2 Mg-ATP buffered to pH 7.3 and an osmolarity of 295-305 mOsm. Maximal tail current amplitudes were fitted to the Boltzmann equation as described in (Santoro et al., 2011). Maximal Ih current density was calculated by dividing the maximal tail current by the cell capacitance, as described in (Bankston et al., 2012). Currents were recorded via WinWCP software (University of Strathclyde, Scotland, UK), a MultiClamp 700A amplifier (Molecular Devices), and a National Instruments USB6221 interface card. Sampling rate was 10 kHz. Series resistance was monitored throughout each experiment and cells were discarded if the series resistance rose during the experiment. Statistical analyses were performed using 1-way ANOVA with Tukey's post hoc-test.

Slice Electrophysiology. Whole cell recordings from CA1 pyramidal neurons were performed using an electrophysiology rig described previously (Zhou et al., 2013; 2015). 300 micron sagittal sections were made from male and female mice with 5 μM CGP52432, 2 μM SR-95531, and 10 μM MK-801 to block GABAB, GABAA, and NMDAR. ACSF was composed of 125 mM NaCl, 2.5 mM KCl, 25 mM NaHCO₃, 1.25 mM Na2PO4, 1 mM MgCl2, 2 mM CaCl₂), and 25 mM glucose while the internal solution was 115 mM K-gluconate, 20 mM KCl, 10 mM Na2Phosphocreatinine, 2 mM MgATP, and 0.3 mM NaGTP. NUCC-0200590 was incubated in the slice chamber in a subset of experiments as described in the text. Current clamp recordings were performed at a holding potential of −70 mV.

Immunohistochemistry. Immunohistochemistry was performed as described in a prior report (Han et al., 2016a). Briefly, primary antibodies used included anti-HCN1, anti-TRIP8b, and MAP2. Images were acquired using a confocal microscope at the Vanderbilt Cell Imaging Shared Resource supported by NIH grants CA68485, DK20593, DK58404, DK59637, and EY08126) and then analyzed using custom-written routines in MATLAB (Mathworks, Natick, MA). The analysis of immunohistochemical images was carried out identically to our previous report (Han et al., 2016a), and a blinded observer drew ROIs over the stratum oriens and stratum pyramidale. A larger ROI was also drawn encompassing the stratum lacunosum moleculare and stratum radiatum which was then subdivided into ten equally spaced ROIs in order to examine the distal dendritic enrichment of HCN channels. Schematic of TRIP8b mediated HCN channel trafficking was created using BioRender.com

NanoBiT live cell protein-protein interaction assay. HEK293T cells (obtained freshly from ATCC) were plated in DMEM (Thermo Fisher, #11965-092) with 10% FBS (Thermo Fisher, #16000-044) and Pen/Strep (Thermo Fisher #15140-122) at 1 million cells per well in 6-well tissue culture treated plates. The next day, cells were co-transfected using Lipofectamine 3000 with plasmids encoding the small Nanoluciferase fragment (SmBiT) fused to the N-terminus of full-length HCN1 (SmBiT-HCN1), and the large Nanoluciferase fragment (LgBiT) fused to the N-terminus of TRIP8b(1a-4) (LgBiT-TRIP8b). The plasmids were constructed by cloning into backbone vectors from the Promega NanoBiT MCS cloning kit #N2014. As a control for specific inhibition, other cells were co-transfected with SmBiT-PRKACA and LgBiT-PRKAR2A positive control plasmids from the kit. 24 hours post-transfection, the cells were trypsinized with TrypLE (Thermo Fisher, #12604-013), washed and suspended in phenol red-free Opti-MEM I (Thermo Fisher, #11058-021), and 25,000 cells per well were plated in white 384-well tissue culture-treated plates (Greiner #781080). A dilution series of NUCC-0200590 or DMSO vehicle control was added to each well and incubated for 30 minutes at 37° C. Luminescence was generated by adding Nano-Glo live cell substrate (Promega, #N2011) and incubating for 30 minutes at 37° C. prior to detecting on a luminescent reader (Biotek Neo2).

AlphaScreen Assay. 20 nM ofGST-tagged HCNlc40 protein (corresponding to the C terminal 40 amino acids of HCN1) was incubated with 200 nM of His-tagged TRIP8b(241-602) and varying concentrations of the test compound as described in our prior report (Han et al., 2015). After a 3 h incubation, anti-GST AlphaScreen acceptor beads were added for 1.5 h followed by AlphaScreen nickel chelate donor beads for 1.5 h. AlphaScreen signal was quantified using a PerkinElmer (Waltham, MA) EnSpire multimode plate reader.

TABLE 1
Representative compounds and their TRIP8b-HCN activity

| | | TRIP8b-HCN1c40 |
|---|---|---|
| | | Alphascreen IC₅₀ |
| Structure | Molecule Name | (μM) |
| 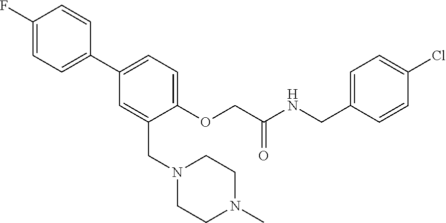 | NUCC-0200590 | 5.4 |
| 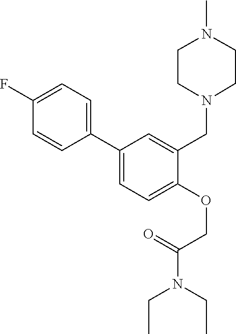 | NUCC-0201100 | 150 |
| 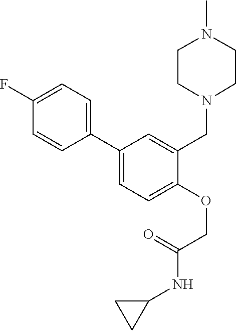 | NUCC-0201101 | 110 |
| 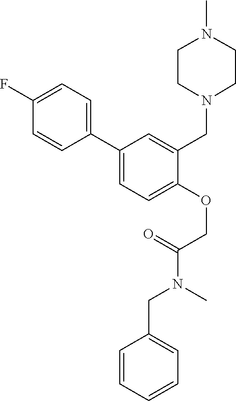 | NUCC-0201102 | 91 |
| 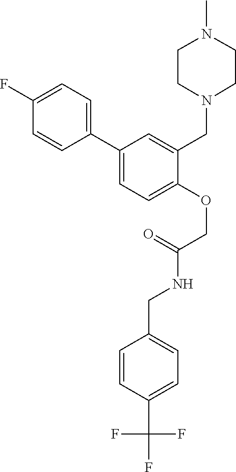 | NUCC-0201103 | 15 |
| 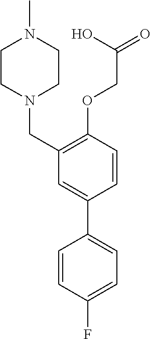 | NUCC-0201181 | NA |
| 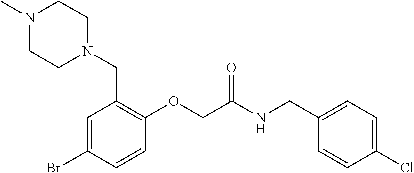 | NUCC-0201182 | 120 |
| 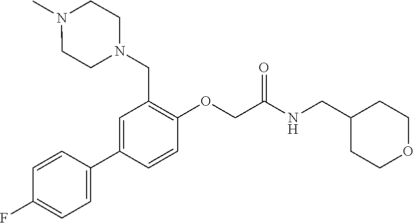 | NUCC-0201183 | NA |
| 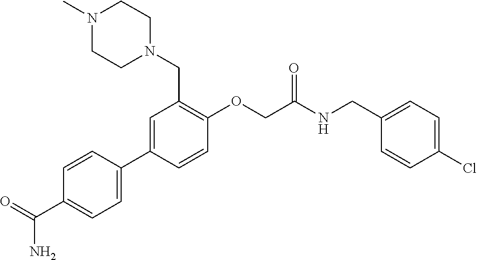 | NUCC-0201184 | 92 |
| 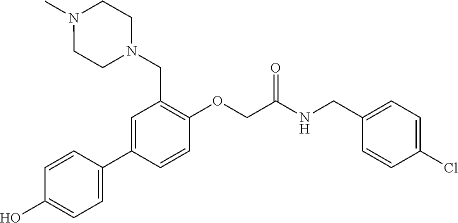 | NUCC-0201185 | 42 |
| 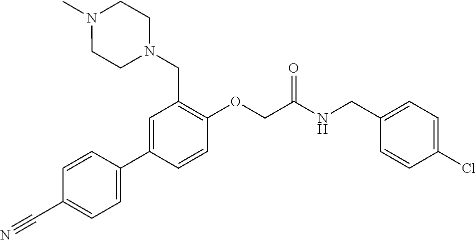 | NUCC-0201186 | 38 |
| 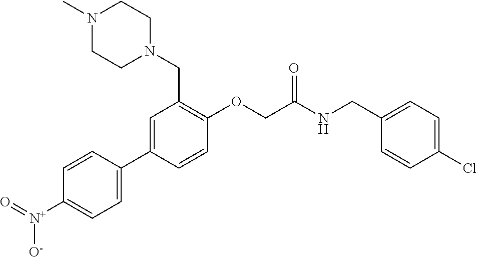 | NUCC-0201187 | 16 |
| 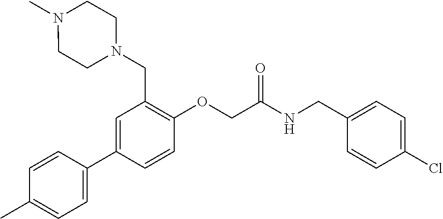 | NUCC-0201188 | 8.7 |
| 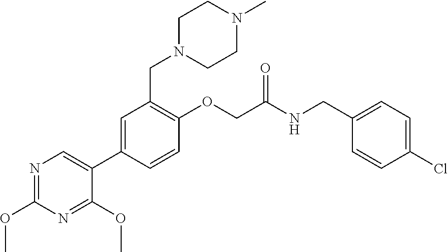 [SMILES: COc1ncc(-c2ccc(OCC(=O)NCc3ccc(Cl)cc3)c(CN3CCN(C)CC3)c2)c(OC)n1] | NUCC-0201189 | 92 |
| 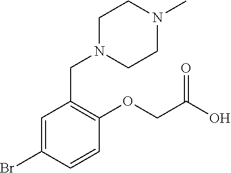 [SMILES: CN1CCN(Cc2cc(Br)ccc2OCC(=O)O)CC1] | NUCC-0201190 | NA |
| 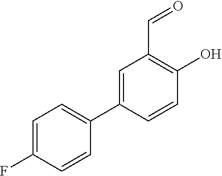 [SMILES: O=Cc1cc(-c2ccc(F)cc2)ccc1O] | NUCC-0201191 | 58 |
| 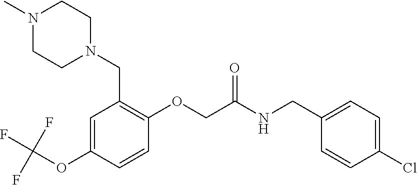 [SMILES: CN1CCN(Cc2cc(OC(F)(F)F)ccc2OCC(=O)NCc2ccc(Cl)cc2)CC1] | NUCC-0201228 | 160 |
| 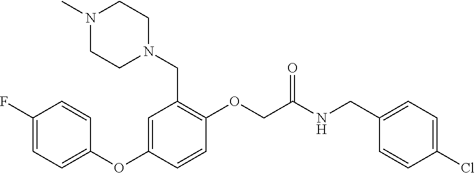 [SMILES: CN1CCN(Cc2cc(Oc3ccc(F)cc3)ccc2OCC(=O)NCc2ccc(Cl)cc2)CC1] | NUCC-0201229 | 7.1 |
| 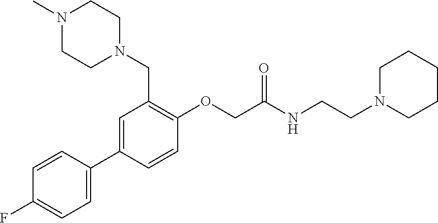 [SMILES: CN1CCN(Cc2cc(-c3ccc(F)cc3)ccc2OCC(=O)NCCN2CCCCC2)CC1] | NUCC-0201230 | >200 |
| 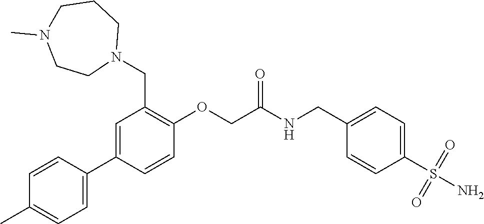 [SMILES: Cc1ccc(-c2ccc(OCC(=O)NCc3ccc(S(N)(=O)=O)cc3)c(CN3CCCN(C)CC3)c2)cc1] | NUCC-0201597 | >40 |
| 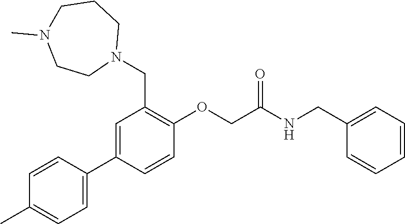 [SMILES: Cc1ccc(-c2ccc(OCC(=O)NCc3ccccc3)c(CN3CCCN(C)CC3)c2)cc1] | NUCC-0201598 | 20 |
| 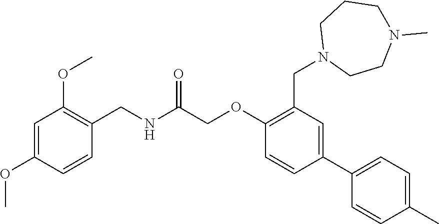 [SMILES: COc1ccc(CNC(=O)COc2ccc(-c3ccc(C)cc3)cc2CN2CCCN(C)CC2)c(OC)c1] | NUCC-0201599 | 15 |
| 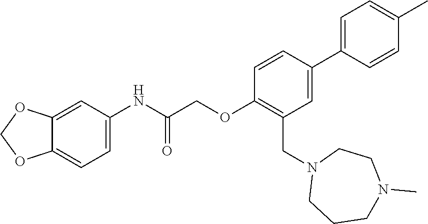 [SMILES: Cc1ccc(-c2ccc(OCC(=O)Nc3ccc4c(c3)OCO4)c(CN3CCCN(C)CC3)c2)cc1] | NUCC-0201600 | 9.2 |
| 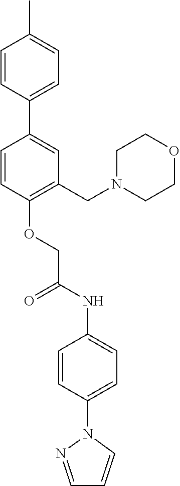 | NUCC-0201601 | 11 |
| 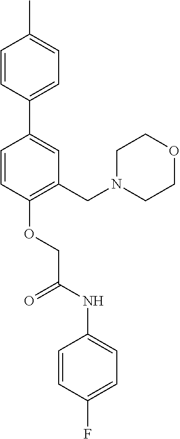 | NUCC-0201602 | 12 |
| 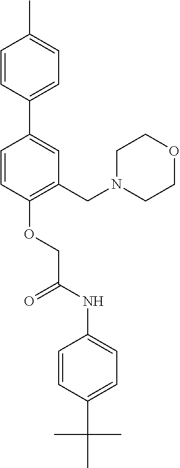 | NUCC-0201603 | NA |
| 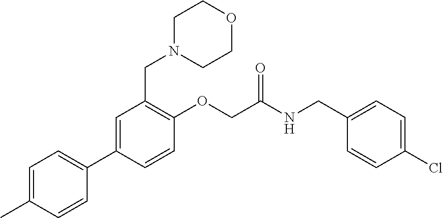 [SMILES: Cc1ccc(-c2ccc(OCC(=O)NCc3ccc(Cl)cc3)c(CN3CCOCC3)c2)cc1] | NUCC-0201604 | 8.1 |
| 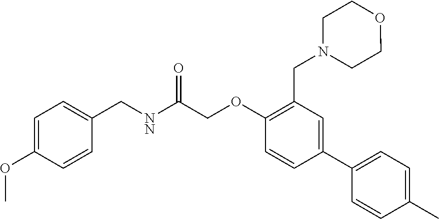 | NUCC-0201605 | 35 |
| 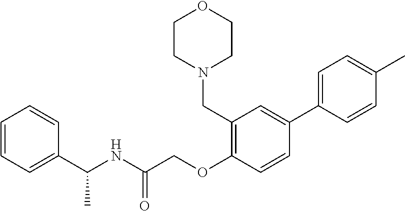 | NUCC-0201606 | 28 |
| 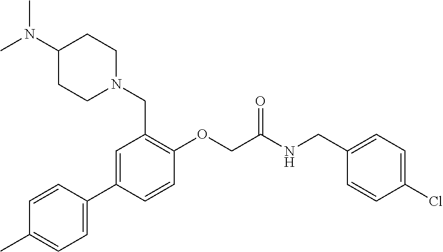 [SMILES: Cc1ccc(-c2ccc(OCC(=O)NCc3ccc(Cl)cc3)c(CN3CCC(N(C)C)CC3)c2)cc1] | NUCC-0201607 | 5.5 |
| 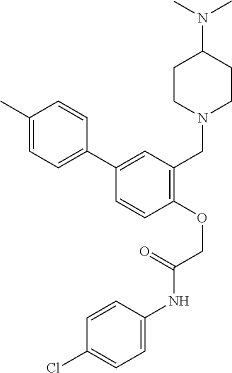 [SMILES: Cc1ccc(-c2ccc(OCC(=O)Nc3ccc(Cl)cc3)c(CN3CCC(N(C)C)CC3)c2)cc1] | NUCC-0201608 | 7 |
| 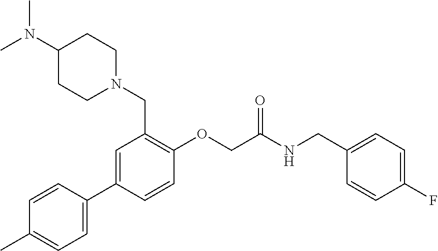 [SMILES: Cc1ccc(-c2ccc(OCC(=O)NCc3ccc(F)cc3)c(CN3CCC(N(C)C)CC3)c2)cc1] | NUCC-0201609 | 16 |
| 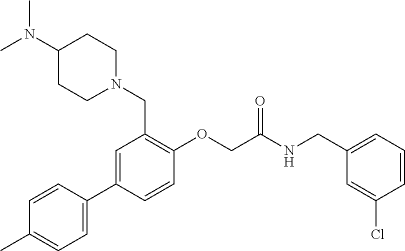 [SMILES: Cc1ccc(-c2ccc(OCC(=O)NCc3cccc(Cl)c3)c(CN3CCC(N(C)C)CC3)c2)cc1] | NUCC-0201610 | 5 |
| 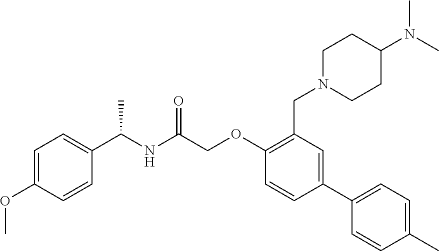 [SMILES: COc1ccc([C@H](C)NC(=O)COc2ccc(-c3ccc(C)cc3)cc2CN2CCC(N(C)C)CC2)cc1] | NUCC-0201611 | >40 |
| 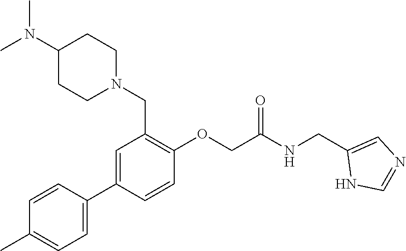 [SMILES: Cc1ccc(-c2ccc(OCC(=O)NCc3cnc[nH]3)c(CN3CCC(N(C)C)CC3)c2)cc1] | NUCC-0201612 | >40 |
| 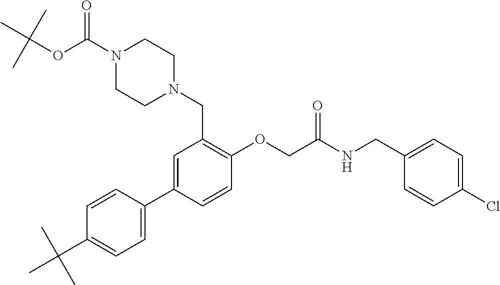 [SMILES: CC(C)(C)OC(=O)N1CCN(Cc2cc(-c3ccc(C(C)(C)C)cc3)ccc2OCC(=O)NCc2ccc(Cl)cc2)CC1] | NUCC-0201613 | NA |
| 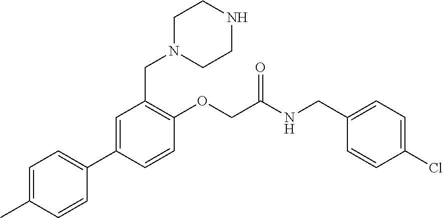 [SMILES: Cc1ccc(-c2ccc(OCC(=O)NCc3ccc(Cl)cc3)c(CN3CCNCC3)c2)cc1] | NUCC-0201614 | 7.2 |
| 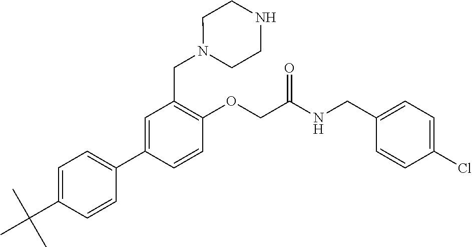 [SMILES: CC(C)(C)c1ccc(-c2ccc(OCC(=O)NCc3ccc(Cl)cc3)c(CN3CCNCC3)c2)cc1] | NUCC-0201615 | 6.3 |
| 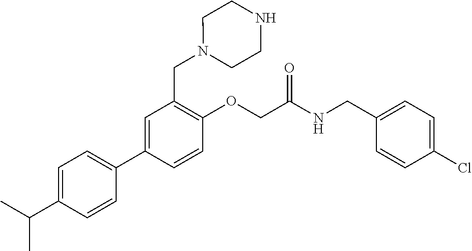 [SMILES: CC(C)c1ccc(-c2ccc(OCC(=O)NCc3ccc(Cl)cc3)c(CN3CCNCC3)c2)cc1] | NUCC-0201616 | 5.7 |
| 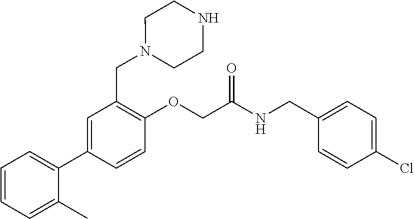 [SMILES: Cc1ccccc1-c1ccc(OCC(=O)NCc2ccc(Cl)cc2)c(CN2CCNCC2)c1] | NUCC-0201617 | 7.8 |
| 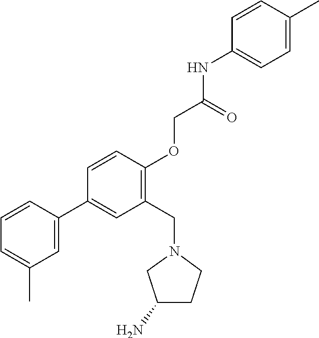 [SMILES: Cc1ccc(NC(=O)COc2ccc(-c3cccc(C)c3)cc2CN2CC[C@H](N)C2)cc1] | NUCC-0201618 | 8.8 |
| 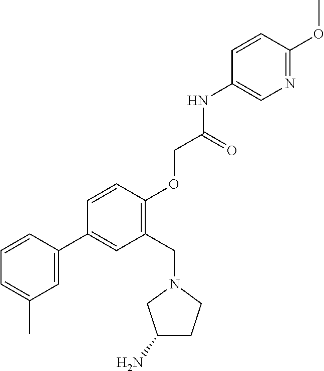 [SMILES: COc1ccc(NC(=O)COc2ccc(-c3cccc(C)c3)cc2CN2CC[C@H](N)C2)cn1] | NUCC-0201619 | 24 |
| 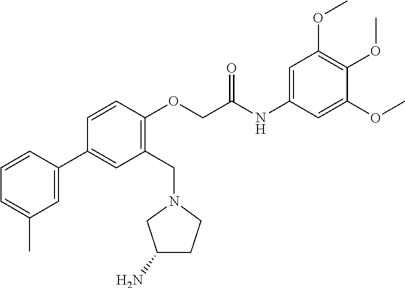 [SMILES: COc1cc(NC(=O)COc2ccc(-c3cccc(C)c3)cc2CN2CC[C@H](N)C2)cc(OC)c1OC] | NUCC-0201620 | 13 |
| 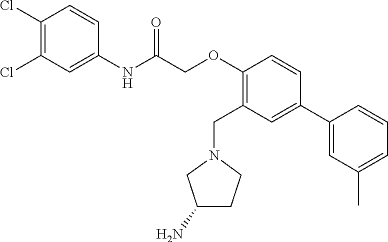 [SMILES: Cc1cccc(-c2ccc(OCC(=O)Nc3ccc(Cl)c(Cl)c3)c(CN3CC[C@H](N)C3)c2)c1] | NUCC-0201621 | 1.3 |
| 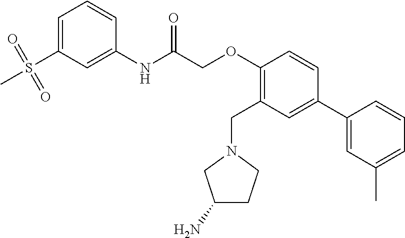 [SMILES: Cc1cccc(-c2ccc(OCC(=O)Nc3cccc(S(C)(=O)=O)c3)c(CN3CC[C@H](N)C3)c2)c1] | NUCC-0201622 | 19 |
| 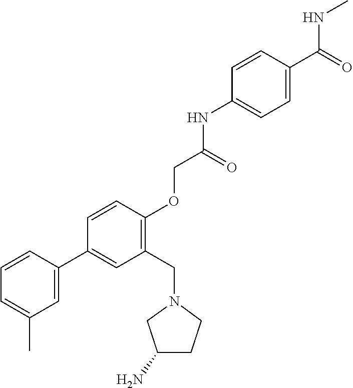 [SMILES: CNC(=O)c1ccc(NC(=O)COc2ccc(-c3cccc(C)c3)cc2CN2CC[C@H](N)C2)cc1] | NUCC-0201623 | 22 |
| 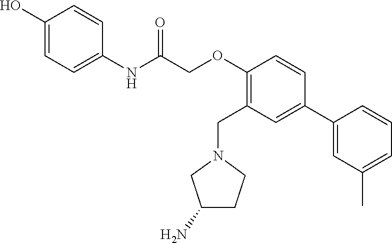 [SMILES: Cc1cccc(-c2ccc(OCC(=O)Nc3ccc(O)cc3)c(CN3CC[C@H](N)C3)c2)c1] | NUCC-0201624 | 20 |
| 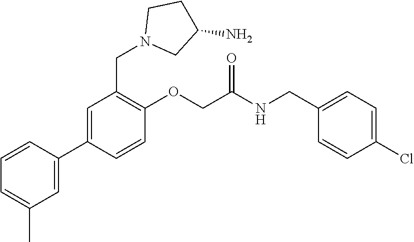 [SMILES: Cc1cccc(-c2ccc(OCC(=O)NCc3ccc(Cl)cc3)c(CN3CC[C@H](N)C3)c2)c1] | NUCC-0201625 | 6.1 |
| 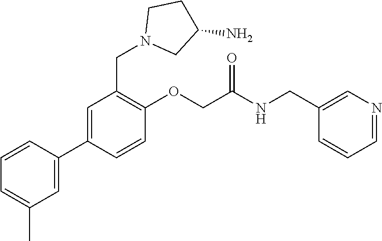 [SMILES: Cc1cccc(-c2ccc(OCC(=O)NCc3cccnc3)c(CN3CC[C@H](N)C3)c2)c1] | NUCC-0201626 | >40 |
| 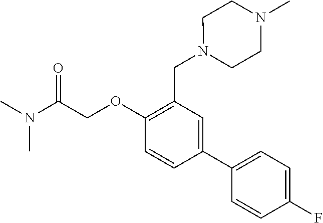 [SMILES: CN1CCN(Cc2cc(-c3ccc(F)cc3)ccc2OCC(=O)N(C)C)CC1] | NUCC-0201627 | NA |
| 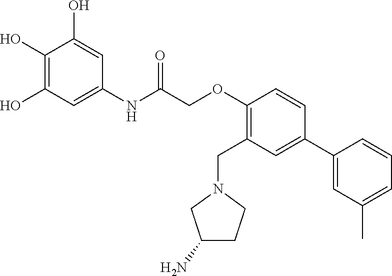 [SMILES: Cc1cccc(-c2ccc(OCC(=O)Nc3cc(O)c(O)c(O)c3)c(CN3CC[C@H](N)C3)c2)c1] | NUCC-0201628 | 7.8 |
| 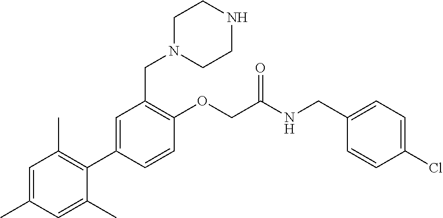 [SMILES: Cc1cc(C)c(-c2ccc(OCC(=O)NCc3ccc(Cl)cc3)c(CN3CCNCC3)c2)c(C)c1] | NUCC-0201629 | 3.1 |
| 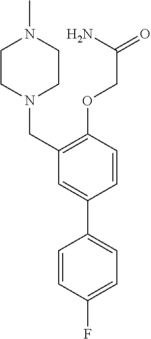 [SMILES: CN1CCN(Cc2cc(-c3ccc(F)cc3)ccc2OCC(N)=O)CC1] | NUCC-0201707 | NA |
| 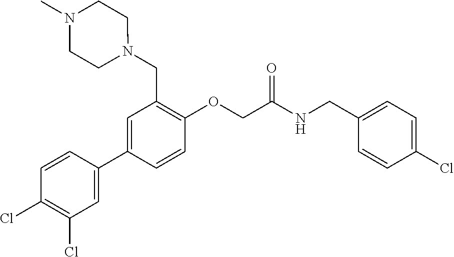 [SMILES: CN1CCN(Cc2cc(-c3ccc(Cl)c(Cl)c3)ccc2OCC(=O)NCc2ccc(Cl)cc2)CC1] | NUCC-0201708 | <0.74 |
| 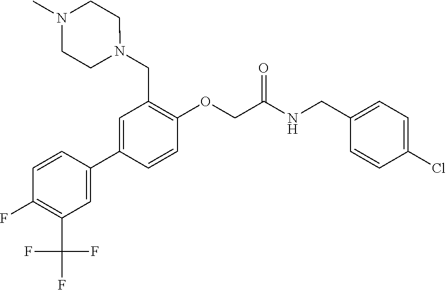 [SMILES: CN1CCN(Cc2cc(-c3ccc(F)c(C(F)(F)F)c3)ccc2OCC(=O)NCc2ccc(Cl)cc2)CC1] | NUCC-0201709 | 2.8 |
| 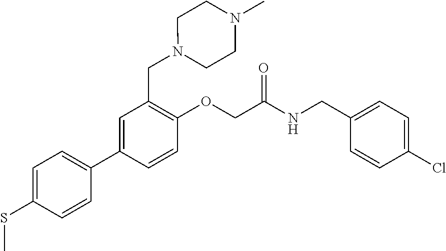 [SMILES: CSc1ccc(-c2ccc(OCC(=O)NCc3ccc(Cl)cc3)c(CN3CCN(C)CC3)c2)cc1] | NUCC-0201710 | 2.9 |
| 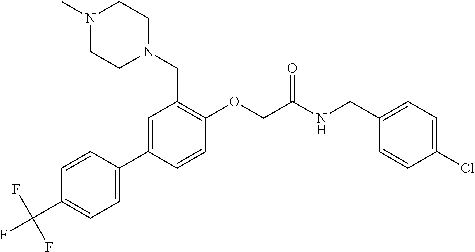 [SMILES: CN1CCN(Cc2cc(-c3ccc(C(F)(F)F)cc3)ccc2OCC(=O)NCc2ccc(Cl)cc2)CC1] | NUCC-0201711 | 3.9 |
| 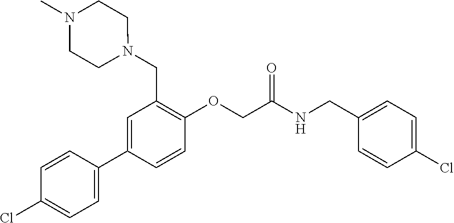 [SMILES: CN1CCN(Cc2cc(-c3ccc(Cl)cc3)ccc2OCC(=O)NCc2ccc(Cl)cc2)CC1] | NUCC-0201712 | 3 |
| 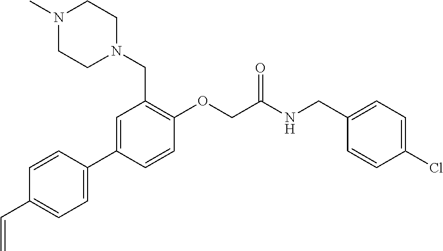 [SMILES: C=Cc1ccc(-c2ccc(OCC(=O)NCc3ccc(Cl)cc3)c(CN3CCN(C)CC3)c2)cc1] | NUCC-0201713 | 4.7 |
| 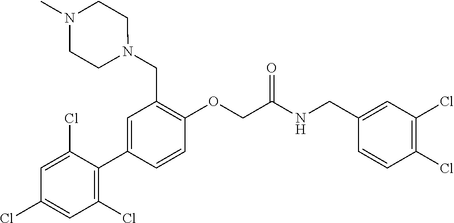 [SMILES: CN1CCN(Cc2cc(-c3c(Cl)cc(Cl)cc3Cl)ccc2OCC(=O)NCc2ccc(Cl)c(Cl)c2)CC1] | NUCC-0201969 | 1.1 |
| 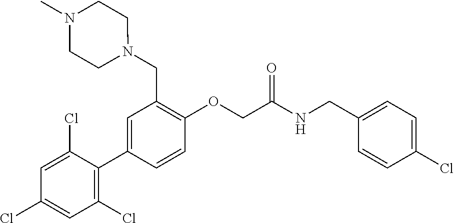 [SMILES: CN1CCN(Cc2cc(-c3c(Cl)cc(Cl)cc3Cl)ccc2OCC(=O)NCc2ccc(Cl)cc2)CC1] | NUCC-0201970 | <0.74 |
| 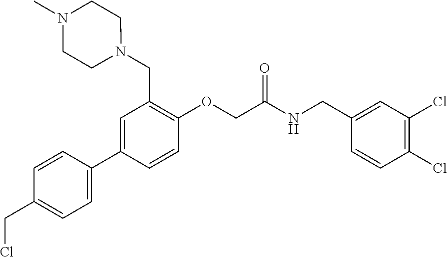 [SMILES: CN1CCN(Cc2cc(-c3ccc(CCl)cc3)ccc2OCC(=O)NCc2ccc(Cl)c(Cl)c2)CC1] | NUCC-0201971 | 6.6 |
| 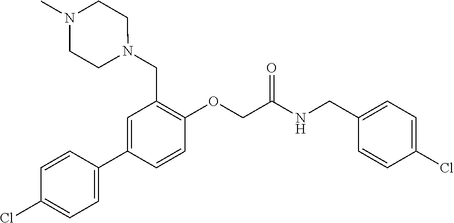 [SMILES: CN1CCN(Cc2cc(-c3ccc(Cl)cc3)ccc2OCC(=O)NCc2ccc(Cl)cc2)CC1] | NUCC-0201972 | 5 |
| 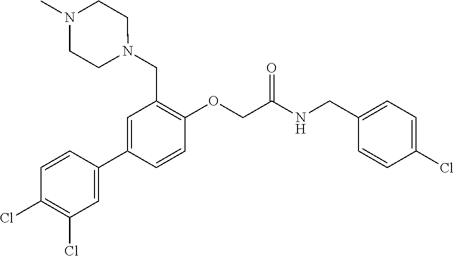 [SMILES: CN1CCN(Cc2cc(-c3ccc(Cl)c(Cl)c3)ccc2OCC(=O)NCc2ccc(Cl)cc2)CC1] | NUCC-0201973 | 1.3 |
| 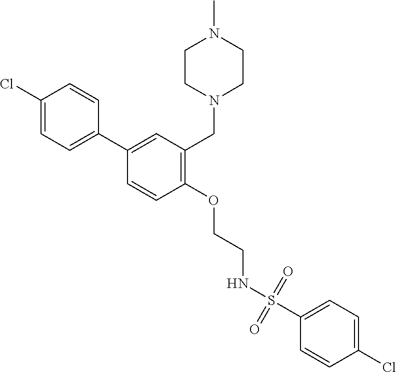 [SMILES: CN1CCN(Cc2cc(-c3ccc(Cl)cc3)ccc2OCCNS(=O)(=O)c2ccc(Cl)cc2)CC1] | NUCC-0201974 | 1.8 |
| 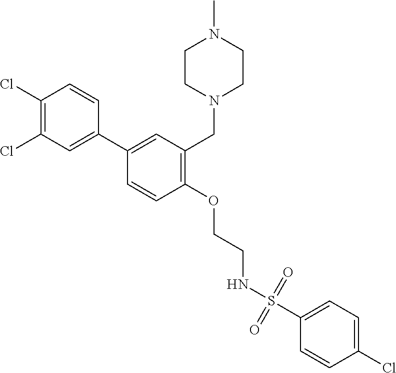 [SMILES: CN1CCN(Cc2cc(-c3ccc(Cl)c(Cl)c3)ccc2OCCNS(=O)(=O)c2ccc(Cl)cc2)CC1] | NUCC-0201975 | 1.7 |
| 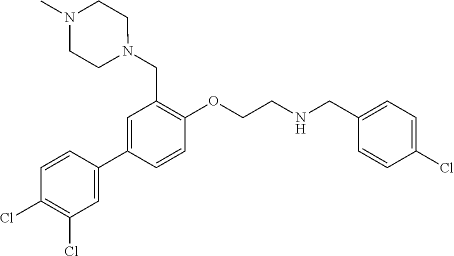 [SMILES: CN1CCN(Cc2cc(-c3ccc(Cl)c(Cl)c3)ccc2OCCNCc2ccc(Cl)cc2)CC1] | NUCC-0201976 | 0.83 |
| 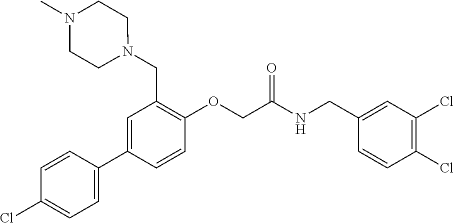 [SMILES: CN1CCN(Cc2cc(-c3ccc(Cl)cc3)ccc2OCC(=O)NCc2ccc(Cl)c(Cl)c2)CC1] | NUCC-0201977 | 0.87 |
| 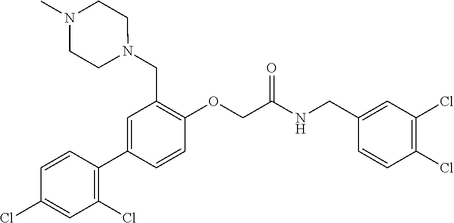 [SMILES: CN1CCN(Cc2cc(-c3ccc(Cl)cc3Cl)ccc2OCC(=O)NCc2ccc(Cl)c(Cl)c2)CC1] | NUCC-0201978 | <0.74 |
| 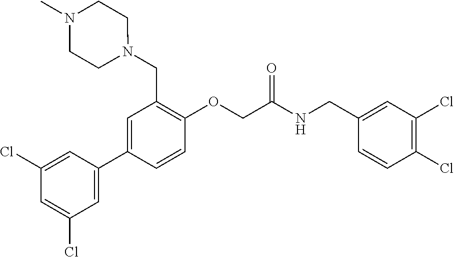 [SMILES: CN1CCN(Cc2cc(-c3cc(Cl)cc(Cl)c3)ccc2OCC(=O)NCc2ccc(Cl)c(Cl)c2)CC1] | NUCC-0201979 | <0.74 |
| 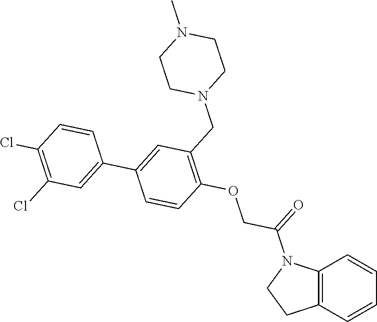 [SMILES: CN1CCN(Cc2cc(-c3ccc(Cl)c(Cl)c3)ccc2OCC(=O)N2CCc3ccccc32)CC1] | NUCC-0201980 | 1.2 |
| 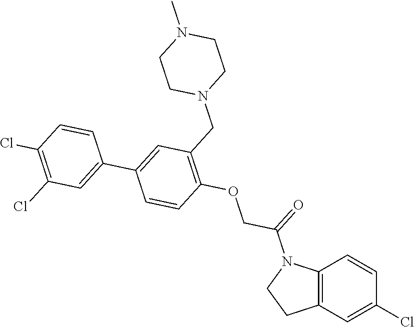 [SMILES: CN1CCN(Cc2cc(-c3ccc(Cl)c(Cl)c3)ccc2OCC(=O)N2CCc3cc(Cl)ccc32)CC1] | NUCC-0201981 | <0.74 |
| 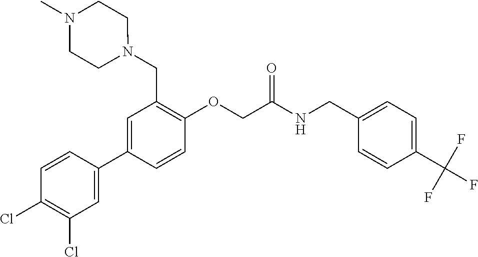 [SMILES: CN1CCN(Cc2cc(-c3ccc(Cl)c(Cl)c3)ccc2OCC(=O)NCc2ccc(C(F)(F)F)cc2)CC1] | NUCC-0201982 | 2.5 |
| 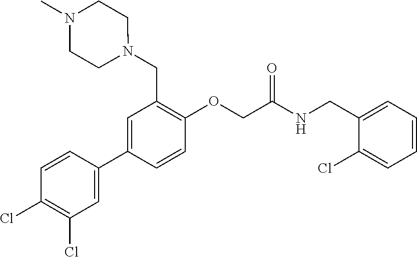 [SMILES: CN1CCN(Cc2cc(-c3ccc(Cl)c(Cl)c3)ccc2OCC(=O)NCc2ccccc2Cl)CC1] | NUCC-0201983 | 0.91 |
| 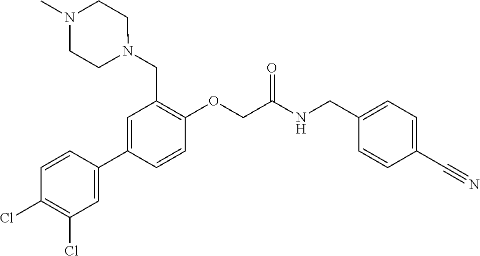 [SMILES: CN1CCN(Cc2cc(-c3ccc(Cl)c(Cl)c3)ccc2OCC(=O)NCc2ccc(C#N)cc2)CC1] | NUCC-0201984 | 5.3 |
| 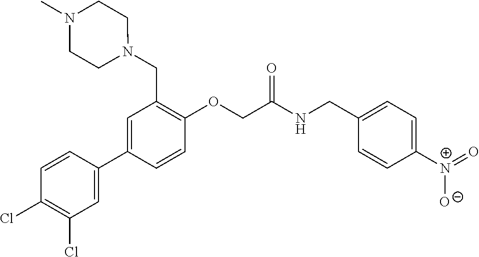 [SMILES: CN1CCN(Cc2cc(-c3ccc(Cl)c(Cl)c3)ccc2OCC(=O)NCc2ccc([N+](=O)[O-])cc2)CC1] | NUCC-0201985 | 2.5 |
| 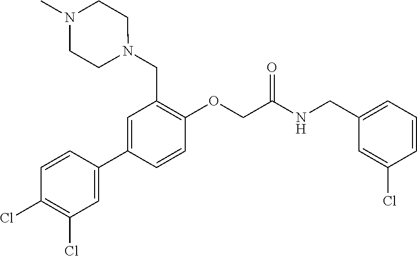 [SMILES: CN1CCN(Cc2cc(-c3ccc(Cl)c(Cl)c3)ccc2OCC(=O)NCc2cccc(Cl)c2)CC1] | NUCC-0201986 | 1.1 |
| 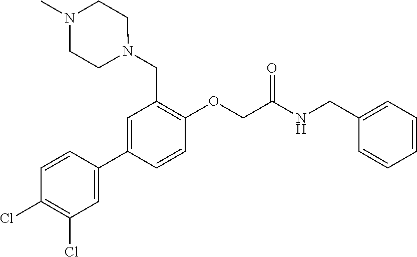 [SMILES: CN1CCN(Cc2cc(-c3ccc(Cl)c(Cl)c3)ccc2OCC(=O)NCc2ccccc2)CC1] | NUCC-0201987 | 2.5 |
| 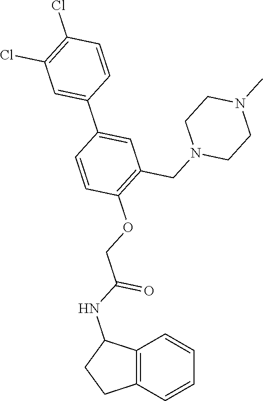 [SMILES: CN1CCN(Cc2cc(-c3ccc(Cl)c(Cl)c3)ccc2OCC(=O)NC2CCc3ccccc32)CC1] | NUCC-0201988 | 1.4 |
| 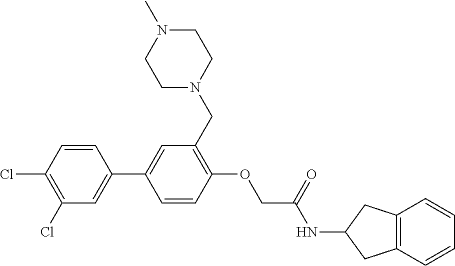 [SMILES: CN1CCN(Cc2cc(-c3ccc(Cl)c(Cl)c3)ccc2OCC(=O)NC2Cc3ccccc3C2)CC1] | NUCC-0201989 | 1.3 |
| 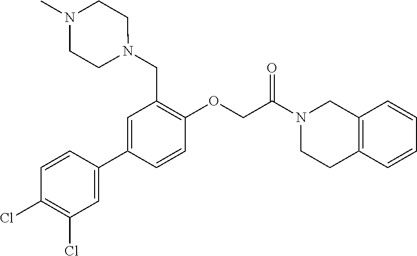 [SMILES: CN1CCN(Cc2cc(-c3ccc(Cl)c(Cl)c3)ccc2OCC(=O)N2CCc3ccccc3C2)CC1] | NUCC-0201990 | <0.74 |
| 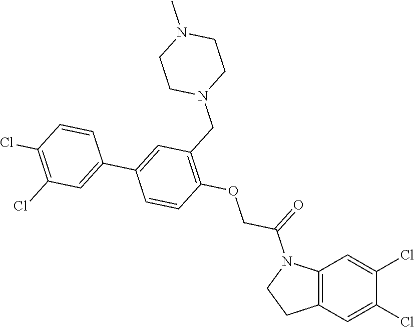 [SMILES: CN1CCN(Cc2cc(-c3ccc(Cl)c(Cl)c3)ccc2OCC(=O)N2CCc3cc(Cl)c(Cl)cc32)CC1] | NUCC-0201991 | <0.74 |
| 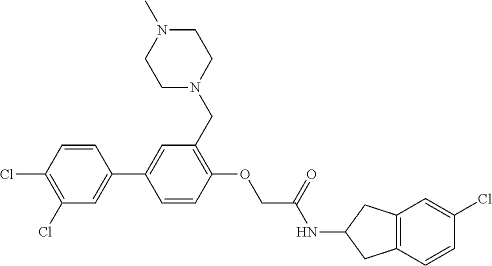 [SMILES: CN1CCN(Cc2cc(-c3ccc(Cl)c(Cl)c3)ccc2OCC(=O)NC2Cc3ccc(Cl)cc3C2)CC1] | NUCC-0201992 | <0.74 |
| 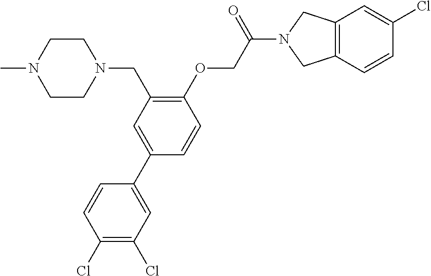 [SMILES: CN1CCN(Cc2cc(-c3ccc(Cl)c(Cl)c3)ccc2OCC(=O)N2Cc3ccc(Cl)cc3C2)CC1] | NUCC-0201993 | 1.9 |
| 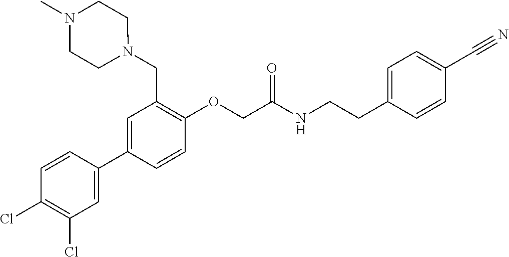 [SMILES: CN1CCN(Cc2cc(-c3ccc(Cl)c(Cl)c3)ccc2OCC(=O)NCCc2ccc(C#N)cc2)CC1] | NUCC-0201994 | 3.4 |
| 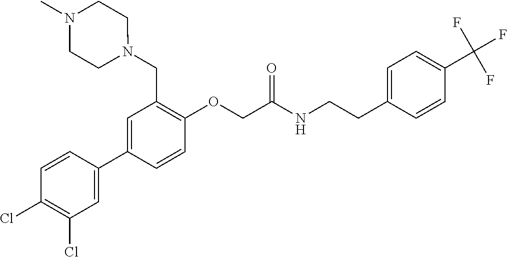 [SMILES: CN1CCN(Cc2cc(-c3ccc(Cl)c(Cl)c3)ccc2OCC(=O)NCCc2ccc(C(F)(F)F)cc2)CC1] | NUCC-0201995 | <0.74 |
| 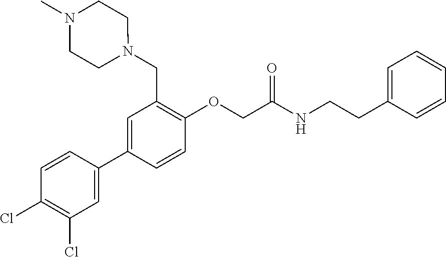 [SMILES: CN1CCN(Cc2cc(-c3ccc(Cl)c(Cl)c3)ccc2OCC(=O)NCCc2ccccc2)CC1] | NUCC-0201996 | 1.1 |
| 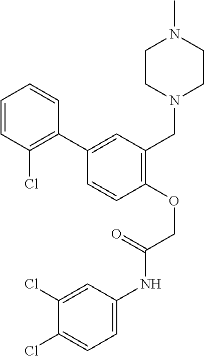 [SMILES: CN1CCN(Cc2cc(-c3ccccc3Cl)ccc2OCC(=O)Nc2ccc(Cl)c(Cl)c2)CC1] | NUCC-0201997 | <0.74 |
| 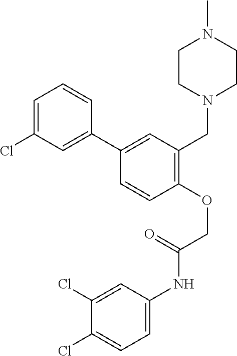 [SMILES: CN1CCN(Cc2cc(-c3cccc(Cl)c3)ccc2OCC(=O)Nc2ccc(Cl)c(Cl)c2)CC1] | NUCC-0201998 | <0.74 |
| 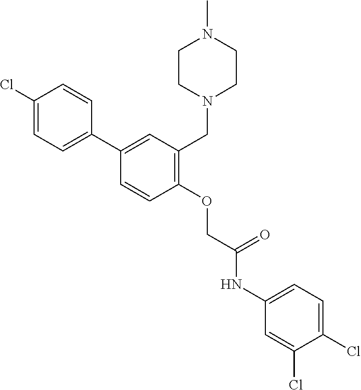 [SMILES: CN1CCN(Cc2cc(-c3ccc(Cl)cc3)ccc2OCC(=O)Nc2ccc(Cl)c(Cl)c2)CC1] | NUCC-0201999 | <0.74 |
| 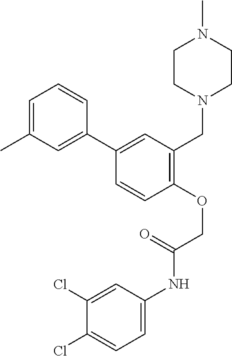 [SMILES: Cc1cccc(-c2ccc(OCC(=O)Nc3ccc(Cl)c(Cl)c3)c(CN3CCN(C)CC3)c2)c1] | NUCC-0202000 | 0.76 |
| 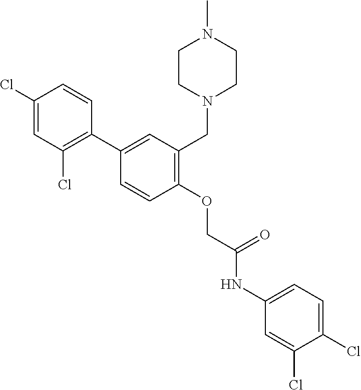 [SMILES: CN1CCN(Cc2cc(-c3ccc(Cl)cc3Cl)ccc2OCC(=O)Nc2ccc(Cl)c(Cl)c2)CC1] | NUCC-0202001 | <0.74 |
| 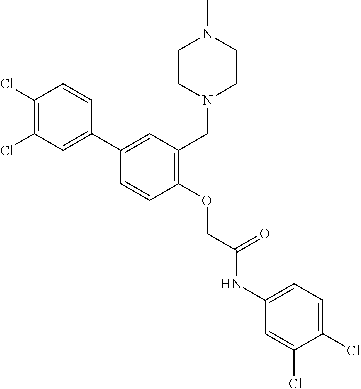 [SMILES: CN1CCN(Cc2cc(-c3ccc(Cl)c(Cl)c3)ccc2OCC(=O)Nc2ccc(Cl)c(Cl)c2)CC1] | NUCC-0202002 | <0.74 |
| 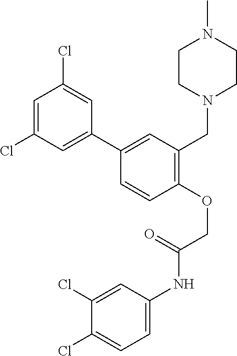 [SMILES: CN1CCN(Cc2cc(-c3cc(Cl)cc(Cl)c3)ccc2OCC(=O)Nc2ccc(Cl)c(Cl)c2)CC1] | NUCC-0202003 | <0.74 |
| 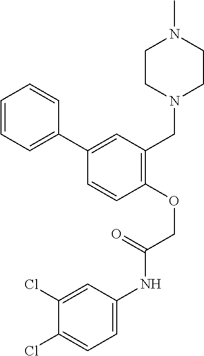 [SMILES: CN1CCN(Cc2cc(-c3ccccc3)ccc2OCC(=O)Nc2ccc(Cl)c(Cl)c2)CC1] | NUCC-0202004 | 0.89 |
| 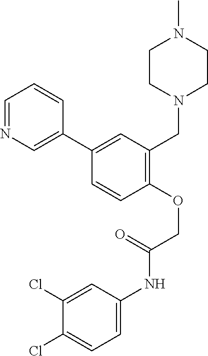 [SMILES: CN1CCN(Cc2cc(-c3cccnc3)ccc2OCC(=O)Nc2ccc(Cl)c(Cl)c2)CC1] | NUCC-0202005 | 5.5 |
| 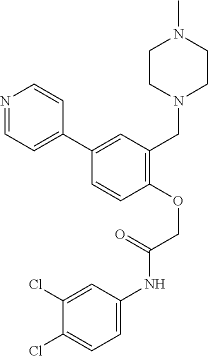 [SMILES: CN1CCN(Cc2cc(-c3ccncc3)ccc2OCC(=O)Nc2ccc(Cl)c(Cl)c2)CC1] | NUCC-0202006 | 5.3 |
| 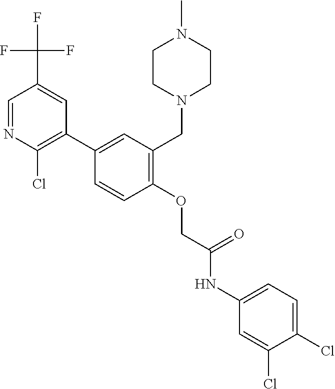 [SMILES: CN1CCN(Cc2cc(-c3cc(C(F)(F)F)cnc3Cl)ccc2OCC(=O)Nc2ccc(Cl)c(Cl)c2)CC1] | NUCC-0202007 | 4.6 |
| 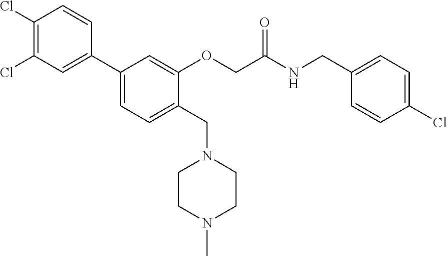 [SMILES: CN1CCN(Cc2ccc(-c3ccc(Cl)c(Cl)c3)cc2OCC(=O)NCc2ccc(Cl)cc2)CC1] | NUCC-0202008 | 1.2 |
| 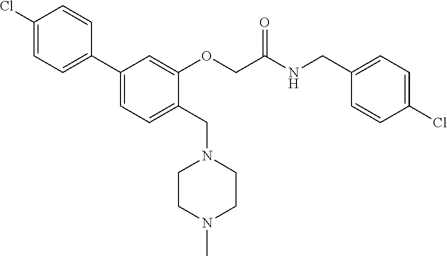 [SMILES: CN1CCN(Cc2ccc(-c3ccc(Cl)cc3)cc2OCC(=O)NCc2ccc(Cl)cc2)CC1] | NUCC-0202009 | 1 |
| 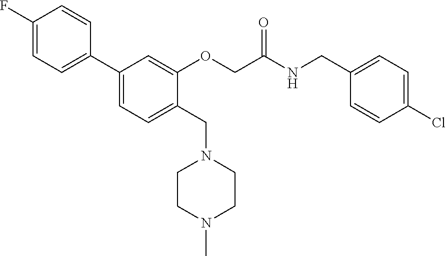 [SMILES: CN1CCN(Cc2ccc(-c3ccc(F)cc3)cc2OCC(=O)NCc2ccc(Cl)cc2)CC1] | NUCC-0202010 | 5.7 |
| 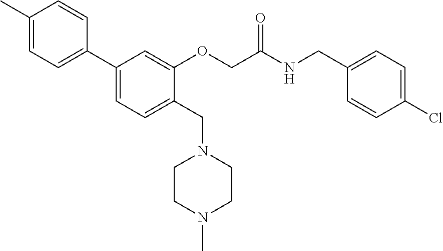 [SMILES: Cc1ccc(-c2ccc(CN3CCN(C)CC3)c(OCC(=O)NCc3ccc(Cl)cc3)c2)cc1] | NUCC-0202011 | 1.5 |
| 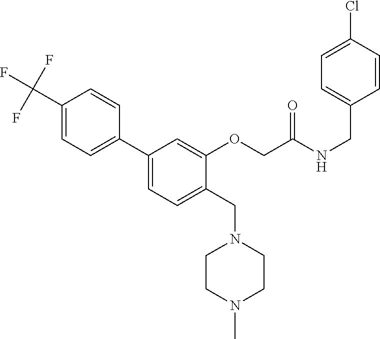 [SMILES: CN1CCN(Cc2ccc(-c3ccc(C(F)(F)F)cc3)cc2OCC(=O)NCc2ccc(Cl)cc2)CC1] | NUCC-0202012 | 2.1 |
| 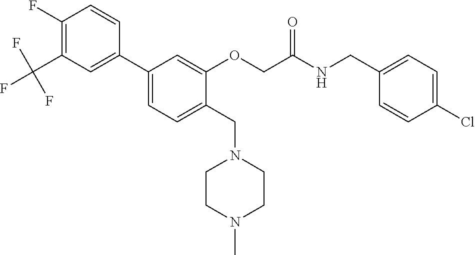 [SMILES: CN1CCN(Cc2ccc(-c3ccc(F)c(C(F)(F)F)c3)cc2OCC(=O)NCc2ccc(Cl)cc2)CC1] | NUCC-0202013 | 1.4 |
| 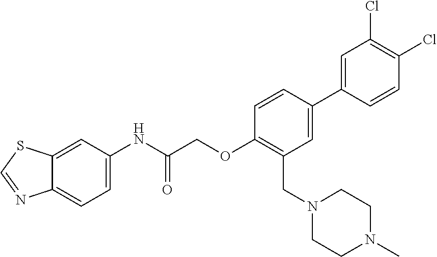 [SMILES: CN1CCN(Cc2cc(-c3ccc(Cl)c(Cl)c3)ccc2OCC(=O)Nc2ccc3ncsc3c2)CC1] | NUCC-0202014 | 0.81 |
| 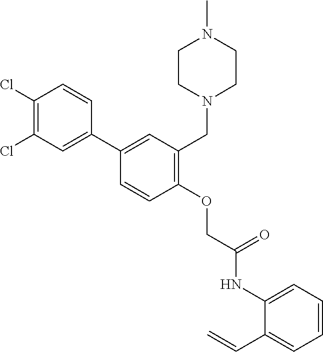 [SMILES: C=Cc1ccccc1NC(=O)COc1ccc(-c2ccc(Cl)c(Cl)c2)cc1CN1CCN(C)CC1] | NUCC-0202015 | 1.1 |
| 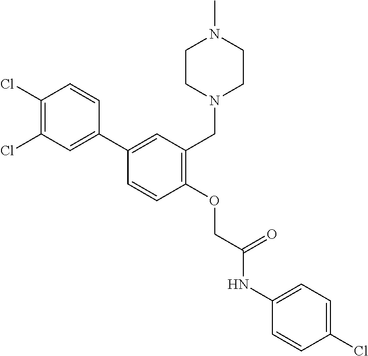 [SMILES: CN1CCN(Cc2cc(-c3ccc(Cl)c(Cl)c3)ccc2OCC(=O)Nc2ccc(Cl)cc2)CC1] | NUCC-0202016 | 0.78 |
| 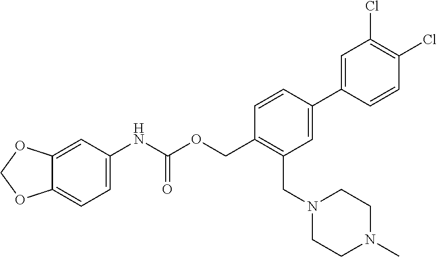 [SMILES: CN1CCN(Cc2cc(-c3ccc(Cl)c(Cl)c3)ccc2COC(=O)Nc2ccc3c(c2)OCO3)CC1] | NUCC-0202017 | <0.74 |
| 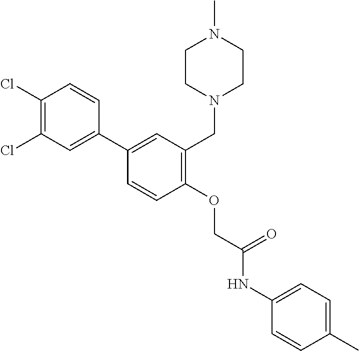 [SMILES: Cc1ccc(NC(=O)COc2ccc(-c3ccc(Cl)c(Cl)c3)cc2CN2CCN(C)CC2)cc1] | NUCC-0202018 | 1 |
| 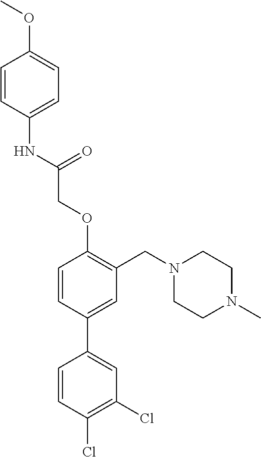 [SMILES: COc1ccc(NC(=O)COc2ccc(-c3ccc(Cl)c(Cl)c3)cc2CN2CCN(C)CC2)cc1] | NUCC-0202019 | 1.3 |
| 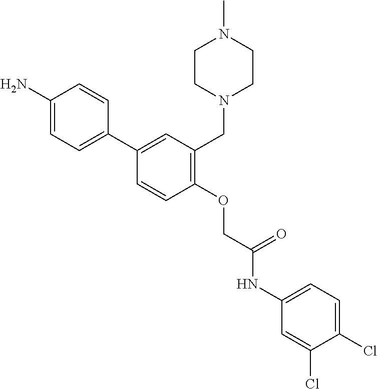 [SMILES: CN1CCN(Cc2cc(-c3ccc(N)cc3)ccc2OCC(=O)Nc2ccc(Cl)c(Cl)c2)CC1] | NUCC-0202020 | 1.5 |
| 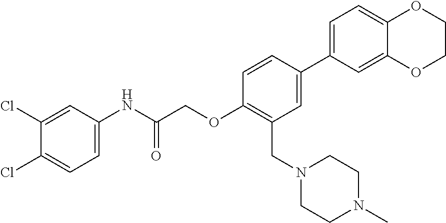 [SMILES: CN1CCN(Cc2cc(-c3ccc4c(c3)OCCO4)ccc2OCC(=O)Nc2ccc(Cl)c(Cl)c2)CC1] | NUCC-0202021 | 1.4 |
| 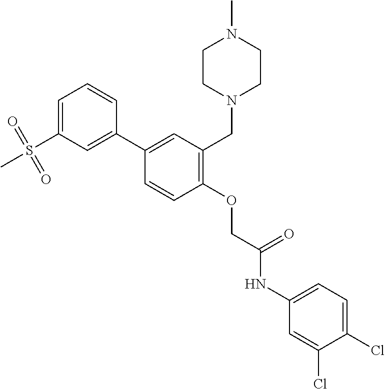 [SMILES: CN1CCN(Cc2cc(-c3cccc(S(C)(=O)=O)c3)ccc2OCC(=O)Nc2ccc(Cl)c(Cl)c2)CC1] | NUCC-0202022 | 2.7 |
| 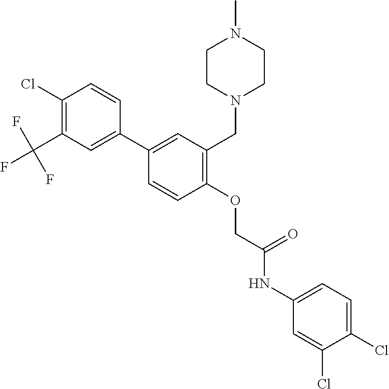 [SMILES: CN1CCN(Cc2cc(-c3ccc(Cl)c(C(F)(F)F)c3)ccc2OCC(=O)Nc2ccc(Cl)c(Cl)c2)CC1] | NUCC-0202023 | 1.3 |
| 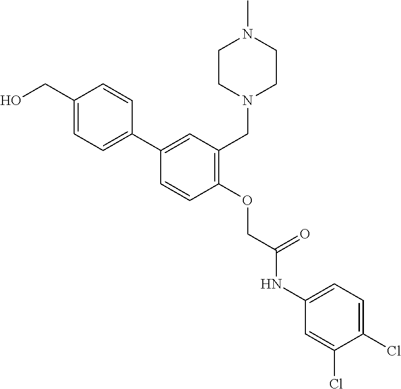 [SMILES: CN1CCN(Cc2cc(-c3ccc(CO)cc3)ccc2OCC(=O)Nc2ccc(Cl)c(Cl)c2)CC1] | NUCC-0202024 | 3.2 |
| 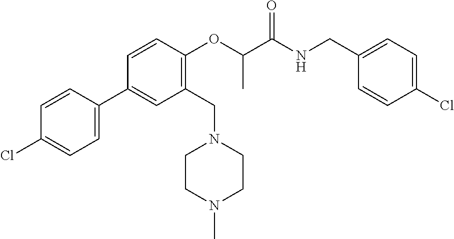 [SMILES: CC(Oc1ccc(-c2ccc(Cl)cc2)cc1CN1CCN(C)CC1)C(=O)NCc1ccc(Cl)cc1] | NUCC-0202025 | 1.5 |
| 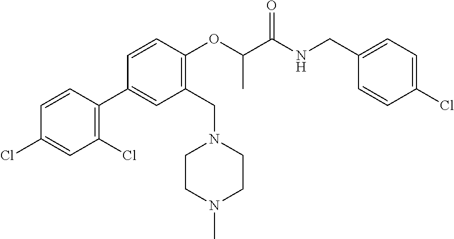 [SMILES: CC(Oc1ccc(-c2ccc(Cl)cc2Cl)cc1CN1CCN(C)CC1)C(=O)NCc1ccc(Cl)cc1] | NUCC-0202026 | 0.91 |
| 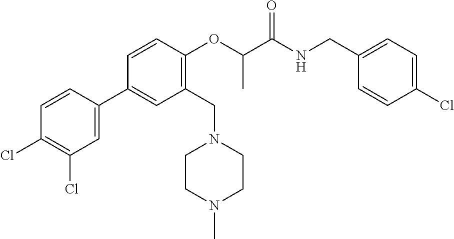 [SMILES: CC(Oc1ccc(-c2ccc(Cl)c(Cl)c2)cc1CN1CCN(C)CC1)C(=O)NCc1ccc(Cl)cc1] | NUCC-0202027 | 0.94 |
| 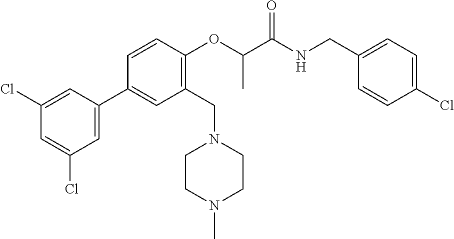 [SMILES: CC(Oc1ccc(-c2cc(Cl)cc(Cl)c2)cc1CN1CCN(C)CC1)C(=O)NCc1ccc(Cl)cc1] | NUCC-0202028 | 0.86 |
| 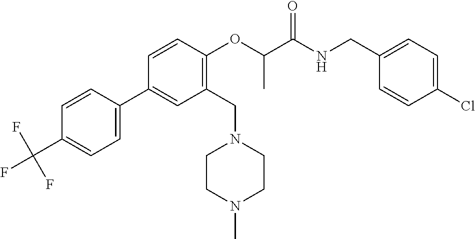 [SMILES: CC(Oc1ccc(-c2ccc(C(F)(F)F)cc2)cc1CN1CCN(C)CC1)C(=O)NCc1ccc(Cl)cc1] | NUCC-0202029 | 4.5 |
| 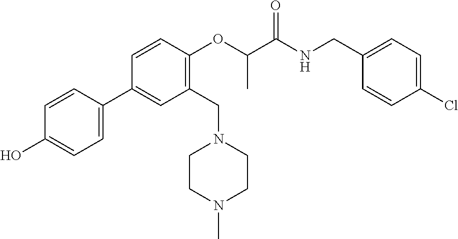 [SMILES: CC(Oc1ccc(-c2ccc(O)cc2)cc1CN1CCN(C)CC1)C(=O)NCc1ccc(Cl)cc1] | NUCC-0202030 | 14 |
| 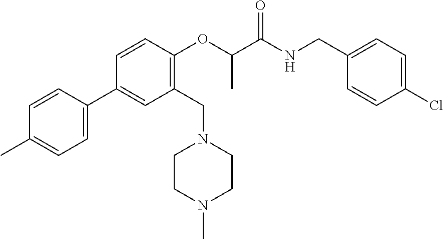 [SMILES: Cc1ccc(-c2ccc(OC(C)C(=O)NCc3ccc(Cl)cc3)c(CN3CCN(C)CC3)c2)cc1] | NUCC-0202031 | 3.4 |
| 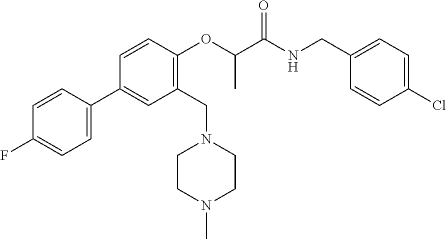 [SMILES: CC(Oc1ccc(-c2ccc(F)cc2)cc1CN1CCN(C)CC1)C(=O)NCc1ccc(Cl)cc1] | NUCC-0202032 | 3.2 |
| 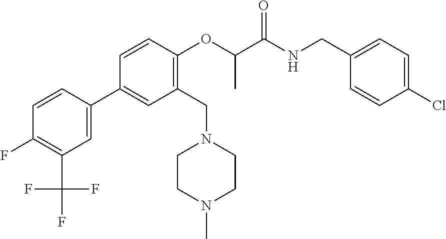 [SMILES: CC(Oc1ccc(-c2ccc(F)c(C(F)(F)F)c2)cc1CN1CCN(C)CC1)C(=O)NCc1ccc(Cl)cc1] | NUCC-0202033 | 0.99 |
| 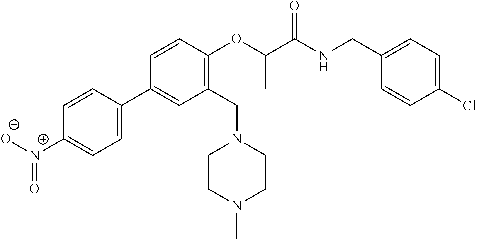 [SMILES: CC(Oc1ccc(-c2ccc([N+](=O)[O-])cc2)cc1CN1CCN(C)CC1)C(=O)NCc1ccc(Cl)cc1] | NUCC-0202034 | 2.7 |
| 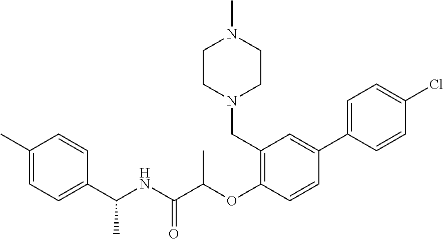 [SMILES: Cc1ccc([C@@H](C)NC(=O)C(C)Oc2ccc(-c3ccc(Cl)cc3)cc2CN2CCN(C)CC2)cc1] | NUCC-0202046 | 0.84 |
| 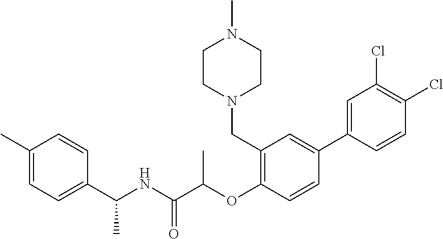 [SMILES: Cc1ccc([C@@H](C)NC(=O)C(C)Oc2ccc(-c3ccc(Cl)c(Cl)c3)cc2CN2CCN(C)CC2)cc1] | NUCC-0202047 | <0.74 |
| 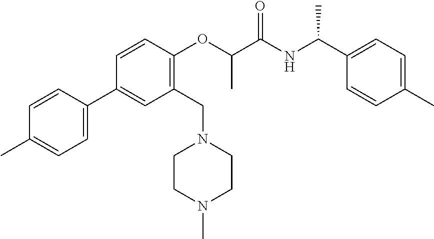 | NUCC-0202048 | 1.9 |
| 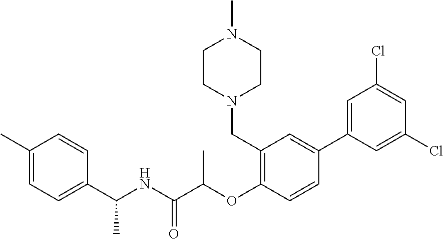 [SMILES: Cc1ccc([C@@H](C)NC(=O)C(C)Oc2ccc(-c3cc(Cl)cc(Cl)c3)cc2CN2CCN(C)CC2)cc1] | NUCC-0202049 | <0.74 |
| 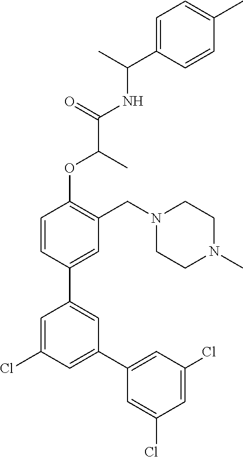 [SMILES: Cc1ccc(C(C)NC(=O)C(C)Oc2ccc(-c3cc(Cl)cc(-c4cc(Cl)cc(Cl)c4)c3)cc2CN2CCN(C)CC2)cc1] | NUCC-0202050 | <0.74 |
| 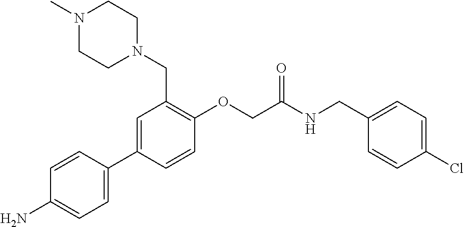 [SMILES: CN1CCN(Cc2cc(-c3ccc(N)cc3)ccc2OCC(=O)NCc2ccc(Cl)cc2)CC1] | NUCC-0202053 | 18 |
| 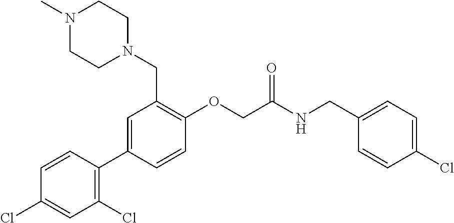 [SMILES: CN1CCN(Cc2cc(-c3ccc(Cl)cc3Cl)ccc2OCC(=O)NCc2ccc(Cl)cc2)CC1] | NUCC-0202054 | <0.74 |
| 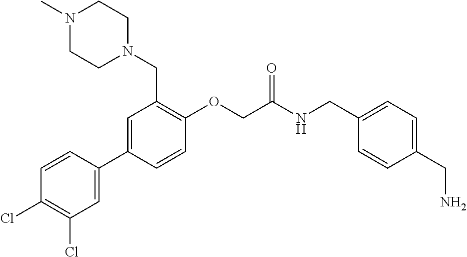 [SMILES: CN1CCN(Cc2cc(-c3ccc(Cl)c(Cl)c3)ccc2OCC(=O)NCc2ccc(CN)cc2)CC1] | NUCC-0202219 | NA |
| 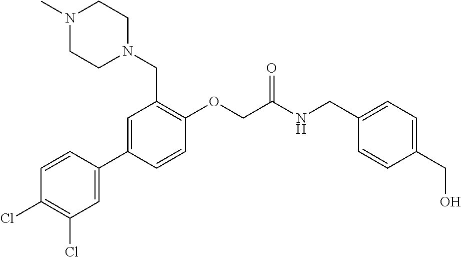 [SMILES: CN1CCN(Cc2cc(-c3ccc(Cl)c(Cl)c3)ccc2OCC(=O)NCc2ccc(CO)cc2)CC1] | NUCC-0202220 | NA |
| 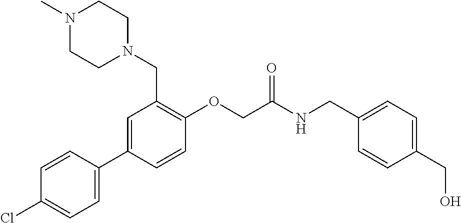 [SMILES: CN1CCN(Cc2cc(-c3ccc(Cl)cc3)ccc2OCC(=O)NCc2ccc(CO)cc2)CC1] | NUCC-0202221 | NA |
| 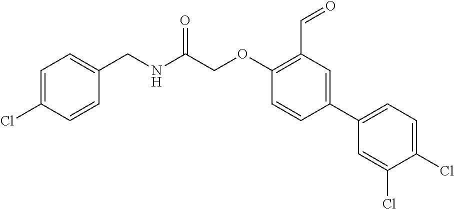 [SMILES: O=Cc1cc(-c2ccc(Cl)c(Cl)c2)ccc1OCC(=O)NCc1ccc(Cl)cc1] | NUCC-0202222 | NA |
| 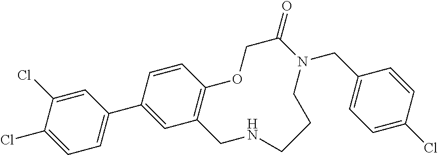 [SMILES: O=C1COc2ccc(-c3ccc(Cl)c(Cl)c3)cc2CNCCCN1Cc1ccc(Cl)cc1] | NUCC-0202223 | NA |
| 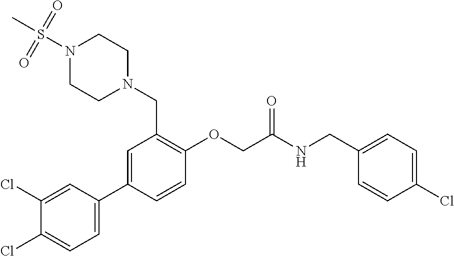 [SMILES: CS(=O)(=O)N1CCN(Cc2cc(-c3ccc(Cl)c(Cl)c3)ccc2OCC(=O)NCc2ccc(Cl)cc2)CC1] | NUCC-0202235 | NA |
| 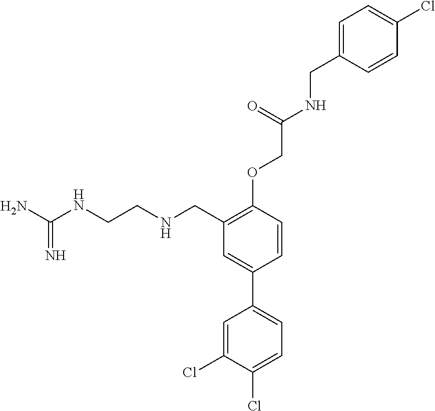 [SMILES: N=C(N)NCCNCc1cc(-c2ccc(Cl)c(Cl)c2)ccc1OCC(=O)NCc1ccc(Cl)cc1] | NUCC-0202236 | NA |
| 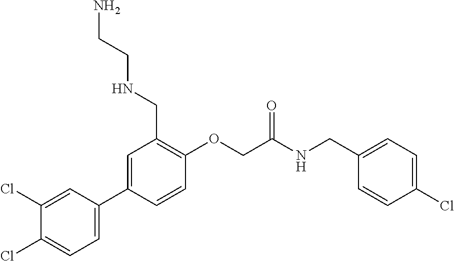 [SMILES: NCCNCc1cc(-c2ccc(Cl)c(Cl)c2)ccc1OCC(=O)NCc1ccc(Cl)cc1] | NUCC-0202237 | NA |
| 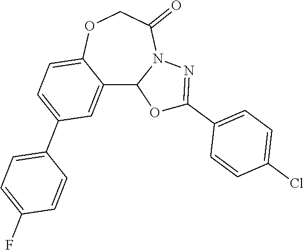 [SMILES: O=C1COc2ccc(-c3ccc(F)cc3)cc2C2OC(c3ccc(Cl)cc3)=NN12] | NUCC-0202238 | NA |
| 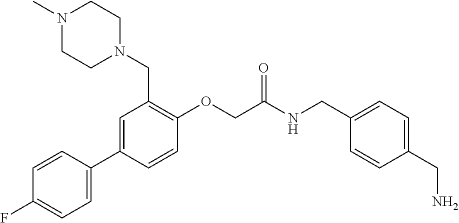 [SMILES: CN1CCN(Cc2cc(-c3ccc(F)cc3)ccc2OCC(=O)NCc2ccc(CN)cc2)CC1] | NUCC-0202239 | NA |
| 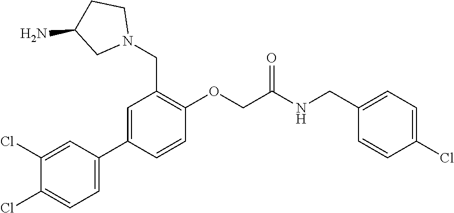 [SMILES: N[C@H]1CCN(Cc2cc(-c3ccc(Cl)c(Cl)c3)ccc2OCC(=O)NCc2ccc(Cl)cc2)C1] | NUCC-0202240 | NA |
| 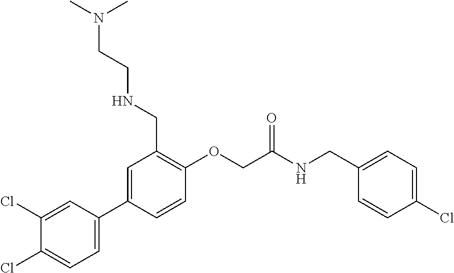 [SMILES: CN(C)CCNCc1cc(-c2ccc(Cl)c(Cl)c2)ccc1OCC(=O)NCc1ccc(Cl)cc1] | NUCC-0202384 | NA |
| 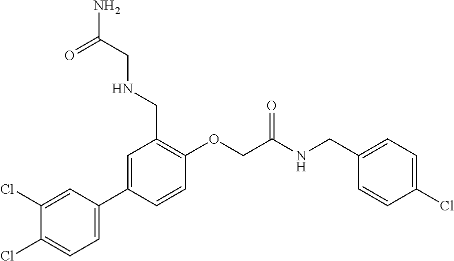 [SMILES: NC(=O)CNCc1cc(-c2ccc(Cl)c(Cl)c2)ccc1OCC(=O)NCc1ccc(Cl)cc1] | NUCC-0202385 | NA |
| 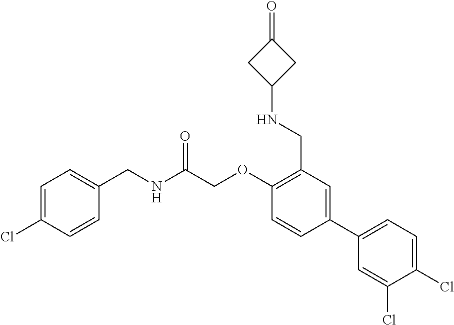 [SMILES: O=C1CC(NCc2cc(-c3ccc(Cl)c(Cl)c3)ccc2OCC(=O)NCc2ccc(Cl)cc2)C1] | NUCC-0202386 | NA |
| 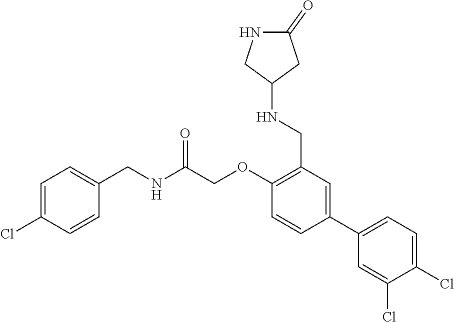 [SMILES: O=C(COc1ccc(-c2ccc(Cl)c(Cl)c2)cc1CNC1CNC(=O)C1)NCc1ccc(Cl)cc1] | NUCC-0202387 | NA |
| 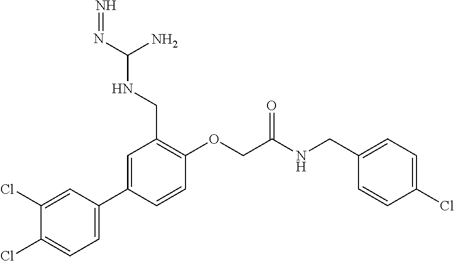 [SMILES: N=NC(N)NCc1cc(-c2ccc(Cl)c(Cl)c2)ccc1OCC(=O)NCc1ccc(Cl)cc1] | NUCC-0202388 | NA |
| 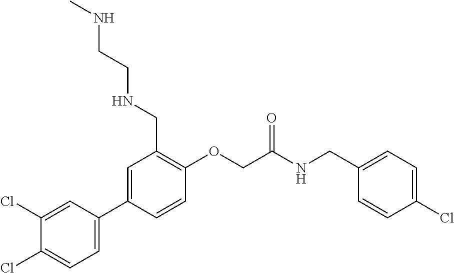 [SMILES: CNCCNCc1cc(-c2ccc(Cl)c(Cl)c2)ccc1OCC(=O)NCc1ccc(Cl)cc1] | NUCC-0202390 | NA |
| 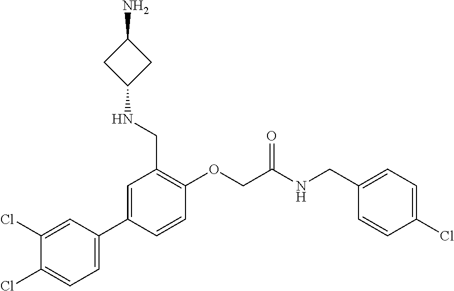 [SMILES: N[C@H]1C[C@H](NCc2cc(-c3ccc(Cl)c(Cl)c3)ccc2OCC(=O)NCc2ccc(Cl)cc2)C1] | NUCC-0202391 | NA |
| 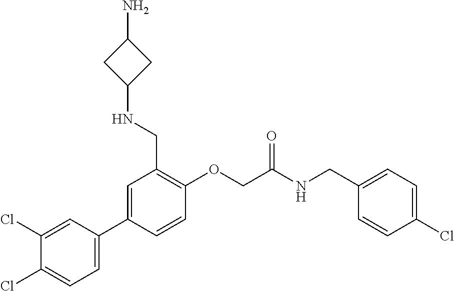 [SMILES: NC1CC(NCc2cc(-c3ccc(Cl)c(Cl)c3)ccc2OCC(=O)NCc2ccc(Cl)cc2)C1] | NUCC-0202392 | NA |
| 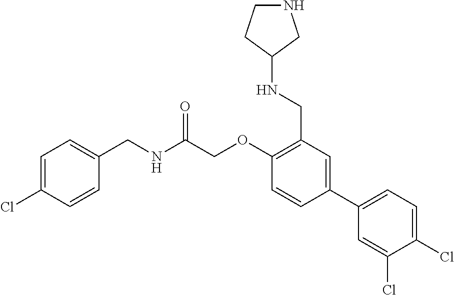 [SMILES: O=C(COc1ccc(-c2ccc(Cl)c(Cl)c2)cc1CNC1CCNC1)NCc1ccc(Cl)cc1] | NUCC-0202393 | NA |
| 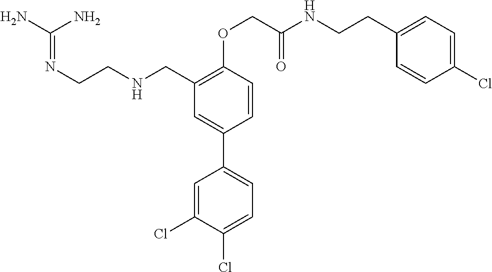 [SMILES: NC(N)=NCCNCc1cc(-c2ccc(Cl)c(Cl)c2)ccc1OCC(=O)NCCc1ccc(Cl)cc1] | NUCC-0202394 | NA |
| 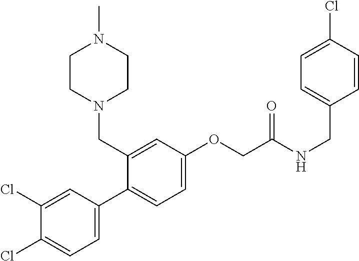 [SMILES: CN1CCN(Cc2cc(OCC(=O)NCc3ccc(Cl)cc3)ccc2-c2ccc(Cl)c(Cl)c2)CC1] | NUCC-0202396 | NA |
| 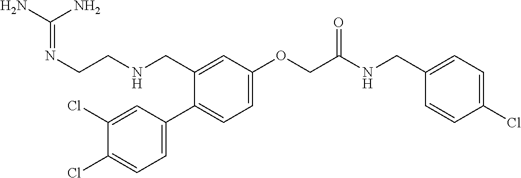 [SMILES: NC(N)=NCCNCc1cc(OCC(=O)NCc2ccc(Cl)cc2)ccc1-c1ccc(Cl)c(Cl)c1] | NUCC-0202397 | NA |
| 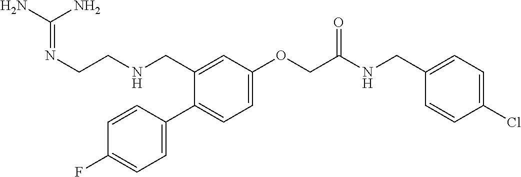 [SMILES: NC(N)=NCCNCc1cc(OCC(=O)NCc2ccc(Cl)cc2)ccc1-c1ccc(F)cc1] | NUCC-0202398 | NA |
| 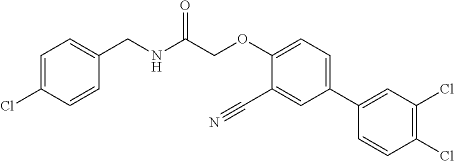 [SMILES: N#Cc1cc(-c2ccc(Cl)c(Cl)c2)ccc1OCC(=O)NCc1ccc(Cl)cc1] | NUCC-0202858 | NA |
| 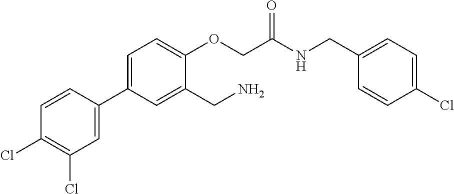 [SMILES: NCc1cc(-c2ccc(Cl)c(Cl)c2)ccc1OCC(=O)NCc1ccc(Cl)cc1] | NUCC-0202859 | NA |
| 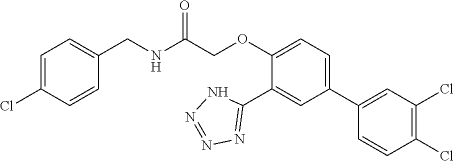 [SMILES: O=C(COc1ccc(-c2ccc(Cl)c(Cl)c2)cc1-c1nnn[nH]1)NCc1ccc(Cl)cc1] | NUCC-0202860 | NA |
| 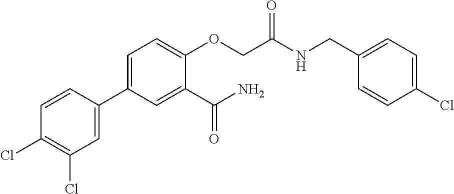 [SMILES: NC(=O)c1cc(-c2ccc(Cl)c(Cl)c2)ccc1OCC(=O)NCc1ccc(Cl)cc1] | NUCC-0202861 | NA |
| 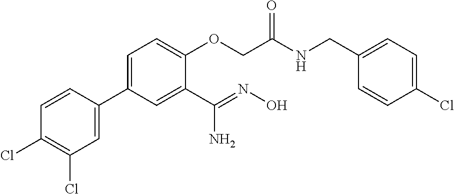 [SMILES: N/C(=N\O)c1cc(-c2ccc(Cl)c(Cl)c2)ccc1OCC(=O)NCc1ccc(Cl)cc1] | NUCC-0202862 | NA |
| 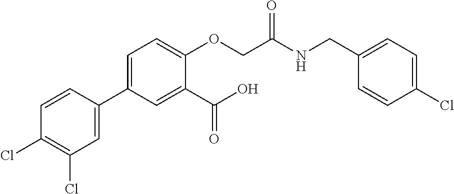 [SMILES: O=C(COc1ccc(-c2ccc(Cl)c(Cl)c2)cc1C(=O)O)NCc1ccc(Cl)cc1] | NUCC-0202863 | NA |
| 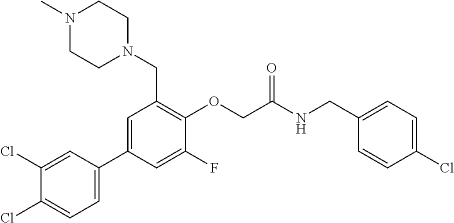 [SMILES: CN1CCN(Cc2cc(-c3ccc(Cl)c(Cl)c3)cc(F)c2OCC(=O)NCc2ccc(Cl)cc2)CC1] | NUCC-0202864 | NA |
| 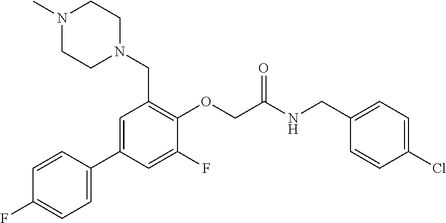 [SMILES: CN1CCN(Cc2cc(-c3ccc(F)cc3)cc(F)c2OCC(=O)NCc2ccc(Cl)cc2)CC1] | NUCC-0202865 | NA |
| 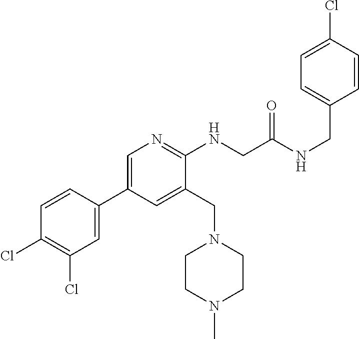 [SMILES: CN1CCN(Cc2cc(-c3ccc(Cl)c(Cl)c3)cnc2NCC(=O)NCc2ccc(Cl)cc2)CC1] | NUCC-0202866 | NA |
| 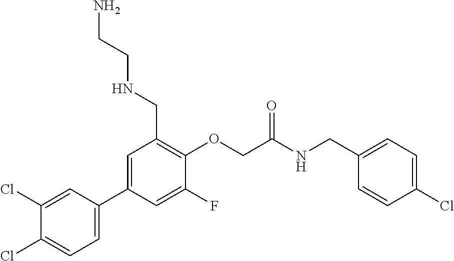 [SMILES: NCCNCc1cc(-c2ccc(Cl)c(Cl)c2)cc(F)c1OCC(=O)NCc1ccc(Cl)cc1] | NUCC-0202867 | NA |
| 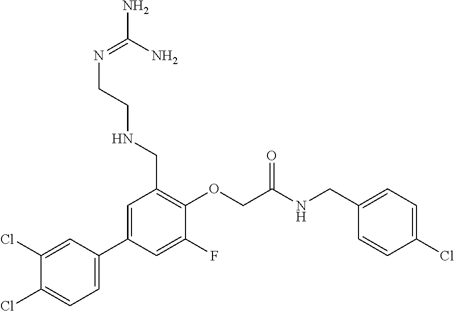 [SMILES: NC(N)=NCCNCc1cc(-c2ccc(Cl)c(Cl)c2)cc(F)c1OCC(=O)NCc1ccc(Cl)cc1] | NUCC-0202868 | NA |
| 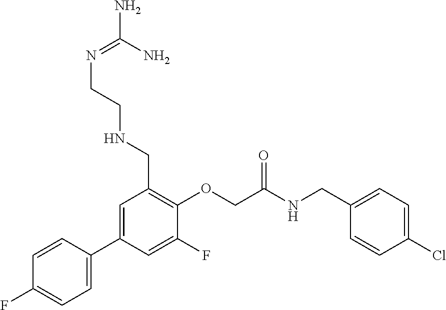 [SMILES: NC(N)=NCCNCc1cc(-c2ccc(F)cc2)cc(F)c1OCC(=O)NCc1ccc(Cl)cc1] | NUCC-0202869 | NA |
| 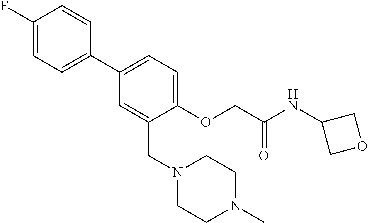 [SMILES: CN1CCN(Cc2cc(-c3ccc(F)cc3)ccc2OCC(=O)NC2COC2)CC1] | NUCC-0227513 | NA |
| 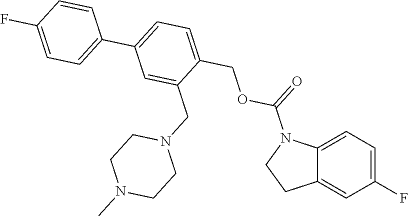 [SMILES: CN1CCN(Cc2cc(-c3ccc(F)cc3)ccc2COC(=O)N2CCc3cc(F)ccc32)CC1] | NUCC-0227512 | NA |
| 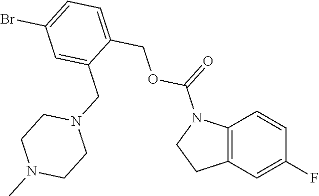 [SMILES: CN1CCN(Cc2cc(Br)ccc2COC(=O)N2CCc3cc(F)ccc32)CC1] | NUCC-0227511 | NA |
| 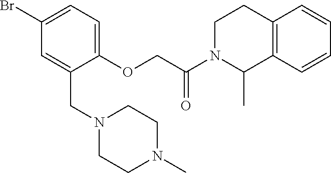 [SMILES: CC1c2ccccc2CCN1C(=O)COc1ccc(Br)cc1CN1CCN(C)CC1] | NUCC-0227510 | NA |
| 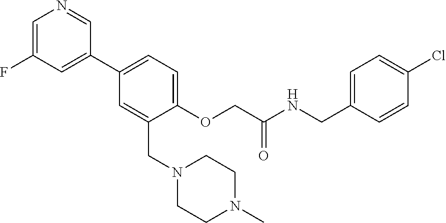 [SMILES: CN1CCN(Cc2cc(-c3cncc(F)c3)ccc2OCC(=O)NCc2ccc(Cl)cc2)CC1] | NUCC-0227423 | NA |
| 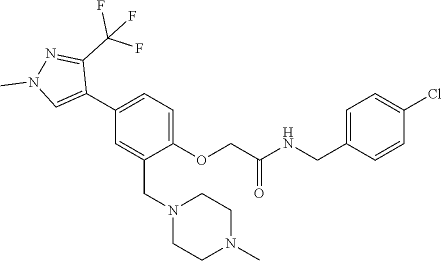 [SMILES: CN1CCN(Cc2cc(-c3cn(C)nc3C(F)(F)F)ccc2OCC(=O)NCc2ccc(Cl)cc2)CC1] | NUCC-0227422 | NA |
| 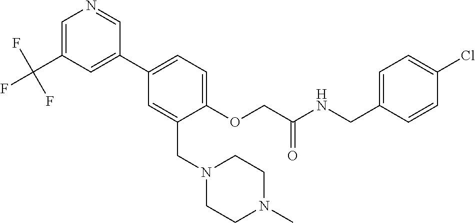 [SMILES: CN1CCN(Cc2cc(-c3cncc(C(F)(F)F)c3)ccc2OCC(=O)NCc2ccc(Cl)cc2)CC1] | NUCC-0227421 | NA |
| 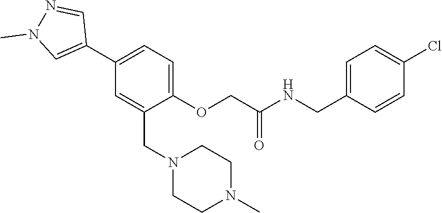 [SMILES: CN1CCN(Cc2cc(-c3cnn(C)c3)ccc2OCC(=O)NCc2ccc(Cl)cc2)CC1] | NUCC-0227420 | NA |
| 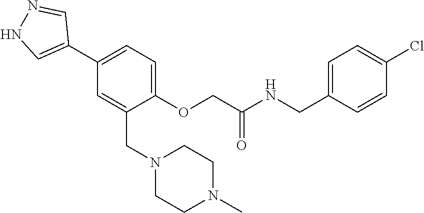 [SMILES: CN1CCN(Cc2cc(-c3cn[nH]c3)ccc2OCC(=O)NCc2ccc(Cl)cc2)CC1] | NUCC-0227419 | NA |
| 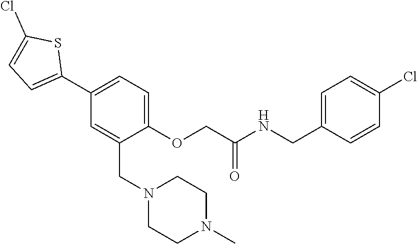 [SMILES: CN1CCN(Cc2cc(-c3ccc(Cl)s3)ccc2OCC(=O)NCc2ccc(Cl)cc2)CC1] | NUCC-0227418 | NA |
| 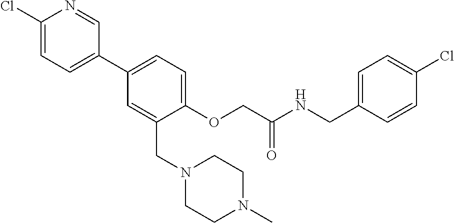 [SMILES: CN1CCN(Cc2cc(-c3ccc(Cl)nc3)ccc2OCC(=O)NCc2ccc(Cl)cc2)CC1] | NUCC-0227408 | NA |
| 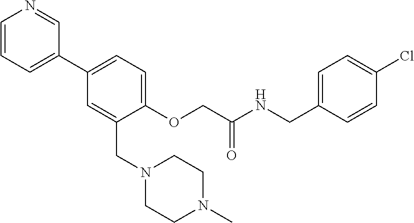 [SMILES: CN1CCN(Cc2cc(-c3cccnc3)ccc2OCC(=O)NCc2ccc(Cl)cc2)CC1] | NUCC-0227407 | NA |
| 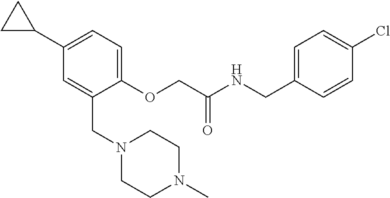 [SMILES: CN1CCN(Cc2cc(C3CC3)ccc2OCC(=O)NCc2ccc(Cl)cc2)CC1] | NUCC-0227406 | NA |

### REFERENCES

Airan R D, Meltzer L A, Roy M, Gong Y, Chen H, Deisseroth K (2007) High-Speed Imaging Reveals Neurophysiological Links to Behavior in an Animal Model of Depression. Science 317:815-819.
Anon (n.d.) Schrodinger LLC, N., Schrödinger Release 2014-4: LigPrep, version 3.2, Schrödinger, LLC, New York, NY, 2014. 2014.
Anon (n.d.) Schrodinger LLC, N. Small-Molecule Drug Discovery Suite 2014-4: Schrödinger Suite 2014-4 Induced Fit Docking protocol; Glide version 6.5, Schrödinger, LLC, New York, NY, 2014; Prime version 3.8, Schrödinger, LLC, New York, NY, 2014., 2014.
Baell J B, Holloway G A (2010) New substructure filters for removal of pan assay interference compounds (PAINS) from screening libraries and for their exclusion in bioassays. J Med Chem 53:2719-2740.
Bankston J R, Camp S S, DiMaio F (2012) Structure and stoichiometry of an accessory subunit TRIP8b interaction with hyperpolarization-activated cyclic nucleotide-gated channels.
Borer D D J (2008) Drugs 2007; 67 Suppl. 2: 15-24. :1-11.
Castagné V, Porsolt R D, Moser P (2009) Use of latency to immobility improves detection of antidepressant-like activity in the behavioral despair test in the mouse. Eur J Pharmacol 616:128-133.
Cross J B, Thompson D C, Rai B K, Baber J C, Fan K Y, Hu Y, Humblet C (n.d.) Comparison of Several Molecular Docking Programs: Pose Prediction and Virtual Screening Accuracy.
Daly E J, Singh J B, Fedgchin M, Cooper K, Lim P, Shelton R C, Thase M E, Winokur A, Van Nueten L, Manji H, Drevets W C (2018) Efficacy and Safety of Intranasal Esketamine Adjunctive to Oral Antidepressant Therapy in Treatment-Resistant Depression. JAMA Psychiatry 75:139.
Duman R S, Shinohara R, Fogaga M V, Hare B (2019) Neurobiology of rapid-acting antidepressants: convergent effects on GluA1-synaptic function. Molecular Psychiatry: 1-17.
Fisher D W, Han Y, Lyman K A, Heuermann R J, Bean L A, Ybarra N, Foote K M, Dong H, Nicholson D A, Chetkovich D M (2018) HCN channels in the hippocampus regulate active coping behavior. Journal of Neurochemistry 146:753-766.
Foote K M, Lyman K A, Han Y, Michailidis I E, Heuermann R J, Mandikian D, Trimmer J S, Swanson G T, Chetkovich D M (2019) Phosphorylation of the HCN channel auxiliary subunit TRIP8b is altered in an animal model of temporal lobe epilepsy and modulates channel function. Journal of Biological Chemistry:jbc.RA119.010027.
Halgren T A, Murphy R B, Friesner R A, Beard H S, Pollard W T, Banks J L (n.d.) Glide: A New Approach for Rapid, Accurate Docking and Scoring. 2. Enrichment Factors in Database Screening.
Han Y, Heuermann R J, Lyman K A, Fisher D, Ismail Q A, Chetkovich D M (2016a) HCN-channel dendritic targeting requires bipartite interaction with TRIP8b and regulates antidepressant-like behavioral effects. : 1-8.
Han Y, Lyman K, Clutter M, Schiltz G E, Ismail Q A, Prados D B, Luan C H, Chetkovich D M (2015) Identification of Small-Molecule Inhibitors of Hyperpolarization-Activated Cyclic Nucleotide-Gated Channels. J Biomol Screen 20:1124-1131.
Han Y, Lyman K A, Clutter M, Schiltz G E, Ismail Q-A, Cheng X, Luan C-H, Chetkovich D M (2016b) Method for Identifying Small Molecule Inhibitors of the Protein-protein Interaction Between HCN1 and TRIP8b. J Vis Exp:e54540-e54540.
Han Y, Noam Y, Lewis A S, Gallagher J J (2011) Trafficking and gating of hyperpolarization-activated cyclic nucleotide-gated channels are regulated by interaction with tetratricopeptide repeat-containing Rab8b- . . . . Journal of Biological . . . .
Herrmann S, Hofmann F, Stieber J, Ludwig A (2012) HCN channels in the heart: lessons from mouse mutants. Br J Pharmacol 166:501-509.
Heuermann R J, Jaramillo T C, Ying S-W, Suter B A, Lyman K A, Han Y, Lewis A S, Hampton T G, Shepherd G M G, Goldstein P A, Chetkovich D M (2016) Reduction of thalamic and cortical Ih by deletion of TRIP8b produces a mouse model of human absence epilepsy. Neurobiol Dis 85:81-92.
Hu L, Santoro B, Saponaro A, Liu H, Moroni A, Siegelbaum S A (2013) Binding of the auxiliary subunit TRIP8b to HCN channels shifts the mode of action of cAMP. The Journal of General Physiology.
Irwin J J, Shoichet B K (2005) ZINC-a free database of commercially available compounds for virtual screening. J Chem Inf Model 45:177-182.
Iyamu I D, Lv W, Malik N, Mishra R K, Schiltz G E (2019) Discovery of a novel class of potent and selective tetrahydroindazole-based sigma-1 receptor ligands. Bioorganic & Medicinal Chemistry 27:1824-1835.
Kessler R C, Bromet E J (2013) The Epidemiology of Depression Across Cultures. Annu Rev Public Health 34:119-138.
Kim C S, Brager D H, Johnston D (2017) Perisomatic changes in h-channels regulate depressive behaviors following chronic unpredictable stress. Molecular Psychiatry 18:7613.
Kim C S, Chang P Y, Johnston D (2012) Enhancement of dorsal hippocampal activity by knockdown of HCN1 channels leads to anxiolytic- and antidepressant-like behaviors. Neuron 75:503-516.
Kim C S, Johnston D (2020) Antidepressant Effects of (S)-Ketamine through a Reduction of Hyperpolarization-Activated Current Ih. iScience 23:101239.
Ku S M, Han M-H (2017) HCN Channel Targets for Novel Antidepressant Treatment. :1-18.
Lewis A S, Schwartz E, Chan C S, Noam Y, Shin M, Wadman W J, Surmeier D J, Baram T Z, Macdonald R L, Chetkovich D M (2009) Alternatively spliced isoforms of TRIP8b differentially control h channel trafficking and function. J Neurosci 29:6250-6265.
Lewis A S, Vaidya S P, Blaiss C A, Liu Z, Stoub T R, Brager D H, Chen X, Bender R A, Estep C M, Popov A B, Kang C E, Van Veldhoven P P, Bayliss D A, Nicholson D A, Powell C M, Johnston D, Chetkovich D M (2011) Deletion of the hyperpolarization-activated cyclic nucleotide-gated channel auxiliary subunit TRIP8b impairs hippocampal Ih localization and function and promotes antidepressant behavior in mice. J Neurosci 31:7424-7440.
Lyman K A, Han Y, Chetkovich D M (2017a) Animal models suggest the TRIP8b-HCN interaction is a therapeutic target for Major Depressive Disorder. Expert Opinion on Therapeutic Targets 0:1-13.
Lyman K A, Han Y, Heuermann R J, Cheng X, Kurz J E, Lyman R E, Van Veldhoven P P, Chetkovich D M (2017b) Allostery between two binding sites in the ion channel subunit TRIP8b confers binding specificity to HCN channels. J Biol Chem 292:17718-17730.
Marcelin B, Liu Z, Chen Y, Lewis A S, Becker A, McClelland S, Chetkovich D M, Migliore M, Baram T Z, Esclapez M, Bernard C (2012) Dorsoventral differences in intrinsic properties in developing CA1 pyramidal cells. J Neurosci 32:3736-3747.
M D JSK, M D A L, M D S P, M D VYR, M D SRD (2017a) The Clinical Use of Ivabradine. Journal of the American College of Cardiology 70:1777-1784.
M D S K, M D H C, M D H G-B, Raines S, D O R A, PhD A S, PhD J D, M D PCNE, M D KMD, M D R R, Hoffmann E, M D PDR, M D J J, M D S P, M D DSM-B (2017b) Brexanolone (SAGE-547 injection) in post-partum depression: a randomised controlled trial. Lancet 390:480-489.
M D S M-B, M D H C, M D R R, M D PCNE, M D KMD, M D PDRR, PhD H L, PhD AJS, PhD C C, PhD A S, M D J J, M D S K (2018) Brexanolone injection in post-partum depression: two multicentre, double-blind, randomised, placebo-controlled, phase 3 trials. Lancet 392:1058-1070.
Pham T A, Jain A N (n.d.) Parameter Estimation for Scoring Protein-Ligand Interactions Using Negative Training Data.
Piskorowski R, Santoro B, Siegelbaum S A (2011) TRIP8b splice forms act in concert to regulate the localization and expression of HCN1 channels in CA1 pyramidal neurons. Neuron.
Ramirez D, Zúñiga R, Concha G, Znniga L (2018) HCN Channels: New Therapeutic Targets for Pain Treatment. Molecules 23:2094.
Robinson R B, Siegelbaum S A (2003) Hyperpolarization-Activated Cation Currents: From Molecules to Physiological Function. Annu Rev Physiol 65:453-480.
Santoro B, Hu L, Liu H, Saponaro A, Pian P, Piskorowski R A, Moroni A, Siegelbaum S A (2011) TRIP8b regulates HCN1 channel trafficking and gating through two distinct C-terminal interaction sites. J Neurosci 31:4074-4086.
Santoro B, Piskorowski R A, Pian P, Hu L, Liu H (2009) TRIP8b splice variants form a family of auxiliary subunits that regulate gating and trafficking of HCN channels in the brain. Neuron.
Santoro B, Wainger B J, Siegelbaum S A (2004) Regulation of HCN channel surface expression by a novel C-terminal protein-protein interaction. J Neurosci 24:10750-10762.
Saponaro A, Cantini F, Porro A, Bucchi A, DiFrancesco D, Maione V, Donadoni C, Introini B, Mesirca P, Mangoni M E, Thiel G, Banci L, Santoro B, Moroni A (2018) A synthetic peptide that prevents cAMP regulation in mammalian Hyperpolarization-activated Cyclic Nucleotide-regulated (HCN) channels. eLife Sciences 7:1-53.
Sartiani L, Mannaioni G, Massi A, Romanelli M N, Cerbai E (2017) The Hyperpolarization-Activated Cyclic Nucleotide-Gated Channels: from Biophysics to Pharmacology of a Unique Family of Ion Channels. Pharmacol Rev 69:1-42.
Stepan J, Hladky F, Uribe A, Holsboer F, Schmidt M V, Eder M (2015) High-Speed imaging reveals opposing effects of chronic stress and antidepressants on neuronal activity propagation through the hippocampal trisynaptic circuit. Front Neural Circuits 9:819.
Thompson S M, Kallarackal A J, Kvarta M D, Van Dyke A M, LeGates T A, Cai X (2015) An excitatory synapse hypothesis of depression. Trends Neurosci 38:279-294.
Tibbs G R, Posson D J, Goldstein P A (2016) Voltage-Gated Ion Channels in the PNS: Novel Therapies for Neuropathic Pain? Trends Pharmacol Sci: 1-21.
Wahl-Schott C, Biel M (2009) HCN channels: structure, cellular regulation and physiological function. Cellular and molecular life sciences.
Zhou C, Ding L, Deel M E, Ferrick E A, Emeson R B, Gallagher M J (2015) Altered intrathalamic GABAA neurotransmission in a mouse model of a human genetic absence epilepsy syndrome. Neurobiol Dis 73:407-417.
Zhou C, Huang Z, Ding L, Deel M E, Arain F M, Murray C R, Patel R S, Flanagan C D, Gallagher M J (2013) Altered Cortical GABA AReceptor Composition, Physiology, and Endocytosis in a Mouse Model of a Human Genetic Absence Epilepsy Syndrome. Journal of Biological Chemistry 288:21458-21472.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A compound of the following formula: 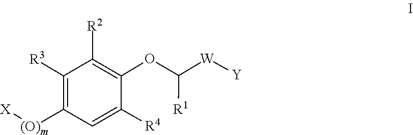
or a pharmaceutically acceptable salt thereof, wherein:
X is phenyl optionally substituted at one or more positions with halogen, alkyl, alkoxy, hydroxyl, carboxamido, hydroxyl, cyano, nitro, haloalkyl, alkylthio, alkenyl, amino, alkylsulfonyl, hydroxyalkyl, or phenyl optionally substituted at one or more positions with halogen;
or X is pyrimidinyl optionally substituted at one or more positions with alkoxy;
or X is pyridinyl optionally substituted at one or more positions with halogen or haloalkyl;
or X is pyrazolyl optionally substituted at one or more positions with positions with alkyl or haloalkyl;
or X is thiophenyl optionally substituted at one or more positions with halogen;
or X is cycloalkyl;
or X is halogen or haloalkyl;
m is 0 or 1;
R¹ is H or alkyl;
W is —CH₂— or —C(O)—;
Y is —NH-(Alk)ₙ-Z or —N(R⁵)—(Alk)ₙ-Z, wherein Alk is —CH₂— or —CH(CH₃)—, R⁵ is methyl or together with R⁴ forms a hetrocycle, and n is 0-2;
or Y is 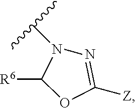
wherein R⁶ together with R⁴ forms a hetrocycle;
or Y is —NH—S(O)₂—Z;
or Y is carboxyl, amino, alkylamino, dialkylamino, indolinyl optionally substituted at one or more positions with halogen; indanyl, 1,2,3,4-tetrahydroisoquinolinyl, or isoindolinyl;
Z is phenyl optionally substituted at one or more positions with halogen, alkyl, haloalkyl, aminosulfonyl, alkoxy, pyrazolyl, imidazolyl, alkylsufonyl, alkylaminocarbonyl, hydroxyl, cyano, nitro, alkenyl, aminoalkyl, or hydroxyalkyl;
or Z is 1,3-benzodioxole; piperidinyl; pyridinyl optionally substituted with alkoxy or benzothiazole; indolinyl optionally substituted with halogen or alkyl; cycloalkyl; or a cyclic ether.
R² is —CH₂—Het, wherein Het is a saturated heterocycle comprising 5, 6, or 7 atoms wherein at least one of the atoms is a nitrogen atom, and the heterocycle is optionally substituted at one or more positions with alkyl, amino, alkylamino, dialkylamino, alkoxycarbonyl, or alkylsulfonyl;
or R² is H, —CH₂—NH₂, —C(O)—NH₂, —C(O)—OH, —C(NH₂)═N—OH, —CH₂—NH—CH₂—CH₂—N═C(NH₂)₂, —CH₂—NH—CH₂—CH₂—NH₂, —CH₂—NH—CH₂—CH₂—N(CH₃)₂, —CH₂—NH—CH₂—C(O)—NH₂, —CH₂—NH—CH₂—CH₂—NH—CH₃, —CH₂—NH—CH₂—CH₂—N═C(NH₂)₂, or —CH₂—NH—CH(NH₂)—N═NH;
or R² is 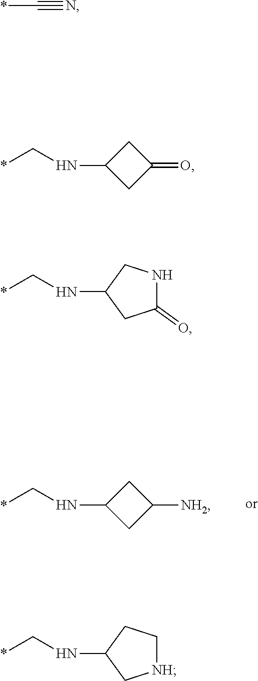
R³ is H or —CH₂—NH—CH₂—CH₂—N═C(NH₂)₂; and
R⁴ is H, —CH₂—NH₂, —C(O)—NH₂, or —C(NH₂)═N—OH.

2. The compound of claim 1, wherein X is phenyl optionally substituted at one or more positions with halogen, alkyl, alkoxy, hydroxyl, carboxamido, hydroxyl, cyano, nitro, haloalkyl, alkylthio, alkenyl, amino, alkylsulfonyl, hydroxyalkyl, or phenyl optionally substituted at one or more positions with halogen.

3. The compound of claim 1, wherein X is phenyl substituted at one or more positions with halogen.

4. The compound of claim 1, wherein m is 0.

5. The compound of claim 1, wherein R¹ is H.

6. The compound of claim 1, wherein W is —C(O)—.

7. The compound of claim 1, wherein Y is —NH-(Alk)ₙ-Z or —N(CH₃)—(Alk)ₙ-Z, wherein Alk is —CH₂— or —CH(CH₃)—, and n is 0-2.

8. The compound of claim 1, wherein Z is phenyl optionally substituted at one or more positions with halogen, alkyl, haloalkyl, aminosulfonyl, alkoxy, pyrazolyl, imidazolyl, alkylsufonyl, alkylaminocarbonyl, hydroxyl, cyano, nitro, alkenyl, aminoalkyl, or hydroxyalkyl.

9. The compound of claim 1, wherein Z is phenyl substituted at one or more positions with halogen.

10. The compound of claim 1, wherein R² is —CH₂—Het, wherein Het is a saturated heterocycle comprising 5, 6, or 7 atoms wherein at least one of the atoms is a nitrogen atom, and the heterocycle is optionally substituted at one or more positions with alkyl, amino, alkylamino, dialkylamino, alkoxycarbonyl, or alkylsulfonyl.

11. The compound of claim 1, wherein R² is —CH₂—Het, wherein Het is piperazinyl or piperidinyl, optionally substituted at one or more positions with alkyl, amino, alkylamino, dialkylamino, alkoxycarbonyl, or alkylsulfonyl.

12. The compound of claim 1, wherein R² is —CH₂-(4-methylpiperazinyl).

13. The compound of claim 1, wherein R³ is H.

14. The compound of claim 1, wherein R⁴ is H.

15. The compound of claim 1 selected from the group consisting of:

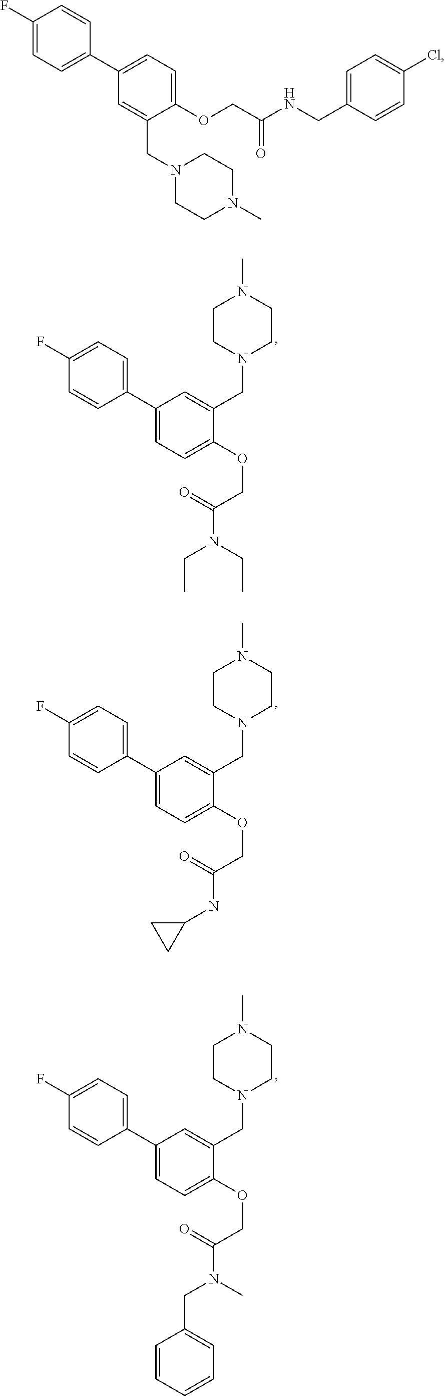

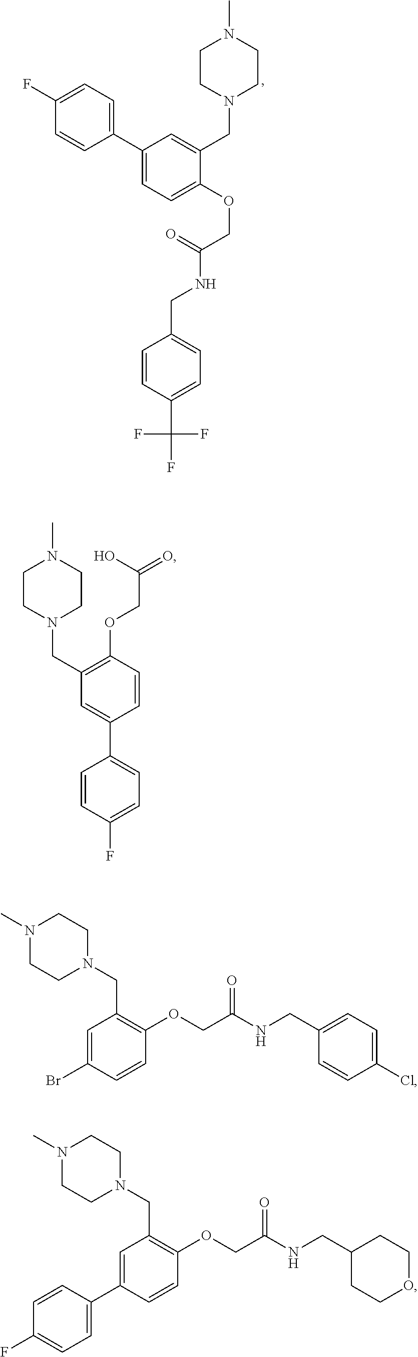

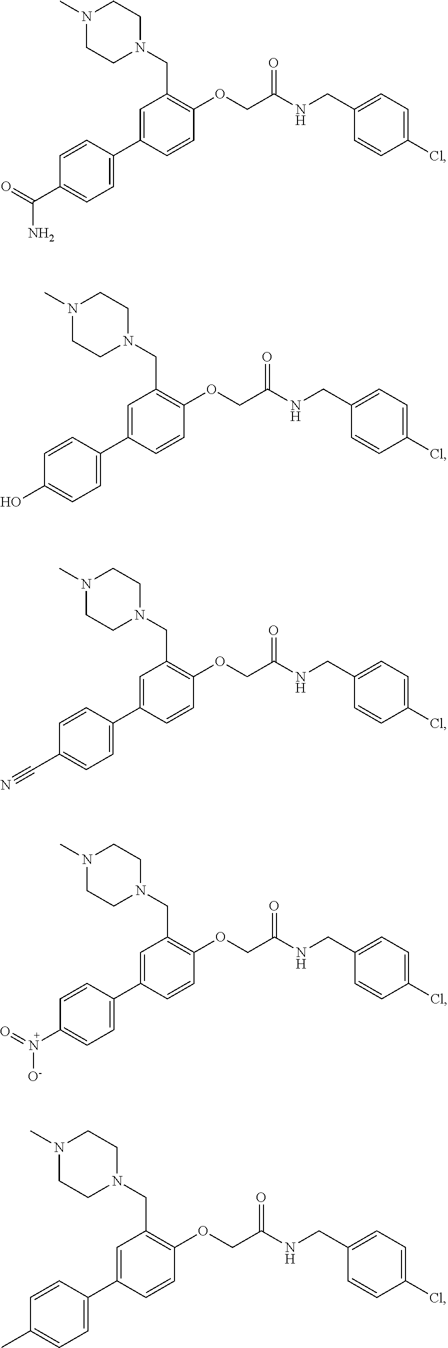

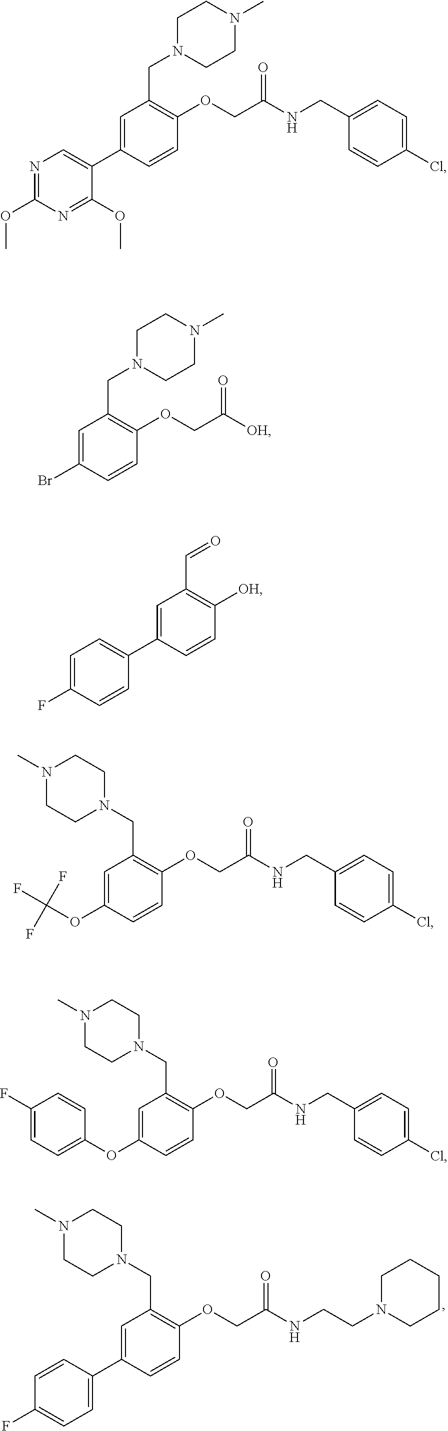 [SMILES: COc1ncc(-c2ccc(OCC(=O)NCc3ccc(Cl)cc3)c(CN3CCN(C)CC3)c2)c(OC)n1 ; CN1CCN(Cc2cc(Br)ccc2OCC(=O)O)CC1 ; O=Cc1cc(-c2ccc(F)cc2)ccc1O ; CN1CCN(Cc2cc(OC(F)(F)F)ccc2OCC(=O)NCc2ccc(Cl)cc2)CC1 ; CN1CCN(Cc2cc(Oc3ccc(F)cc3)ccc2OCC(=O)NCc2ccc(Cl)cc2)CC1 ; CN1CCN(Cc2cc(-c3ccc(F)cc3)ccc2OCC(=O)NCCN2CCCCC2)CC1]

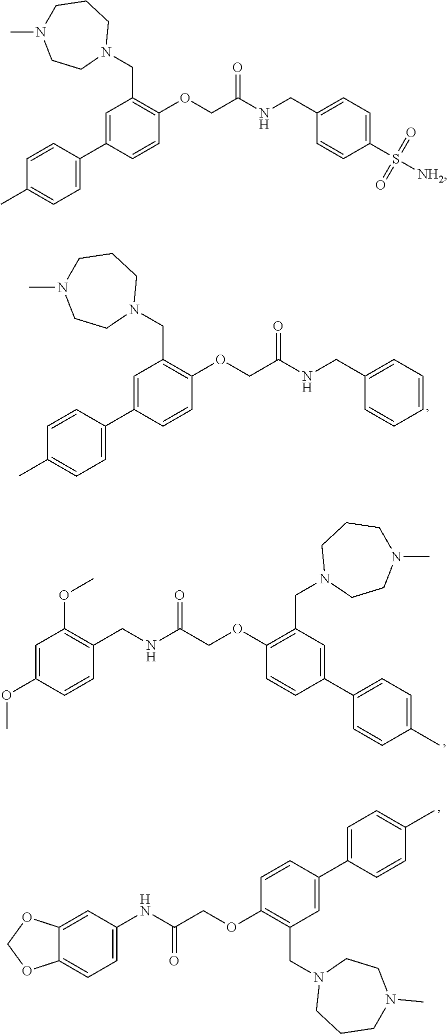

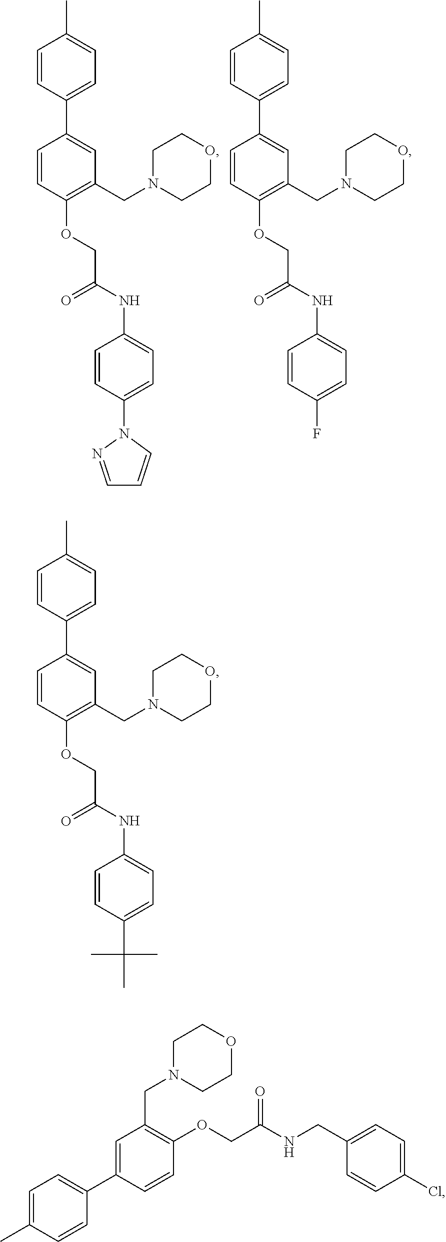

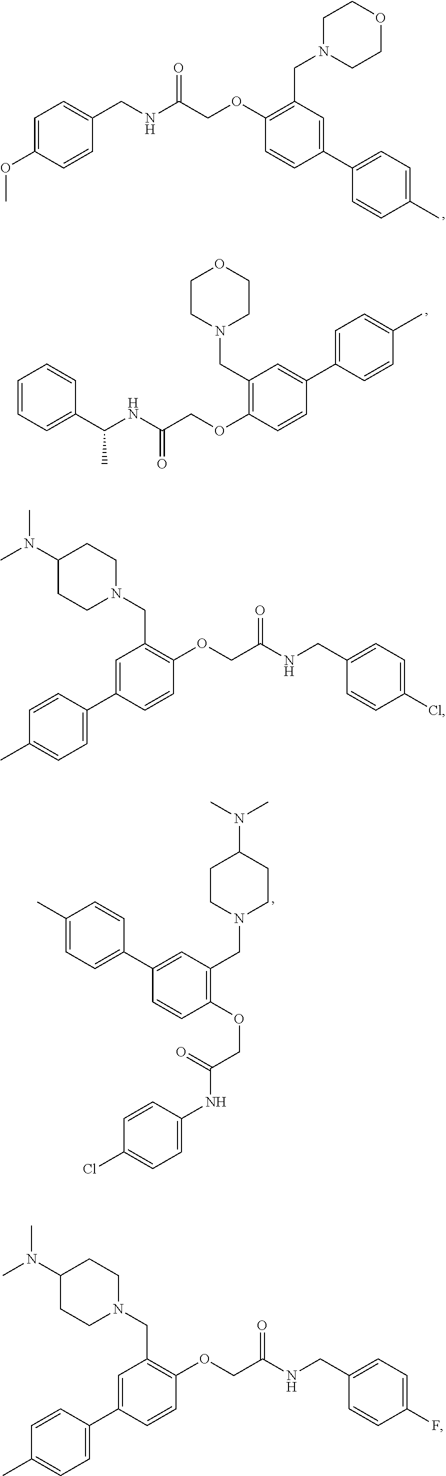

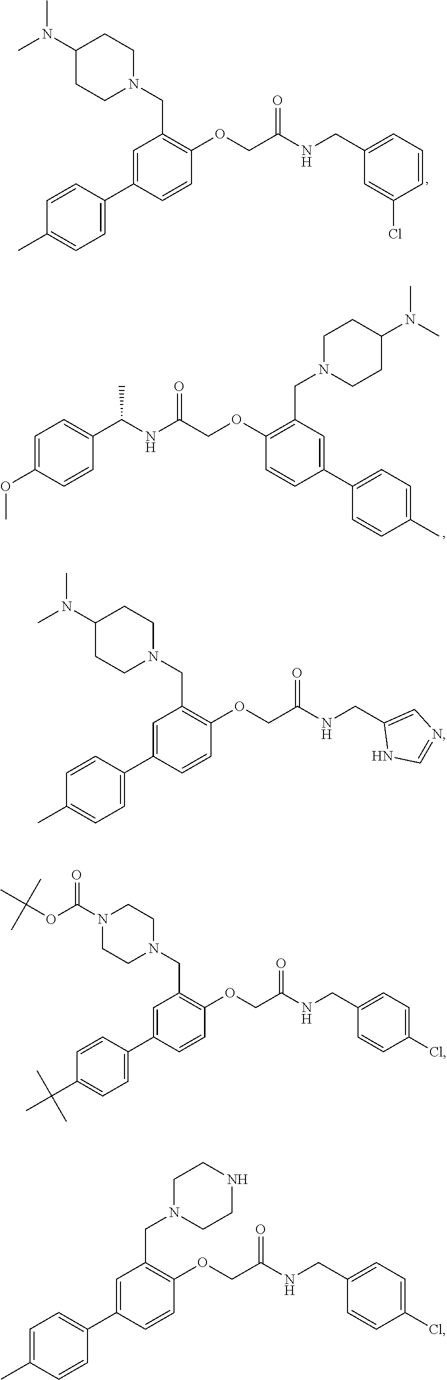

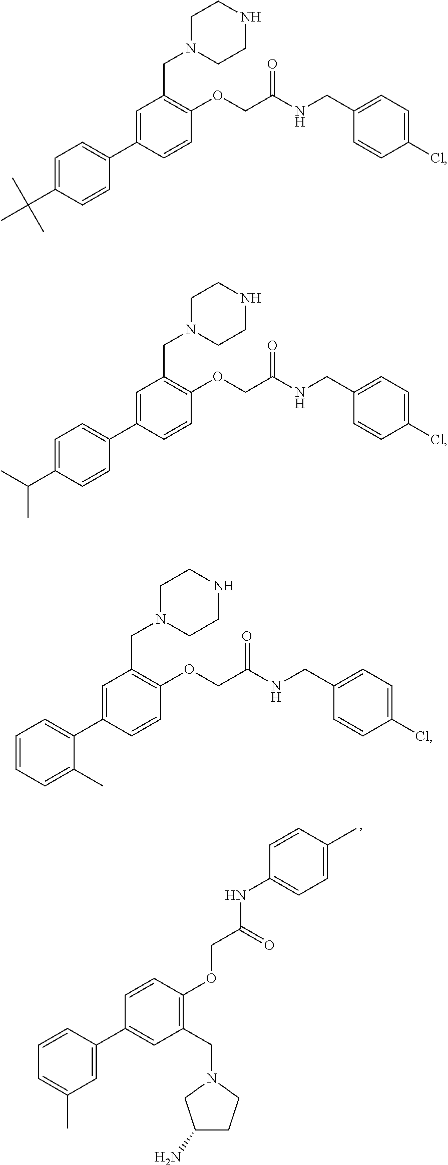 [SMILES: CC(C)(C)c1ccc(-c2ccc(OCC(=O)NCc3ccc(Cl)cc3)c(CN3CCNCC3)c2)cc1 ; CC(C)c1ccc(-c2ccc(OCC(=O)NCc3ccc(Cl)cc3)c(CN3CCNCC3)c2)cc1 ; Cc1ccccc1-c1ccc(OCC(=O)NCc2ccc(Cl)cc2)c(CN2CCNCC2)c1 ; Cc1ccc(NC(=O)COc2ccc(-c3cccc(C)c3)cc2CN2CC[C@H](N)C2)cc1]

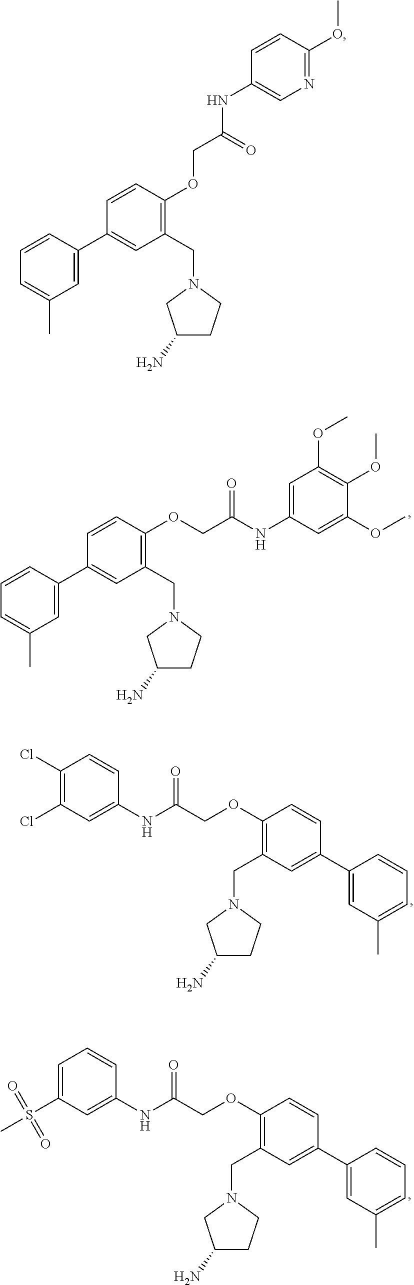

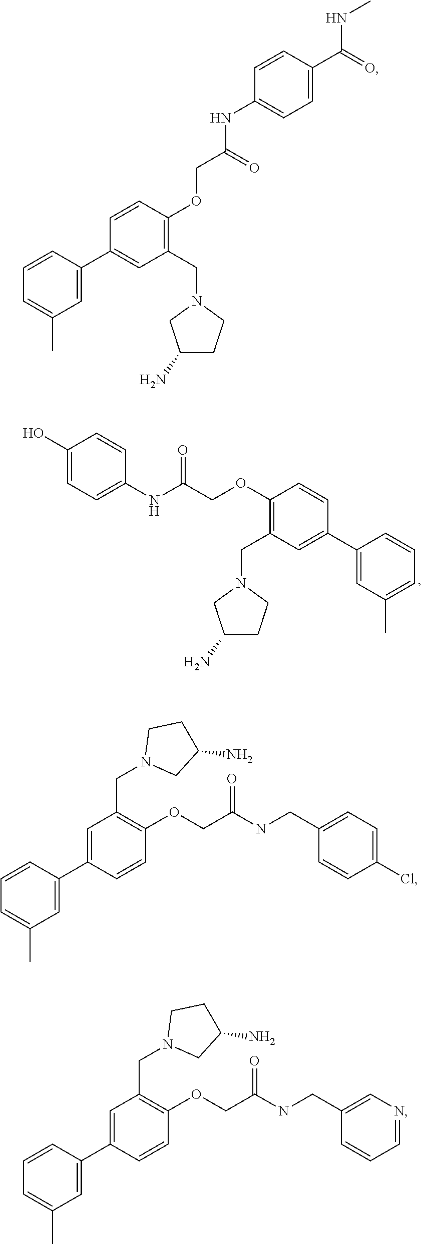

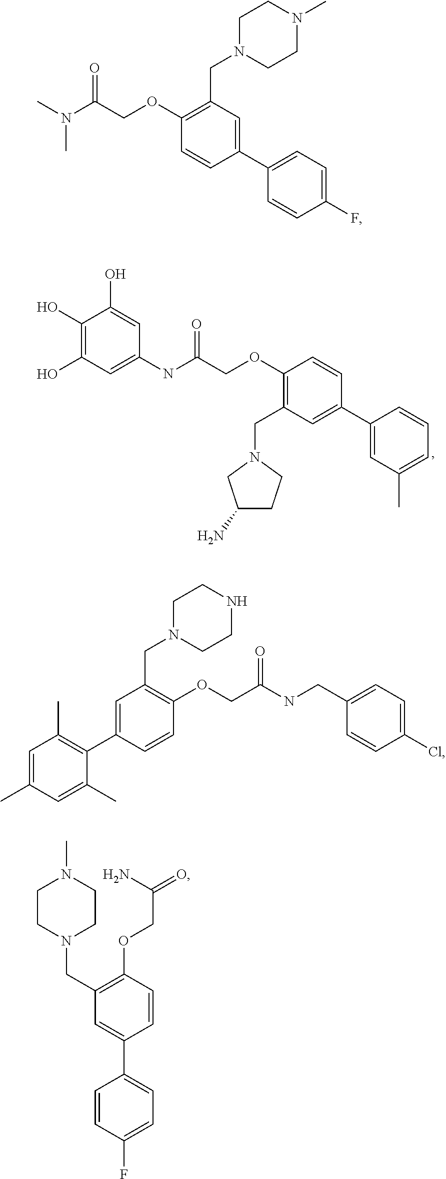

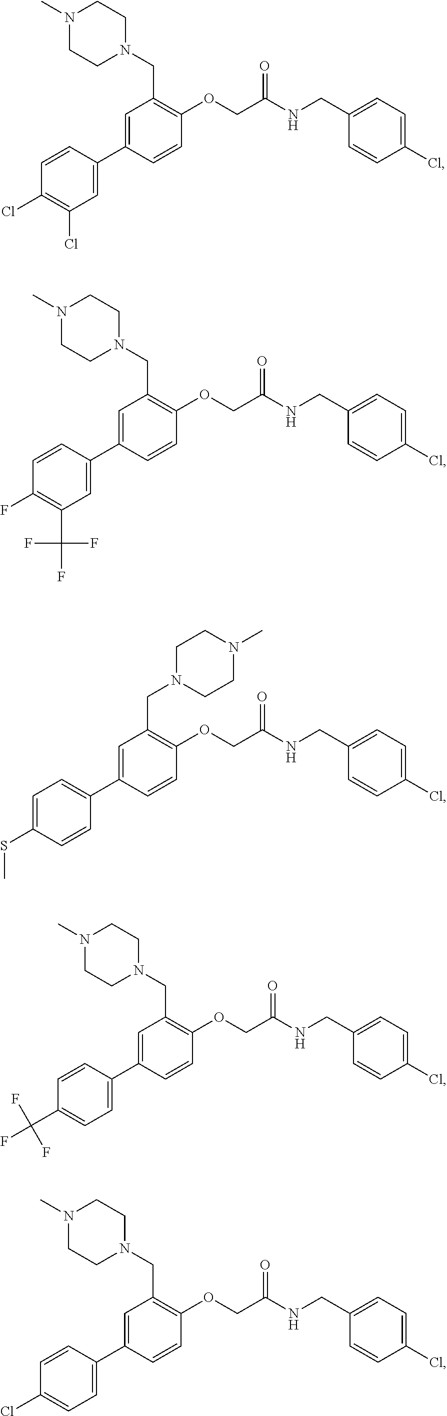 [SMILES: CN1CCN(Cc2cc(-c3ccc(Cl)c(Cl)c3)ccc2OCC(=O)NCc2ccc(Cl)cc2)CC1 ; CN1CCN(Cc2cc(-c3ccc(F)c(C(F)(F)F)c3)ccc2OCC(=O)NCc2ccc(Cl)cc2)CC1 ; CSc1ccc(-c2ccc(OCC(=O)NCc3ccc(Cl)cc3)c(CN3CCN(C)CC3)c2)cc1 ; CN1CCN(Cc2cc(-c3ccc(C(F)(F)F)cc3)ccc2OCC(=O)NCc2ccc(Cl)cc2)CC1 ; CN1CCN(Cc2cc(-c3ccc(Cl)cc3)ccc2OCC(=O)NCc2ccc(Cl)cc2)CC1]

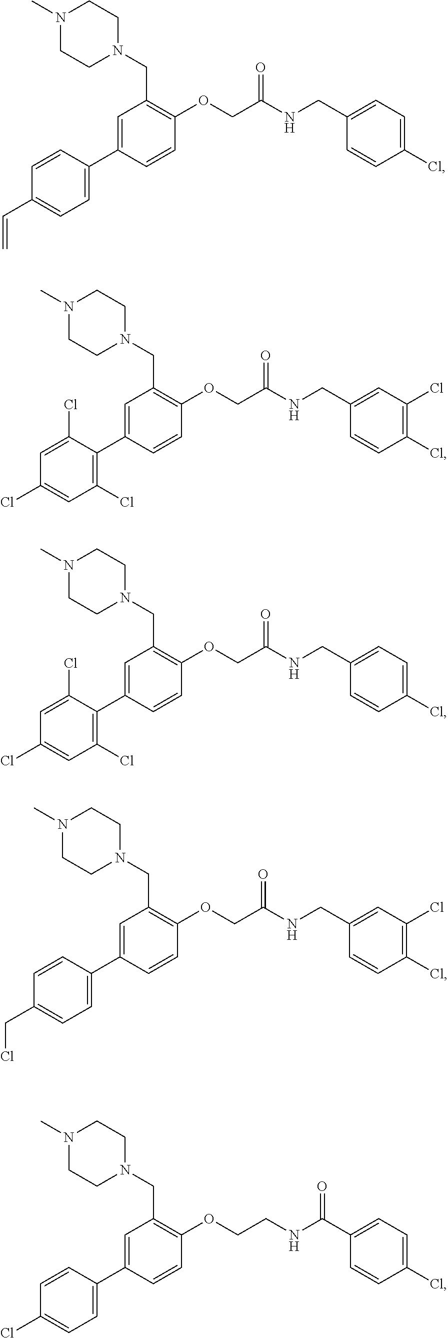 [SMILES: C=Cc1ccc(-c2ccc(OCC(=O)NCc3ccc(Cl)cc3)c(CN3CCN(C)CC3)c2)cc1 ; CN1CCN(Cc2cc(-c3c(Cl)cc(Cl)cc3Cl)ccc2OCC(=O)NCc2ccc(Cl)c(Cl)c2)CC1 ; CN1CCN(Cc2cc(-c3c(Cl)cc(Cl)cc3Cl)ccc2OCC(=O)NCc2ccc(Cl)cc2)CC1 ; CN1CCN(Cc2cc(-c3ccc(CCl)cc3)ccc2OCC(=O)NCc2ccc(Cl)c(Cl)c2)CC1 ; CN1CCN(Cc2cc(-c3ccc(Cl)cc3)ccc2OCCNC(=O)c2ccc(Cl)cc2)CC1]

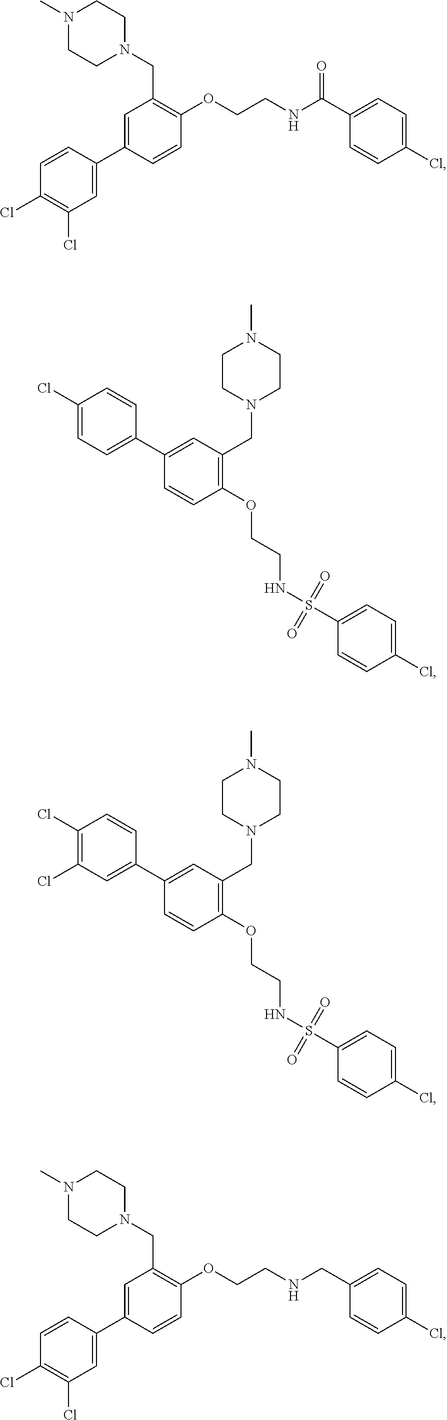

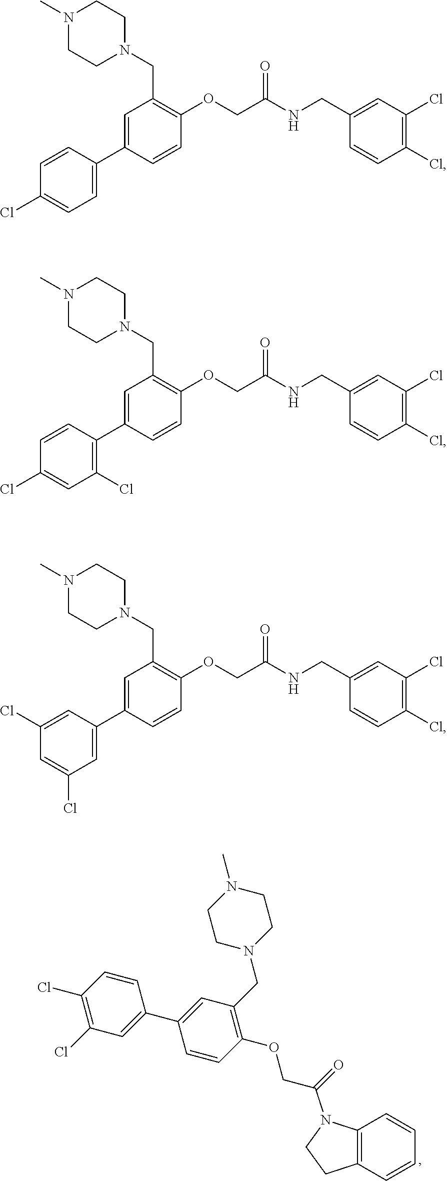 [SMILES: CN1CCN(Cc2cc(-c3ccc(Cl)cc3)ccc2OCC(=O)NCc2ccc(Cl)c(Cl)c2)CC1 ; CN1CCN(Cc2cc(-c3ccc(Cl)cc3Cl)ccc2OCC(=O)NCc2ccc(Cl)c(Cl)c2)CC1 ; CN1CCN(Cc2cc(-c3cc(Cl)cc(Cl)c3)ccc2OCC(=O)NCc2ccc(Cl)c(Cl)c2)CC1 ; CN1CCN(Cc2cc(-c3ccc(Cl)c(Cl)c3)ccc2OCC(=O)N2CCc3ccccc32)CC1]

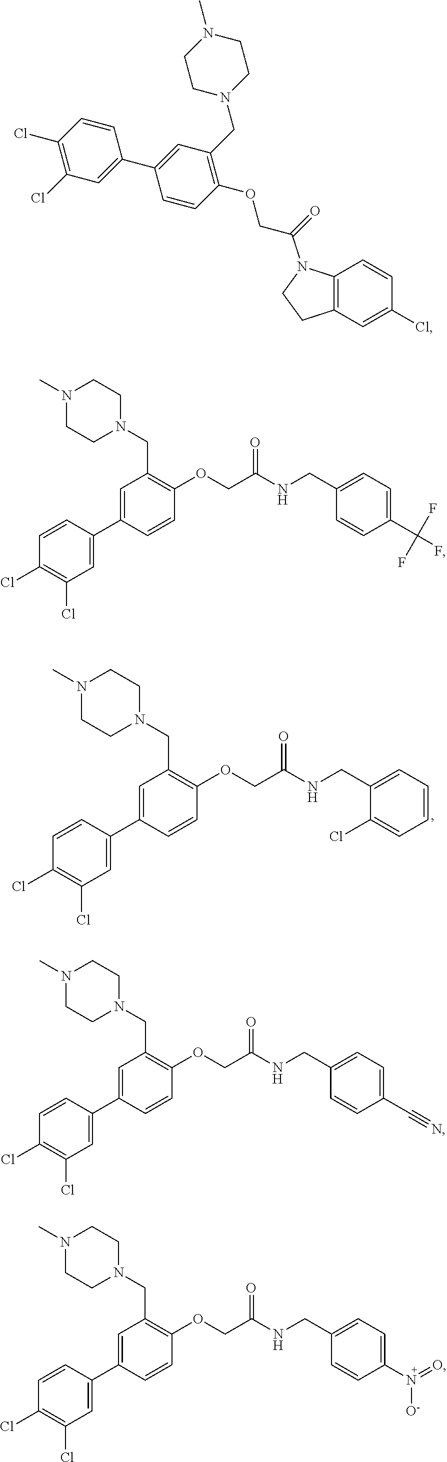

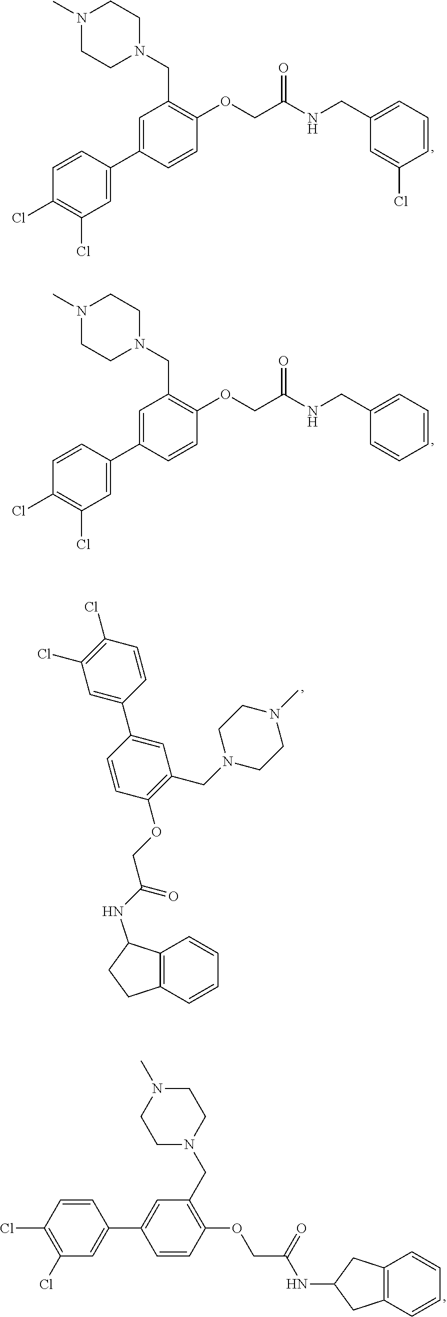 [SMILES: CN1CCN(Cc2cc(-c3ccc(Cl)c(Cl)c3)ccc2OCC(=O)NCc2cccc(Cl)c2)CC1 ; CN1CCN(Cc2cc(-c3ccc(Cl)c(Cl)c3)ccc2OCC(=O)NCc2ccccc2)CC1 ; CN1CCN(Cc2cc(-c3ccc(Cl)c(Cl)c3)ccc2OCC(=O)NC2CCc3ccccc32)CC1 ; CN1CCN(Cc2cc(-c3ccc(Cl)c(Cl)c3)ccc2OCC(=O)NC2Cc3ccccc3C2)CC1]

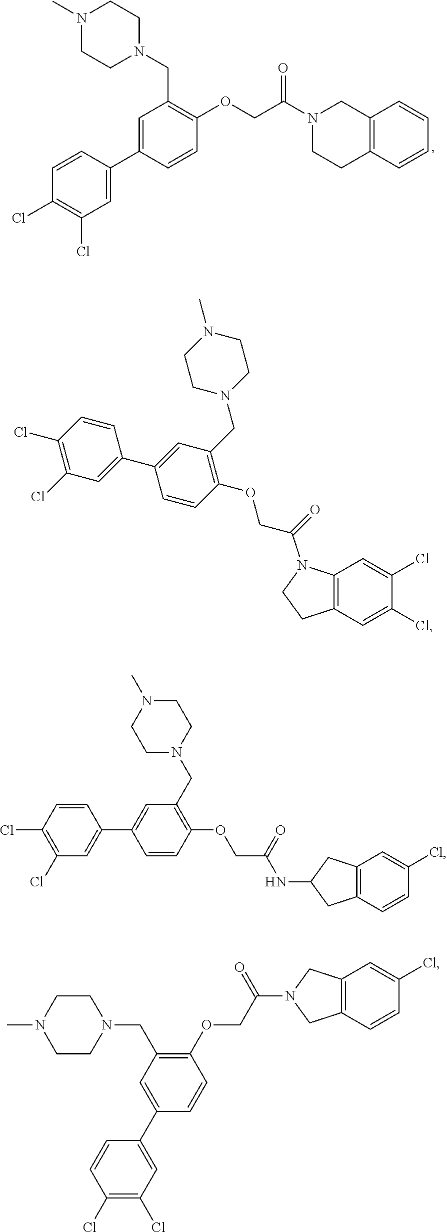

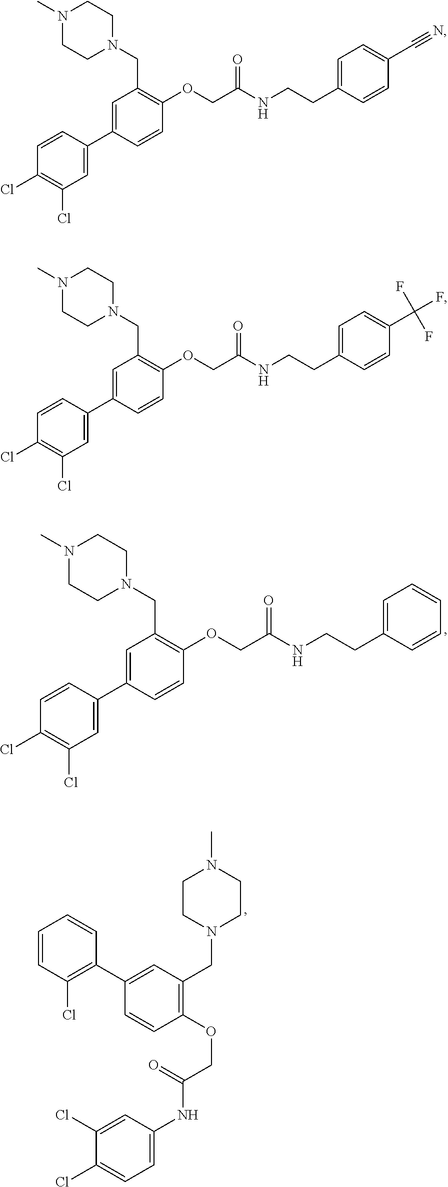

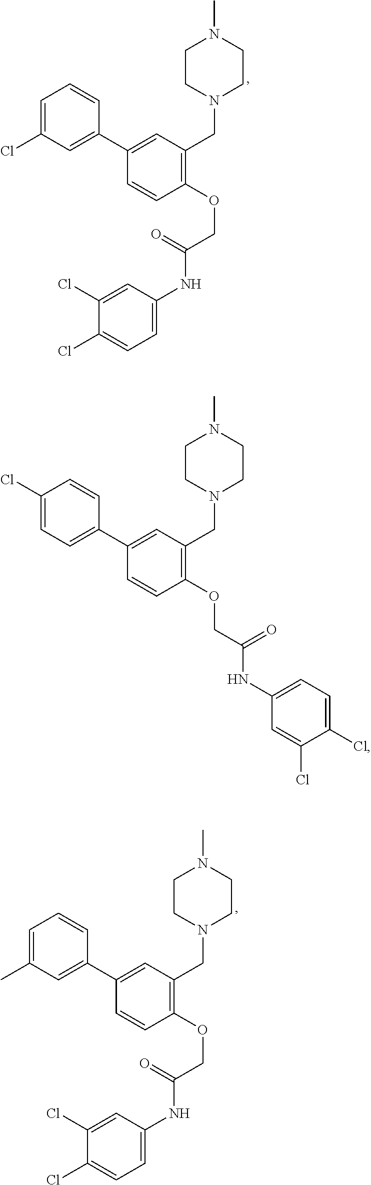

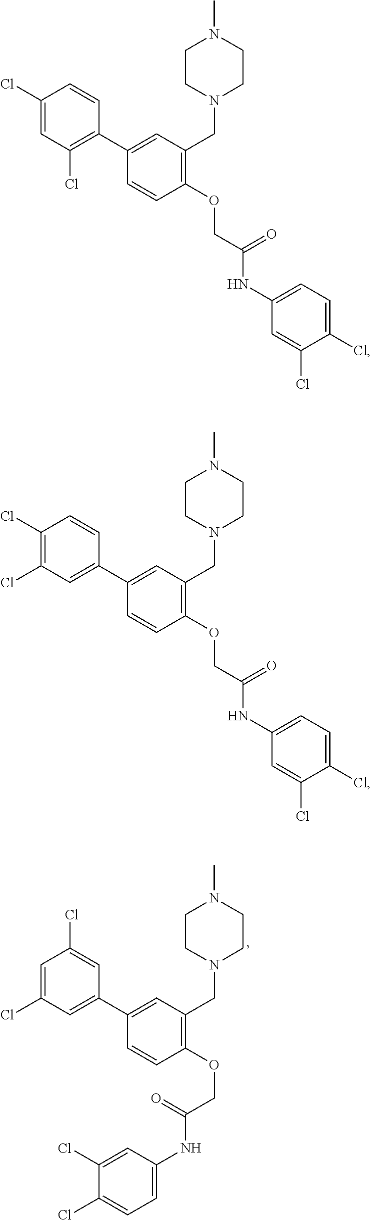

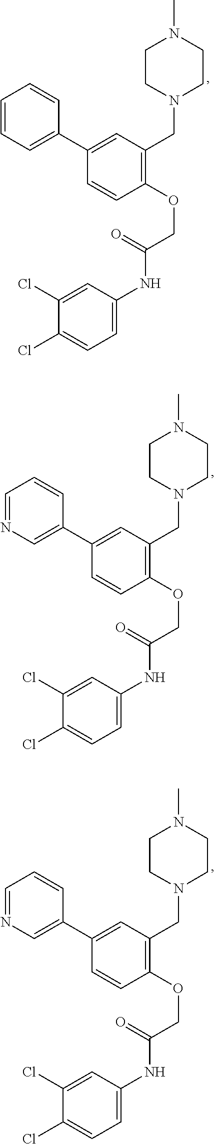

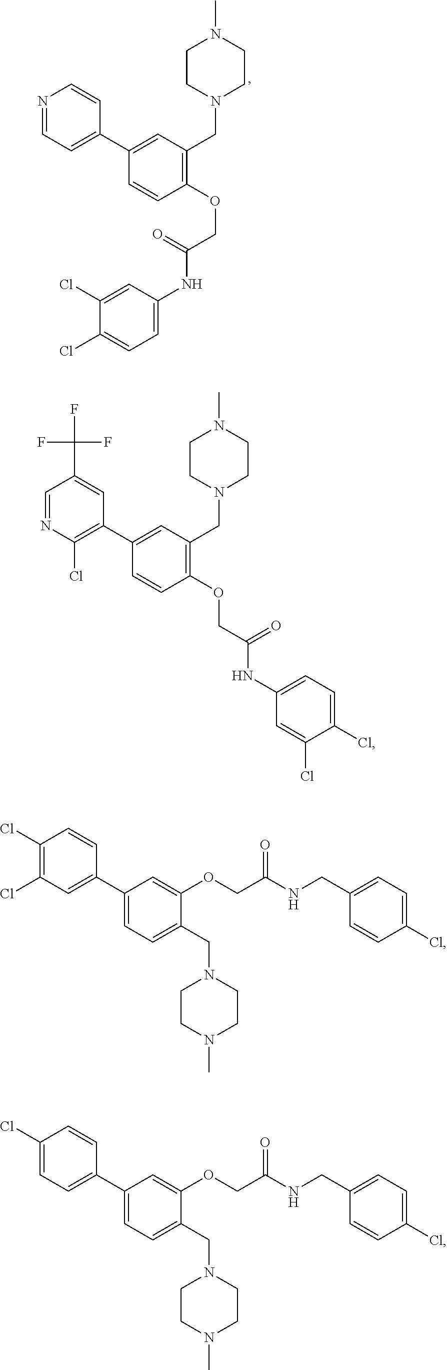

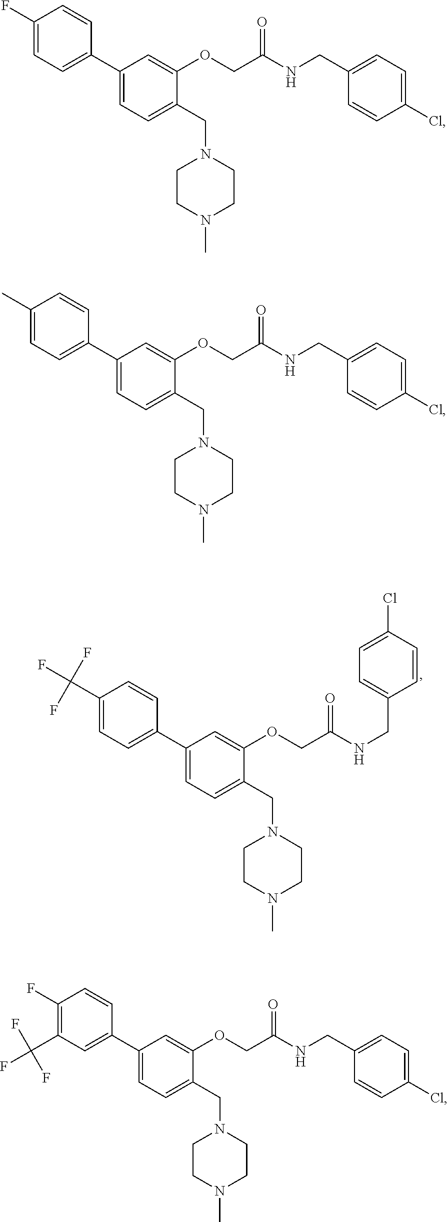

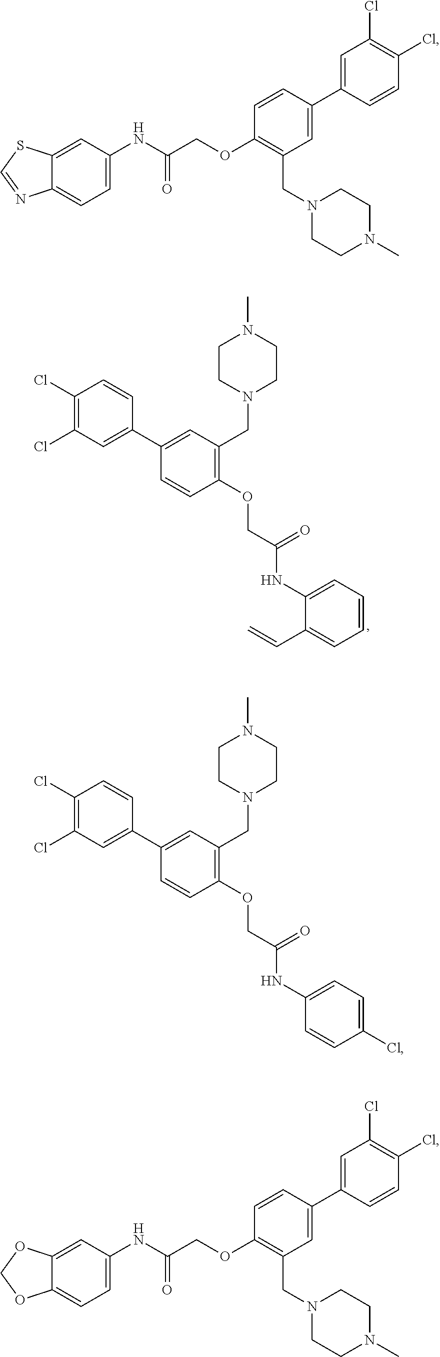

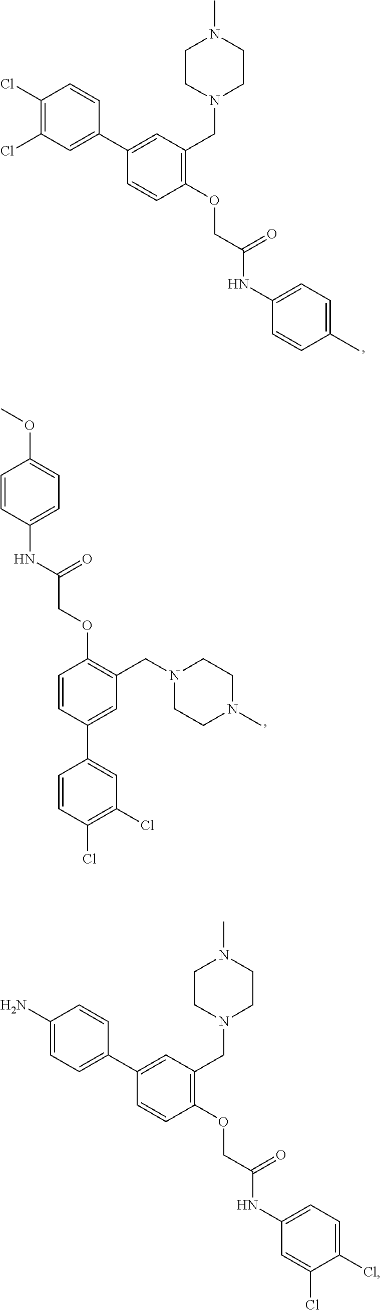

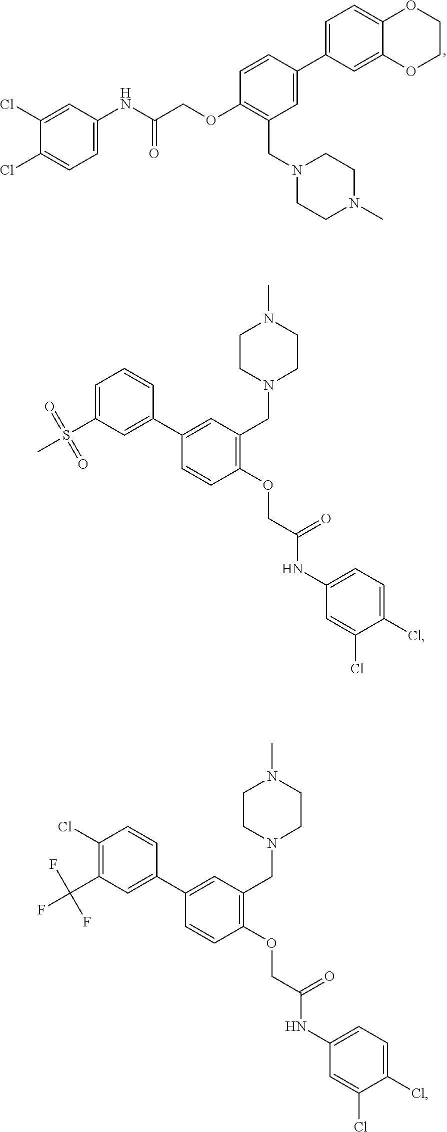

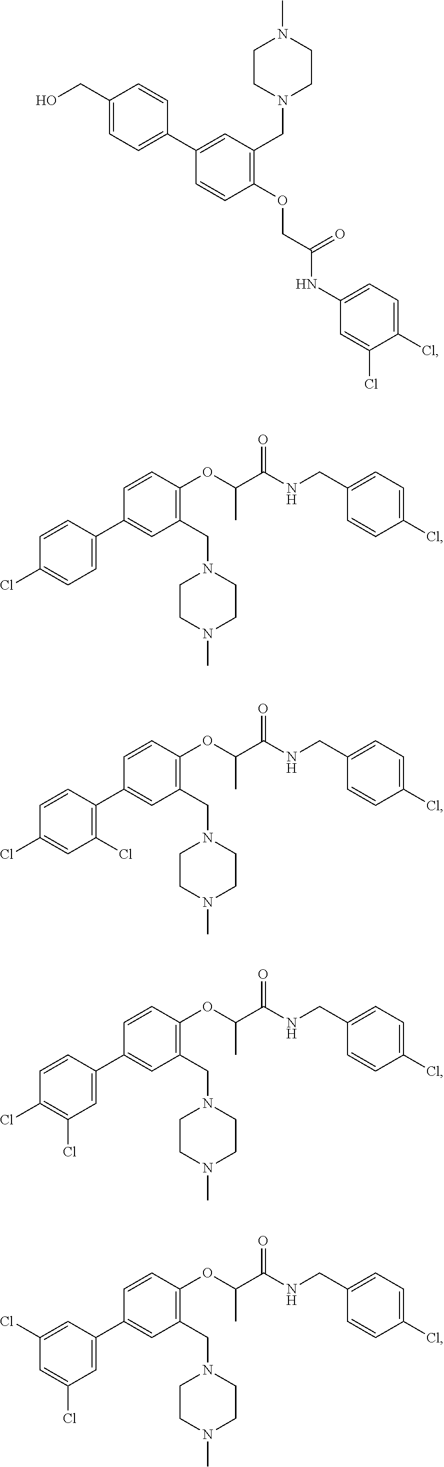

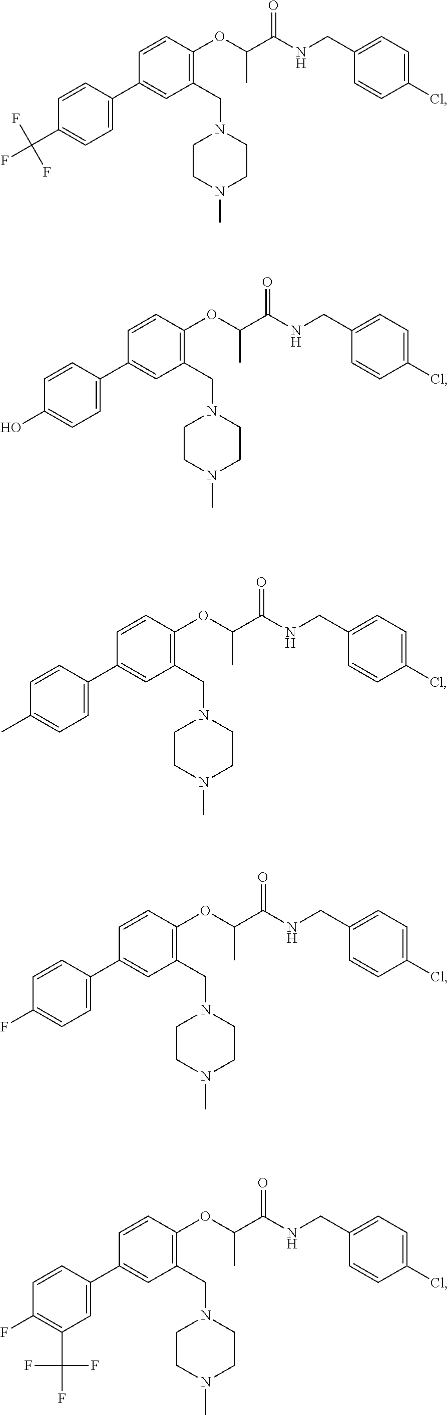

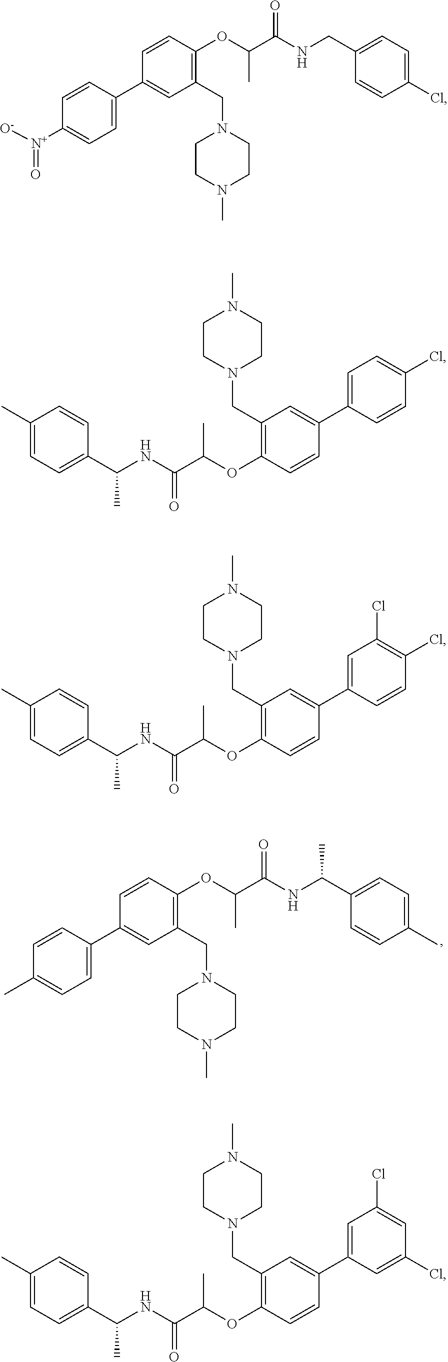

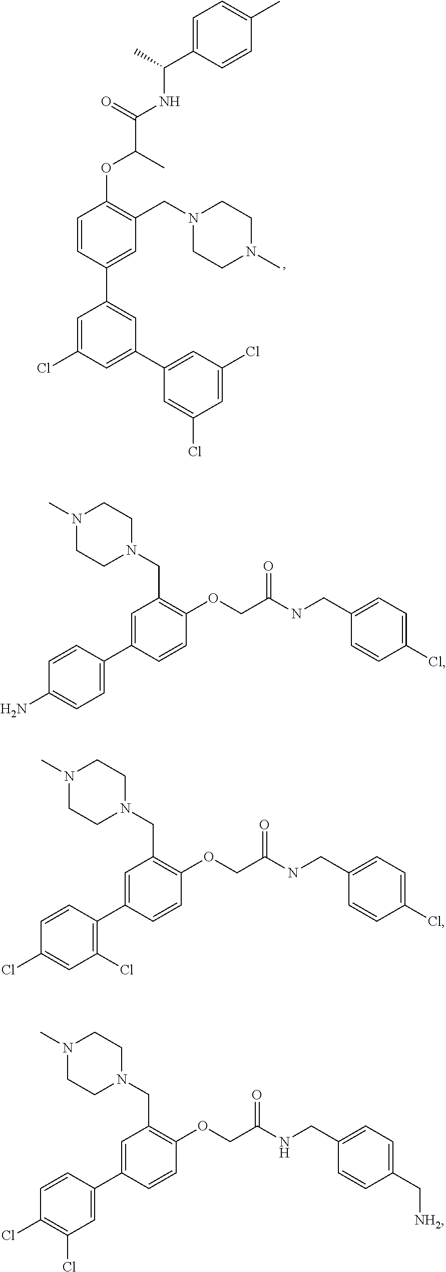

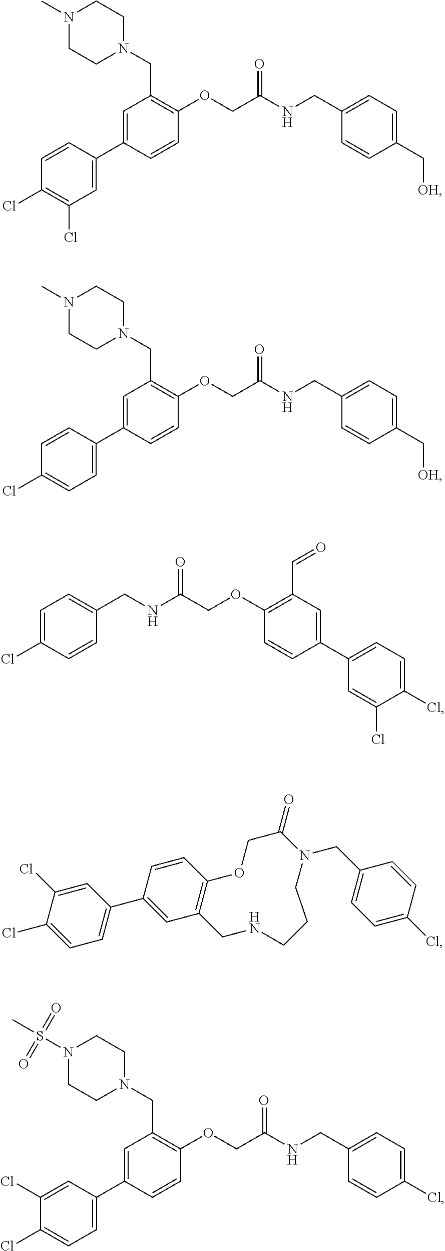 [SMILES: CN1CCN(Cc2cc(-c3ccc(Cl)c(Cl)c3)ccc2OCC(=O)NCc2ccc(CO)cc2)CC1 ; CN1CCN(Cc2cc(-c3ccc(Cl)cc3)ccc2OCC(=O)NCc2ccc(CO)cc2)CC1 ; O=Cc1cc(-c2ccc(Cl)c(Cl)c2)ccc1OCC(=O)NCc1ccc(Cl)cc1 ; O=C1COc2ccc(-c3ccc(Cl)c(Cl)c3)cc2CNCCCN1Cc1ccc(Cl)cc1 ; CS(=O)(=O)N1CCN(Cc2cc(-c3ccc(Cl)c(Cl)c3)ccc2OCC(=O)NCc2ccc(Cl)cc2)CC1]

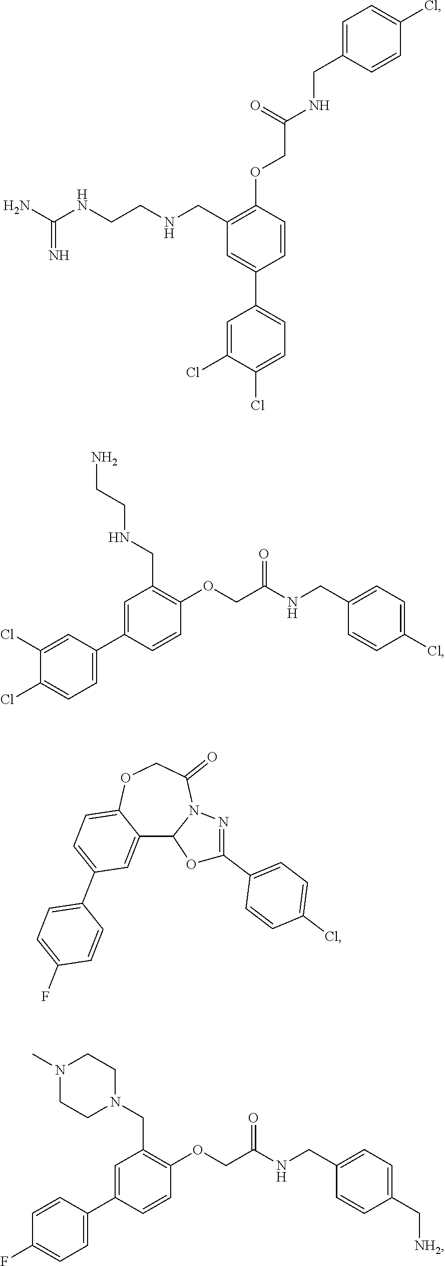 [SMILES: N=C(N)NCCNCc1cc(-c2ccc(Cl)c(Cl)c2)ccc1OCC(=O)NCc1ccc(Cl)cc1 ; NCCNCc1cc(-c2ccc(Cl)c(Cl)c2)ccc1OCC(=O)NCc1ccc(Cl)cc1 ; O=C1COc2ccc(-c3ccc(F)cc3)cc2C2OC(c3ccc(Cl)cc3)=NN12 ; CN1CCN(Cc2cc(-c3ccc(F)cc3)ccc2OCC(=O)NCc2ccc(CN)cc2)CC1]

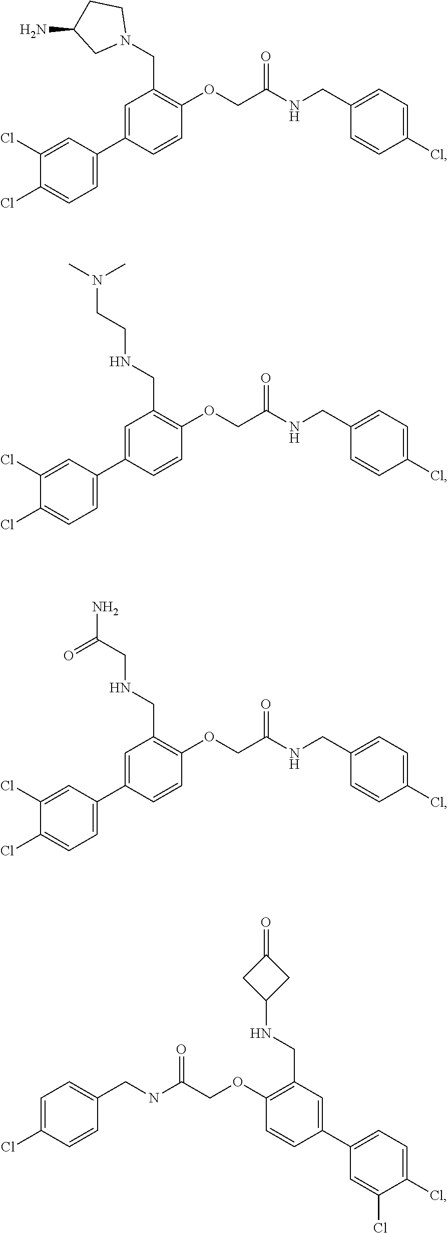

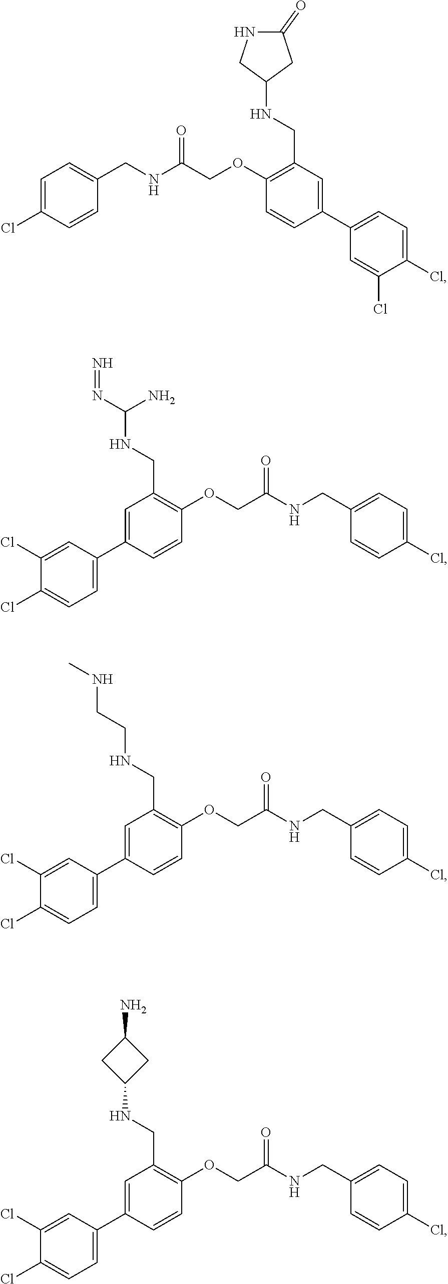

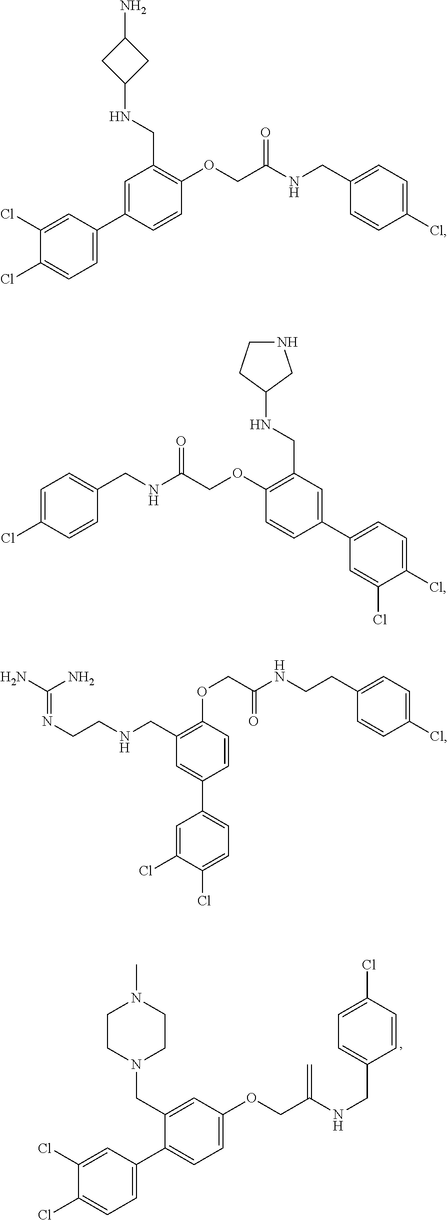 [SMILES: NC1CC(NCc2cc(-c3ccc(Cl)c(Cl)c3)ccc2OCC(=O)NCc2ccc(Cl)cc2)C1 ; O=C(COc1ccc(-c2ccc(Cl)c(Cl)c2)cc1CNC1CCNC1)NCc1ccc(Cl)cc1 ; NC(N)=NCCNCc1cc(-c2ccc(Cl)c(Cl)c2)ccc1OCC(=O)NCCc1ccc(Cl)cc1 ; C=C(COc1ccc(-c2ccc(Cl)c(Cl)c2)c(CN2CCN(C)CC2)c1)NCc1ccc(Cl)cc1]

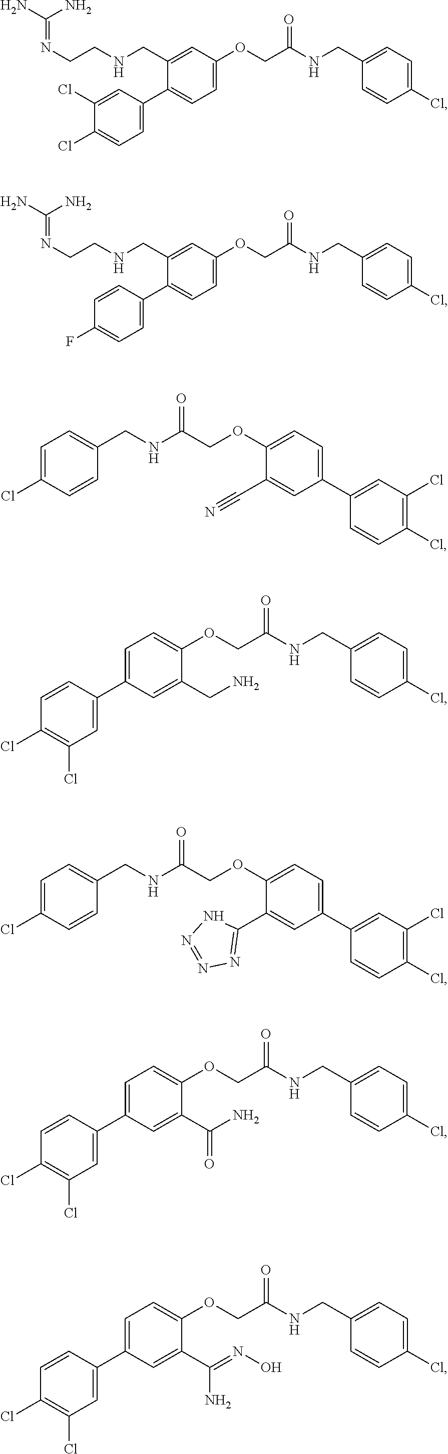

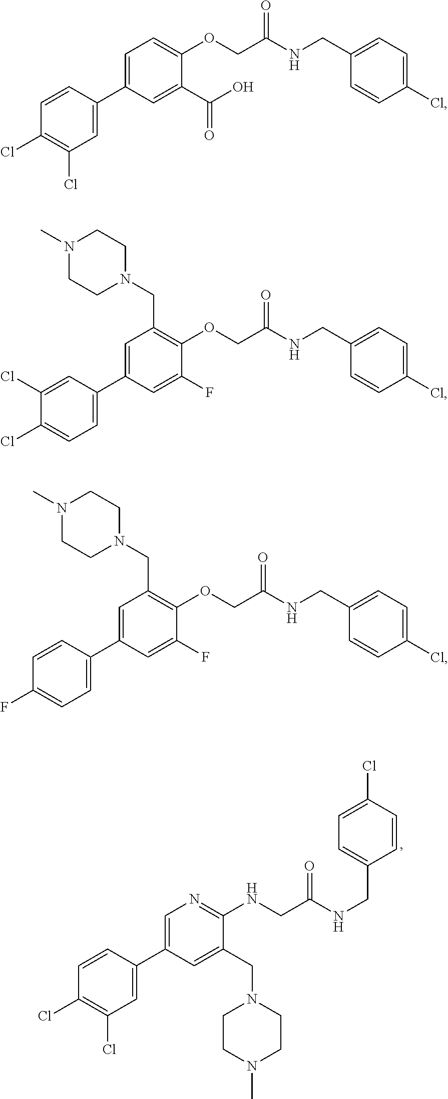 [SMILES: O=C(COc1ccc(-c2ccc(Cl)c(Cl)c2)cc1C(=O)O)NCc1ccc(Cl)cc1 ; CN1CCN(Cc2cc(-c3ccc(Cl)c(Cl)c3)cc(F)c2OCC(=O)NCc2ccc(Cl)cc2)CC1 ; CN1CCN(Cc2cc(-c3ccc(F)cc3)cc(F)c2OCC(=O)NCc2ccc(Cl)cc2)CC1 ; CN1CCN(Cc2cc(-c3ccc(Cl)c(Cl)c3)cnc2NCC(=O)NCc2ccc(Cl)cc2)CC1]

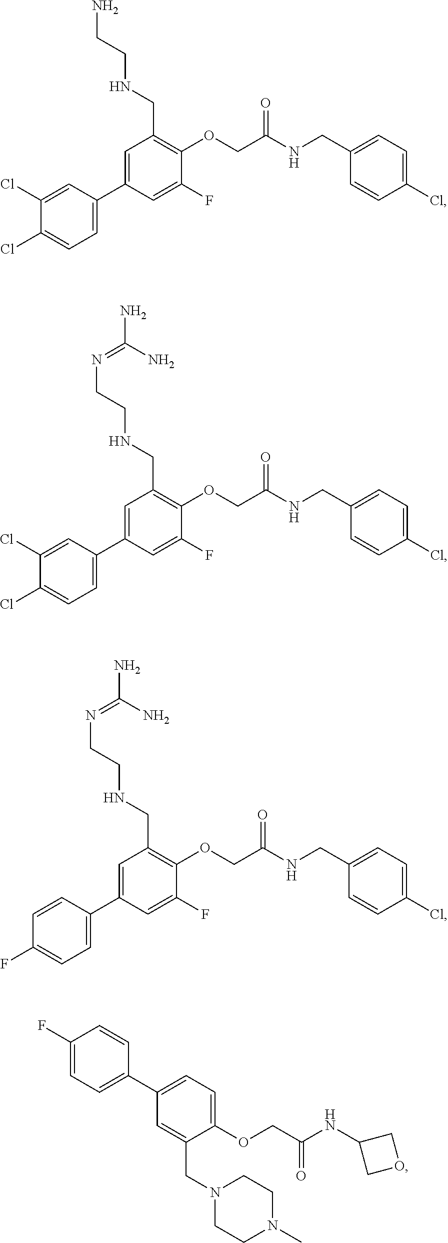 [SMILES: NCCNCc1cc(-c2ccc(Cl)c(Cl)c2)cc(F)c1OCC(=O)NCc1ccc(Cl)cc1 ; NC(N)=NCCNCc1cc(-c2ccc(Cl)c(Cl)c2)cc(F)c1OCC(=O)NCc1ccc(Cl)cc1 ; NC(N)=NCCNCc1cc(-c2ccc(F)cc2)cc(F)c1OCC(=O)NCc1ccc(Cl)cc1 ; CN1CCN(Cc2cc(-c3ccc(F)cc3)ccc2OCC(=O)NC2COC2)CC1]

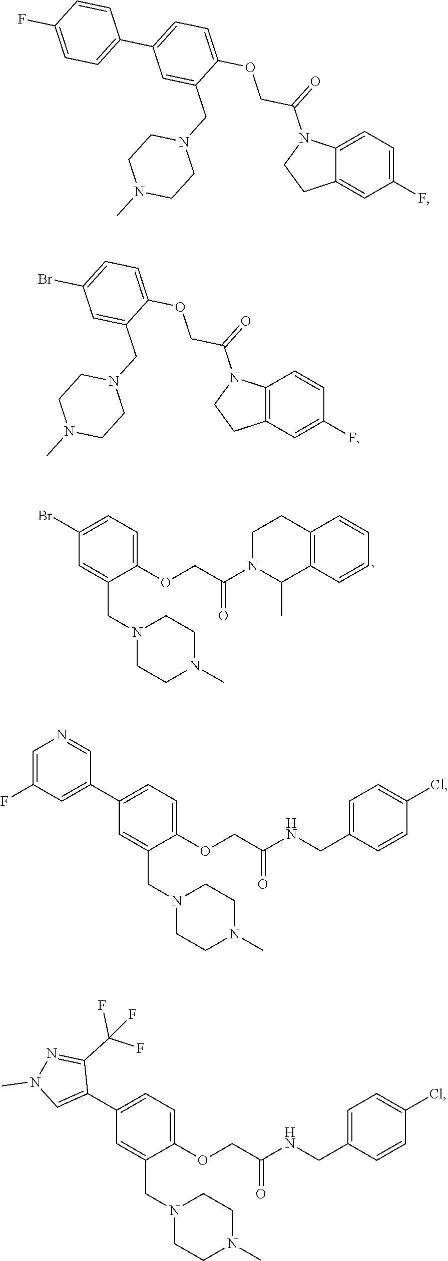 [SMILES: CN1CCN(Cc2cc(-c3ccc(F)cc3)ccc2OCC(=O)N2CCc3cc(F)ccc32)CC1 ; CN1CCN(Cc2cc(Br)ccc2OCC(=O)N2CCc3cc(F)ccc32)CC1 ; CC1c2ccccc2CCN1C(=O)COc1ccc(Br)cc1CN1CCN(C)CC1 ; CN1CCN(Cc2cc(-c3cncc(F)c3)ccc2OCC(=O)NCc2ccc(Cl)cc2)CC1 ; CN1CCN(Cc2cc(-c3cn(C)nc3C(F)(F)F)ccc2OCC(=O)NCc2ccc(Cl)cc2)CC1]

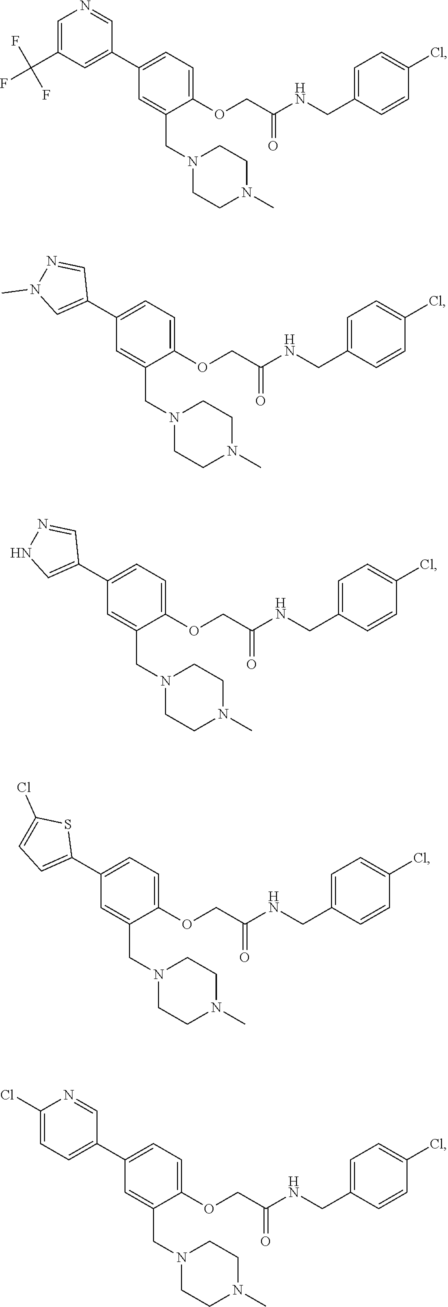 [SMILES: CN1CCN(Cc2cc(-c3cncc(C(F)(F)F)c3)ccc2OCC(=O)NCc2ccc(Cl)cc2)CC1 ; CN1CCN(Cc2cc(-c3cnn(C)c3)ccc2OCC(=O)NCc2ccc(Cl)cc2)CC1 ; CN1CCN(Cc2cc(-c3cn[nH]c3)ccc2OCC(=O)NCc2ccc(Cl)cc2)CC1 ; CN1CCN(Cc2cc(-c3ccc(Cl)s3)ccc2OCC(=O)NCc2ccc(Cl)cc2)CC1 ; CN1CCN(Cc2cc(-c3ccc(Cl)nc3)ccc2OCC(=O)NCc2ccc(Cl)cc2)CC1]

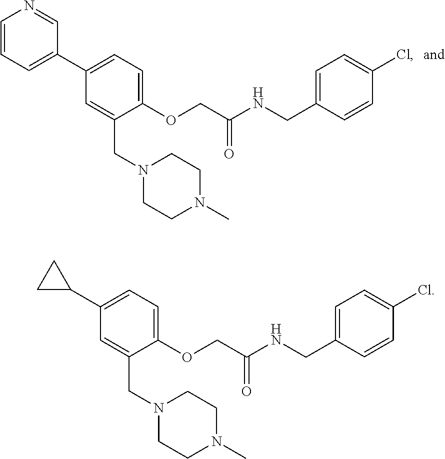 [SMILES: CN1CCN(Cc2cc(-c3cccnc3)ccc2OCC(=O)NCc2ccc(Cl)cc2)CC1 ; "and" ; CN1CCN(Cc2cc(C3CC3)ccc2OCC(=O)NCc2ccc(Cl)cc2)CC1]

16. A pharmaceutical composition comprising the compound according to claim 1, and a suitable pharmaceutical carrier, diluent, or excipient.

17. A method of treating a disease or disorder associated with TRIP8b activity in a subject in need thereof, the method comprising administering an effective amount of the compound of claim 1 for inhibiting TRIP8b activity in the subject.

18. The method of claim 17, wherein the disease or disorder is a neurological disease or disorder.

19. The method of claim 17, wherein the disease or disorder is depression.

20. The method of claim 17, wherein the disease or disorder is major depressive disorder.

21. A method for inhibiting the interaction between TRIP8b and one or more subunits of HCN, the method comprising contacting a cell expressing TRIP8b and the one or more subunits of HCN.
